(19) United States
(12) Patent Application Publication
(10) Pub. No.: US 2026/0028572 A1
(43) Pub. Date: Jan. 29, 2026

(54) BIOREACTOR FOR CELLULAR THERAPEUTICS

(71) Applicant: BRUKER CELLULAR ANALYSIS, INC., Emeryville, CA (US)

(72) Inventors: Jonathan Cloud Dragon Hubbard, Oakland, CA (US); Vivek Jadhav, Union City, CA (US); Darcy K. Kelly-Greene, Emeryville, CA (US); Kelek Olais, Emeryville, CA (US); Angel Navas Angeles, Emeryville, CA (US); Chase McRobie, Emeryville, CA (US)

(73) Assignee: BRUKER CELLULAR ANALYSIS, INC., Emeryville, CA (US)

(21) Appl. No.: 18/993,399

(22) PCT Filed: Jul. 11, 2023
(86) PCT No.: PCT/US2023/069944

§ 371 Date: Jan. 10, 2025

Related U.S. Application Data
(60) Provisional application No. 63/388,581, filed on Jul. 12, 2022.
(60) Provisional application No. 63/485,865, filed on Feb. 17, 2023.

(51) Int. Cl. C12M 1/26; C12M 1/00

(52) U.S. Cl. CPC C12M 33/14; C12M 29/00; C12M 29/04; C12M 29/10

(57) ABSTRACT

A bioreactor for expanding a population of biological cells comprises a housing having an interior cavity and a first membrane affixed within the housing. The first membrane divides the interior cavity into a chamber and a first flow channel. The bioreactor further comprises a cell culture surface contained within the chamber on which the population of biological cells may be disposed, a first inlet port fluidly coupled to the first flow channel, such that first media can be flowed into the first flow channel, and a first outlet port fluidly coupled to the first flow channel, such that the first media can be flowed out of the first flow channel. The first membrane is substantially permeable to one or more of nutrient, reagent, and gas contained within the first media and waste produced by the population of biological cells, while being substantially impermeable to the population of biological cells.

### FIELD

The present disclosure relates generally to bioreactors, and more particularly, to bioreactors for expanding cell populations.

### BACKGROUND

Cellular therapeutics offer a potentially powerful approach to successfully treating many different diseases. To date, though, few cellular therapies have been approved for use in patients, in part due to the difficulties of manufacturing the therapies in a consistent and predictable manner. Moreover, the available approaches for cell therapy manufacturing have been cost-prohibitive and lack scalability. Product quality and release testing are a significant portion of both the cost and lead time for the manufacture and delivery of a cell therapy to a patient.

Adoptive immunotherapy with ex vivo-derived T lymphocytes (T-cells) is a rapidly progressing field that has application for the treatment of disease in a range of clinical settings, including viral diseases associated with immune-compromise and cancer. T lymphocytes (T-cells) play a specialized, critical role in the antigen-specific immune response. A single administration of adoptive T-cell therapy can require billions of human T-cells, and thus, optimized protocols for the scalable manufacture of human T lymphocytes (T-cells) are essential to maximize the therapeutic potential of patient-derived T-cells. Obtaining billions of T-cells for cell therapy requires a large-scale production of T-cells that involves ex vivo culturing and expansion of a population of donor T-cells many fold in a highly controlled environment to produce a sufficient number of T-cells. Typically, T-cells may be expanded in cell culture chamber (e.g., a bioreactor) in which a culture media may be introduced and removed to provide nutrient, reagents, pH modulation, gas diffusion, temperature control, removal of wastes, etc., thereby providing an optimal environment that promote growth and/or expansion of T-cell populations. It is important that such culture media be introduced into and removed from the bioreactor without disturbing the T-cell population.

Various type of bioreactors for culturing and expanding T-cells exist on the market. For example, static bioreactors, such as a cell culture flask, need to be mixed and require a lot of surface area exposed to air to get appropriate gas diffusion to the T-cells. Some static bioreactors are gas permeable, but have limited scalability and process control due to difficulty in rapidly replacing the perfusion media. Furthermore, such static bioreactors often require high media volumes, such that the seeding cell density is higher. Hollow-fiber bioreactors are largely designed for extracting soluble proteins and perfusion media from a suspension of T-cells, which applies a lot of shear stress to the T-cells, thereby hindering the growth and/or expansion of the T-cells.

There, thus, is a need for an improve bioreactor for expanding a population of T-cells that is scalable and allows for the introduction and removal of culture media without disturbing the T-cell population.

### SUMMARY

In accordance with one aspect of the present inventions, a bioreactor for expanding a population of biological cells is provided.

The bioreactor comprises a housing having an interior cavity and a first membrane affixed within the housing. The first membrane divides the interior cavity into a chamber and a first flow channel. The first membrane is substantially permeable to one or more of nutrient, reagent, and gas contained within the first media and waste produced by the population of biological cells, while being substantially impermeable to the population of biological cells. The first membrane may have a thickness, e.g., in the range of 10-100 microns. The bioreactor further comprises a cell culture surface contained within the chamber on which the population of biological cells may be disposed. The cell culture surface may be, e.g., formed on the first membrane or may be spaced apart (e.g., at least one 1 mm) from the first membrane, e.g., on a wall of housing opposite the first membrane. The bioreactor further comprises a first inlet port fluidly coupled to the first flow channel, such that first media can be flowed into the first flow channel, and a first outlet port fluidly coupled to the first flow channel, such that the first media can be flowed out of the first flow channel.

In one embodiment, the chamber is a static chamber. In another embodiment, the chamber is a second flow channel, in which case, the bioreactor may further comprise a second inlet port fluidly coupled to the second flow channel, such that second media containing biological cells can be flowed into the second flow channel, and a second outlet port fluidly coupled to the second flow channel, such that third media containing an expanded population of biological cells may be flowed out of the second flow channel. In this embodiment, each of the first flow channel and the second flow channel may be planar and/or patterned (e.g., each of the first flow channel and the second flow channel may have a serpentine pattern), and may have a volume in the range of 2-100 ml. In this embodiment, the first membrane may be a porous membrane configured for allowing perfusion of the second media from the second flow channel into the first flow channel, while filtering the population of biological cells from the second media, such that the population of biological cells are retained within the second flow channel. Such porous membrane may be composed of, e.g., a polycarbonate. The porous membrane may have diameters in the range of 0.05-0.4 microns, and may have a porosity in the range of 1%-20%. In this embodiment, the bioreactor may further comprise at least one permeable support structure (e.g., a woven mesh) configured for reducing a lateral flex of the porous membrane in response to a pressure differential between the first flow channel and the second flow channel. Each of the permeable support structure(s) may have a porosity, e.g., in the range of 10-90%.

In another embodiment, the bioreactor comprises a second membrane affixed within the housing, with the chamber being disposed between the first membrane and the second membrane, and the second membrane being substantially permeable to gas, while being substantially impermeable to the population of biological cells and the second media. In this embodiment, the second membrane is configured for allowing diffusion of the gas from a space exterior to the second flow channel (e.g., an ambient environment) into the chamber, thereby facilitating expansion of the population of biological cells within the second flow channel. The second membrane may be a dense membrane, e.g., silicone, and may have a thickness, e.g., in the range of 50-250 microns. In this embodiment, the cell culture surface may be formed on the second membrane. The bioreactor may further comprise a permeable support structure (e.g., a honeycomb structure composed of one of, e.g., polycarbonate, polystryrene, cyclic olefin copolymer (COC), cyclic olefin polymer (COP), polypropylene, and stainless steel) configured for reducing a lateral flex of the second membrane in response to a pressure differential between the chamber and the space exterior to the second flow channel. The permeable support structure may have a thickness in the range of 0.1-2 mm and a porosity in the range of 10-90%.

In accordance with a second aspect of the present inventions, a bioreactor system is provided. The bioreactor system comprises the aforementioned bioreactor with the first flow channel and the second flow channel, and a pump assembly (e.g., comprising a peristaltic pump) coupled to the first inlet port and the second inlet port. In one embodiment, the bioreactor system further comprises a valve assembly configured for alternately allowing and preventing the flow of fluid through each of the first inlet port, the first outlet port, the second inlet port, and the second outlet port.

In accordance with a third aspect of the present inventions, a method of operating the aforementioned bioreactor with the chamber and the first flow channel is provided. The method comprises seeding the chamber with the population of biological cells (e.g., T lymphocytes (T-cells)), such that the population of biological cells are disposed on the cell culture surface. The method further comprises flowing the first media through the first inlet port and into the first flow channel (e.g., via pumping), conveying one or more of the nutrient, reagent, and gas from the first flow channel, through the first membrane, and into the chamber (e.g., via diffusion) and/or conveying the waste from the chamber, through the first membrane, and into the first flow channel (e.g., via diffusion), thereby facilitating the expansion of the population of biological cells within the chamber. The method further comprises flowing the first media out of the first flow channel through the first outlet port (e.g., via pumping) and harvesting the expanded population of biological cells from the chamber.

In accordance with a fourth aspect of the present inventions, a method of operating the aforementioned bioreactor with the first flow channel and the second flow channel is provided. The method comprises flowing the second media containing the population of biological cells (e.g., T lymphocytes (T-cells)) through the second inlet port and into the second flow channel (e.g., via pumping), such that the population of biological cells are disposed on the cell culture surface. The method further comprises flowing the first media through the first inlet port and into the first flow channel, conveying one or more of the nutrient, reagent, and gas from the first flow channel, through the first membrane, and into the second flow channel (e.g., via diffusion) and/or conveying the waste from the second flow channel, through the first membrane, and into the first flow channel (e.g., via diffusion), thereby facilitating the expansion of the population of biological cells within the second flow channel, and flowing the first media out of the first flow channel through the first outlet port (e.g., via pumping). The method further comprises flowing third media through the second inlet port and into the second flow channel, such that the expanded population of biological cells is suspended in the third media (e.g., without mechanically agitating the second flow channel), and flowing the third media with the suspended expanded population of biological cells out of the second flow channel and through the second outlet port (e.g., via pumping). An optional method further comprises perfusing the second media from the second flow channel, through the first membrane, and into the first flow channel, while substantially retaining the population of biological cells within the second flow channel, and flowing the perfused second media out of the first flow channel through the first outlet port. Another optional method further comprises flowing a wash buffer through the second inlet port and into the second flow channel, thereby washing the population of biological cells, perfusing the wash buffer from the second flow channel, through the first membrane, and into the first flow channel, while substantially retaining the population of biological cells within the second flow channel, and flowing the perfused wash buffer out of the first flow channel through the first outlet port.

In accordance with a fifth aspect of the present inventions, a method of operating the aforementioned bioreactor with the first membrane and the second membrane is provided. The method comprises seeding the chamber with the population of biological cells, such that the population of biological cells (e.g., T lymphocytes (T-cells)) are disposed on the cell culture surface. The method further comprises flowing the first media through the first inlet port and into the first flow channel (e.g., via pumping), conveying one or more of nutrient and reagent contained within the first media from the first flow channel, through the first membrane, and into the chamber (e.g., via diffusion) and/or conveying the waste from the second flow channel, through the first membrane, and into the first flow channel (e.g., via diffusion), and conveying the gas from a space exterior of the chamber, through the second membrane, and into the chamber (e.g., via diffusion), thereby facilitating the expansion of the population of biological cells within the chamber. The method further comprises flowing the first media out of the first flow channel through the first outlet port, and harvesting the expanded population of biological cells from the chamber.

In accordance with a sixth aspect of the present inventions, a method of expanding a population of biological cells (e.g., T lymphocytes (T-cells)) is provided. The method comprises seeding a chamber with the population of biological cells, such that the population of biological cells rest on a cell culture surface within the chamber. The method further comprises flowing first media containing one or more of nutrient, reagent, and gas through a first flow channel (e.g., via pumping continuously or intermittently), and diffusing the nutrient, reagent, and/or gas from the first flow channel into the chamber and/or diffusing waste produced by the population of biological cells from the chamber into the first flow channel, thereby expanding the population of biological cells within the chamber. The method further comprises harvesting the expanded population of biological cells from the chamber.

In one method, the chamber is a second flow channel, in which case, the second flow channel is seeded by flowing second media containing the biological cells into the second flow channel, such that the population of biological cells are disposed on the cell culture surface, and the expanded population of biological cells is harvested from the incubation flow channel by flowing third media into the second flow channel, such that the expanded population of biological cells is suspended in the third media (e.g., without mechanically agitating the second flow channel), and flowing the third media with the suspended expanded population of biological cells out of the second flow channel. This method may optionally comprise perfusing the second media from the second flow channel into the first flow channel, while substantially retaining the population of biological cells within the second flow channel, and flowing the perfused second media out of the first flow channel. This method may optionally comprise flowing a wash buffer within the second flow channel, thereby washing the population of biological cells, perfusing the wash buffer from the second flow channel into the first flow channel, while substantially retaining the population of biological cells within the second flow channel, and flowing the perfused wash buffer out of the first flow channel.

In accordance with a seventh aspect of the present inventions, a bioreactor for expanding a population of biological cells is provided. The bioreactor comprises a housing having an interior cavity and a porous membrane affixed within the housing. The porous membrane may have diameters in the range of 0.05-0.4 microns, may have a porosity in the range of 1%-20%, and may have a thickness in the range of 10-100 microns. The porous membrane may be composed of, e.g., a polycarbonate. The porous membrane divides the interior cavity into a first flow channel and a second flow channel. In one embodiment, each of the first flow channel and the second flow channel may be planar and/or patterned (e.g., each of the first flow channel and the second flow channel may have a serpentine pattern), and may have a volume in the range of 2-100 ml. In another embodiment, the bioreactor may further comprise at least one permeable support structure (e.g., a woven mesh) configured for reducing a lateral flex of the porous membrane in response to a pressure differential between the first flow channel and the second flow channel. Each of the permeable support structure(s) may have a porosity, e.g., in the range of 10-90%.

The bioreactor further comprises a cell culture surface contained within the second flow channel on which the population of biological cells may be disposed. The cell culture surface may be, e.g., formed on the first membrane or may be spaced apart (e.g., at least one 1 mm) from the first membrane, e.g., on a wall of housing opposite the first membrane. The bioreactor further comprises a first inlet port fluidly coupled to the second flow channel, such that first media containing biological cells can be flowed into the second flow channel. The porous membrane is configured for allowing perfusion of the first media from the second flow channel into the first flow channel, while filtering the population of biological cells from the first media, such that the population of biological cells are retained within the second flow channel. The bioreactor further comprises a first outlet port fluidly coupled to the first flow channel, such that the first media can be flowed out of the first flow channel.

In one embodiment, the porous membrane is substantially permeable to one or more of nutrient, reagent, and gas contained within second media disposed within the first flow channel and/or substantially permeable to the waste produced by the waste produced by the population of biological cells. For example, the porous membrane may be configured for allowing diffusion of the nutrient, reagent, and gas from the first flow channel into the second flow channel, and diffusion of the waste from the second flow channel into the first flow channel, thereby facilitating expansion of the population of biological cells within the chamber. In another embodiment, the bioreactor further comprises a second inlet port fluidly coupled to the first flow channel, such that the second media can be flowed into the first flow channel, and flowed out of the first outlet port. In yet another embodiment, the bioreactor further comprises a second outlet port fluidly coupled to the second flow channel, such that second media containing an expanded population of biological cells may be flowed out of the second flow channel.

In another embodiment, the bioreactor comprises a dense membrane affixed within the housing, with the second flow channel being disposed between the porous membrane and the dense membrane, and the dense membrane being substantially permeable to gas, while being substantially impermeable to the population of biological cells and liquid media. For example, the dense membrane may be configured for allowing diffusion of the gas from a space exterior to the second flow channel (e.g., an ambient environment) into the second flow channel, thereby facilitating expansion of the population of biological cells within the second flow channel. The dense membrane may be composed of, e.g., silicone, and may have a thickness, e.g., in the range of 50-250 microns. In this embodiment, the cell culture surface may be formed on the dense membrane. In this embodiment, the bioreactor may further comprise a permeable support structure (e.g., a honeycomb structure composed of one of, e.g., polycarbonate, polystryrene, cyclic olefin copolymer (COC), cyclic olefin polymer (COP), polypropylene, and stainless steel) configured for reducing a lateral flex of the second membrane in response to a pressure differential between the chamber and the space exterior to the second flow channel. The permeable support structure may have a thickness in the range of 0.1-2 mm and a porosity in the range of 10-90%.

In accordance with an eighth aspect of the present inventions, a bioreactor system is provided. The bioreactor system comprises the aforementioned bioreactor having the porous membrane and a pump assembly (e.g., comprising a peristaltic pump) fluidly coupled to the first inlet port.

In accordance with a ninth aspect of the present inventions, a method of operating the aforementioned bioreactor having the porous membrane, the first inlet port, and the second outlet port is provided. The method comprises flowing the first media containing the population of biological cells (e.g., T lymphocytes (T-cells)) through the first inlet port and into the second flow channel (e.g., via pumping), perfusing the first media from the second flow channel, through the porous membrane, and into the first flow channel, while substantially retaining the population of biological cells within the second flow channel, such that the population of biological cells are disposed on the cell culture surface, and flowing the perfused first media out of the first flow channel through the first outlet port. One method further comprises flowing a wash buffer through the first inlet port and into the second flow channel, thereby washing the population of biological cells, perfusing the wash buffer from the second flow channel, through the porous membrane, and into the first flow channel, while substantially retaining the population of biological cells within the second flow channel, and flowing the perfused wash buffer out of the first flow channel through the first outlet port.

In accordance with a tenth aspect of the present inventions, a method of operating the aforementioned bioreactor having the porous membrane, the first inlet port, the first outlet port, and the second inlet port is provided. The method comprises flowing the first media containing the population of biological cells (e.g., T lymphocytes (T-cells)) through the first inlet port and into the second flow channel (e.g., via pumping), perfusing the first media from the second flow channel, through the porous membrane, while substantially retaining the population of biological cells within the second flow channel, such that the population of biological cells are disposed on the cell culture surface, flowing the perfused first media out of the first flow channel through the first outlet port (e.g., via pumping). The method further comprises flowing the second media through the second inlet port and into the first flow channel (e.g., via pumping), conveying the one or more of nutrient, reagent, and gas from the first flow channel, through the first membrane, and into the second flow channel (e.g., via diffusion) and/or conveying waste produced by the population of biological cells from the second flow channel, through the porous membrane, and into the first flow channel (e.g., via diffusion), thereby facilitating the expansion of the population of biological cells within the second flow channel, and flowing the second media out of the first flow channel through the first outlet port (e.g., via pumping). One method further comprises flowing a wash buffer through the first inlet port and into the second flow channel, thereby washing the population of biological cells, perfusing the wash buffer from the second flow channel, through the porous membrane, and into the first flow channel, while substantially retaining the population of biological cells within the second flow channel, and flowing the perfused wash buffer out of the first flow channel through the first outlet port. Another method further comprises harvesting the expanded population of biological cells from the second flow channel.

In accordance with a tenth aspect of the present inventions, a method of operating the aforementioned bioreactor having the porous membrane, the first inlet port, the first outlet port, and the second outlet port is provided. The method comprises flowing the first media containing the population of biological cells (e.g., T lymphocytes (T-cells)) through the first inlet port and into the second flow channel (e.g., via pumping), and perfusing the first media from the second flow channel, through the porous membrane, and into the first flow channel, while substantially retaining the population of biological cells within the second flow channel, such that the population of biological cells are disposed on the cell culture surface. The method further comprises flowing the perfused first media out of the first flow channel through the first outlet port (e.g., via pumping). The method further comprises flowing the second media through the first inlet port and into the second flow channel (e.g., via pumping), such that the expanded population of biological cells is suspended in the second media (e.g., without mechanically agitating the second flow channel), and flowing the second media with the suspended expanded population of biological cells out of the second flow channel and through the second outlet port (e.g., via pumping). One method further comprises flowing a wash buffer through the first inlet port and into the second flow channel, thereby washing the population of biological cells, perfusing the wash buffer from the second flow channel, through the porous membrane, and into the first flow channel, while substantially retaining the population of biological cells within the second flow channel, and flowing the perfused wash buffer out of the first flow channel through the first outlet port.

In accordance with an eleventh aspect of the present inventions, a method of seeding a bioreactor with biological cells (e.g., T lymphocytes (T-cells)) is provided. The method comprises flowing first media containing biological cells into a first flow channel, perfusing the first media from first flow channel into the second flow channel, while substantially retaining the population of biological cells within the second flow channel, such that the population of biological cells are disposed on a cell culture surface within the first flow channel, and flowing the perfused first media out of the second flow channel. One method further comprises flowing a wash buffer within the first flow channel, thereby washing the biological cells, perfusing the wash buffer from the first flow channel into the second flow channel, while substantially retaining the biological cells within the first flow channel, and flowing the perfused wash buffer out of the second flow channel. Another method further comprises flowing second media containing one or more of nutrient, reagent, and gas through the second flow channel (e.g., via pumping continuously or intermittently), and diffusing the one or more of nutrient, reagent, and gas from the second flow channel into the first flow channel, thereby expanding the population of biological cells within the first flow channel. This method may further comprise flowing third media into the first flow channel, such that the expanded population of biological cells is suspended in the third media (e.g., without mechanically agitating the second flow channel), and flowing the third media with the suspended expanded population of biological cells out of the first flow channel.

In accordance with a twelfth aspect of the present inventions, a bioreactor comprises a housing and a first membrane within the housing and at least partially defining (i) a first flow channel on a first side of the first porous membrane for flow of first media containing one or more of nutrient and reagent and (ii) a chamber on a second side of the first membrane for a population of biological cells to be disposed within. The bioreactor further comprises a second membrane within the housing and at least partially defining an exterior space on a first side of the second membrane for introduction of a gas to the population of biological cells. The second membrane has a second side within the chamber upon which the population of biological cells is to be disposed. In one embodiment, the first membrane is configured for allowing diffusion of the one or more of nutrient and reagent from the first flow channel into the chamber, and diffusion of waste from the chamber into the first flow channel, and the second membrane is configured for allowing diffusion of the gas from a space exterior to the second flow channel into the second flow channel, thereby facilitating expansion of the population of biological cells within the second flow channel. The first membrane may have a thickness, e.g., in the range of 10-100 microns, and the second membrane may have a thickness, e.g., in the range of 50-250 microns.

In one embodiment, the chamber is a static chamber. In another embodiment, the chamber is a second flow channel for flow of second media containing the population of biological cells. In this embodiment, each of the first flow channel and the second flow channel may be planar and/or patterned (e.g., each of the first flow channel and the second flow channel may have a serpentine pattern), and may have a volume in the range of 2-100 ml. In still another embodiment, the bioreactor may further comprise at least one permeable support structure (e.g., a woven mesh) configured for reducing a lateral flex of the porous membrane in response to a pressure differential between the first flow channel and the second flow channel. Each of the permeable support structure(s) may have a porosity, e.g., in the range of 10-90%.

In yet another embodiment, the first membrane is a porous membrane configured for allowing perfusion of the second media from the second flow channel into the first flow channel, while filtering the population of biological cells from the second media, such that the population of biological cells are retained within the second flow channel, while the second membrane is a dense membrane. The porous membrane may have diameters in the range of 0.05-0.4 microns, may have a porosity in the range of 1%-20%, and may have a thickness in the range of 10-100 microns. The porous membrane may be composed of, e.g., a polycarbonate, while the second membrane may be composed of, e.g., silicone. In yet another embodiment, the second membrane at least partially defines an ambient environment on first side of the second membrane for introduction of the gas to the population of biological cells. In yet another embodiment, the bioreactor may further comprise a permeable support structure (e.g., a honeycomb structure composed of one of, e.g., polycarbonate, polystryrene, cyclic olefin copolymer (COC), cyclic olefin polymer (COP), polypropylene, and stainless steel) configured for reducing a lateral flex of the second membrane in response to a pressure differential between the chamber and the space exterior to the second flow channel. The permeable support structure may have a thickness in the range of 0.1-2 mm and a porosity in the range of 10-90%.

In accordance with a thirteenth aspect of the present inventions, a bioreactor system is provided. The bioreactor system comprises the aforementioned bioreactor and a pumping assembly configured to provide the first media to the first flow channel. In one embodiment, the bioreactor further comprises a controller configured to operate the pumping assembly.

Other and further aspects and features of embodiments will become apparent from the ensuing detailed description in view of the accompanying figures.

### BRIEF DESCRIPTION OF THE DRAWINGS

The drawings illustrate the design and utility of preferred embodiments of the disclosed inventions, in which similar elements are referred to by common reference numerals. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention, which is defined only by the appended claims and their equivalents. In addition, an illustrated embodiment of the disclosed inventions needs not have all the aspects or advantages shown. Further, an aspect or an advantage described in conjunction with a particular embodiment of the disclosed inventions is not necessarily limited to that embodiment and can be practiced in any other embodiments even if not so illustrated.

In order to better appreciate how the above-recited and other advantages and objects of the disclosed inventions are obtained, a more particular description of the disclosed inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a block diagram of a bioreactor system constructed in accordance with one embodiment of the present inventions;

FIG. 2 is a perspective view of one embodiment of a bioreactor that can be used in the bioreactor system of FIG. 1;

FIG. 3 is a cross-sectional view of the bioreactor of FIG. 2;

FIG. 4 is a flow diagram illustrating one method of operating the bioreactor of FIGS. 2-3 to expand a population of biological cells;

FIGS. 5A-5G illustrates a sequence of stages during operation of the bioreactor of FIGS. 2-3 in accordance with the method of FIG. 4;

FIG. 6 is a cross-sectional view of another embodiment of a bioreactor that can be used in the bioreactor system of FIG. 1;

FIG. 7 is a cross-sectional view of still another embodiment of a bioreactor that can be used in the bioreactor system of FIG. 1;

FIG. 8 is a flow diagram illustrating one method of operating the bioreactor of FIG. 7 to expand a population of biological cells;

FIGS. 9A-9E illustrates a sequence of stages during operation of the bioreactor of FIG. 7 in accordance with the method of FIG. 8;

FIG. 10 is a cross-sectional view of yet another embodiment of a bioreactor that can be used in the bioreactor system of FIG. 1;

FIG. 11 is a flow diagram illustrating one method of operating the bioreactor of FIG. 10 to expand a population of biological cells;

FIGS. 12A-12G illustrates a sequence of stages during operation of the bioreactor of FIG. 10 in accordance with the method of FIG. 11;

FIG. 13 is a cross-sectional view of yet another embodiment of a bioreactor that can be used in the bioreactor system of FIG. 1;

FIG. 14 is a flow diagram illustrating one method of operating the bioreactor of FIG. 13 to expand a population of biological cells;

FIGS. 15A-15G illustrates a sequence of stages during operation of the bioreactor of FIG. 13 in accordance with the method of FIG. 14;

FIG. 16 is an exploded view of one specific implementation of the bioreactor of FIG. 13; and

FIG. 17 is a block diagram of an experimental bioreactor system used to test the ability of the bioreactor of FIG. 13 to expand a population of biological cells.

### DETAILED DESCRIPTION OF THE ILLUSTRATED EMBODIMENTS

The bioreactors described herein provide a physiologically relevant environment for expanding a population of biological cells, and in particular, immunological cells, such as T lymphocytes (e.g., endogenous T-cells (ETCs), chimeric antigen receptor (CAR) T-cells, or engineered T-cells), natural killer (NK) cells, and/or other immune cells, in a cell therapy manufacturing system (CTMS). Alternatively, or in addition to, such population of biological cells may be, e.g., hematopoietic progenitor cells or stem cells, such as embryonic stem cells (ESCs), mesenchymal stem cells (MSCs), induced pluripotent stem cells (iPSCs), or the like.

Significantly, the bioreactors described herein are designed, such that nutrient, reagent, and gas (e.g., air, oxygen, etc.) may be efficiently delivered to the population of biological cells residing in the bioreactors, and waste may be efficiently extracted from the population of biological cells, without significantly disturbing the population of biological cells. To this end, the bioreactors described herein are designed, such that culture media for providing nutrient, reagent, and gas to the population of cells and extracting wastes from the population of cells, can be flowed through the bioreactors. A permeable membrane is used in each of the bioreactors described herein to isolate the population of biological cells from the flow culture media, allowing the nutrient, reagent, gas, and waste to diffuse between the population of biological cells and the culture media without substantially disturbing the population of biological cells, as well as preventing the passage of the population of biological cells into the cull culture media flow. The bioreactors described herein may also be designed, such a population of biological cells may be efficiently seeded within the bioreactors for incubation and expansion by flowing a media into the bioreactors, and then efficiently harvested from the bioreactors when the expanded population of biological cells are ready to be harvested by flowing media out of the bioreactors. In this case, the media for seeding the bioreactors with the population of biological cells, and harvesting the expanded population of biological cells from the bioreactors, and the culture media may be separately and independently flowed through the bioreactors. The permeable membrane may be additionally porous, such that the population of biological cells may be filtered from media as the media is flowed through the bioreactors, thereby preventing loss of biological cells when seeding the bioreactors with the population of biological cells and/or subsequently washing the population of biological cells.

The bioreactors described herein can be automated or semi-automated and are scalable, thereby maximizing, or at least increasing, the yield of biological cells. The bioreactors described herein may be incorporated into cell therapy workflow that may perform functions other than biological cell expansion, e.g., sample collection, cell sorting, cell modification, quality control assaying, formulation and filling, treatment administration, etc. The bioreactors described herein may serve as stand-alone units or may be integrated (e.g., as a cartridge or cassette) within the CTMS, which can receive, controllably manipulate, and monitor the cartridge or cassette. The cartridge or cassette may have a sealed enclosure, which may be sterile and/or hermetically sealed, and one or more inlet and/or outlet ports for fluidly accessing the sealed enclosure, e.g., the bioreactor within the cartridge or cassette. In addition to a bioreactor, such cartridge or cassette may include other components, regions for cell monitoring and/or assaying, reagent reservoirs, and the like. Further details of an exemplary CTMS are set forth in PCT Application Ser. No. PCT/US2022/012194, entitled “Systems, Apparatuses, and Methods for Cellular Therapeutics Manufacture, which is expressly incorporated herein by reference.

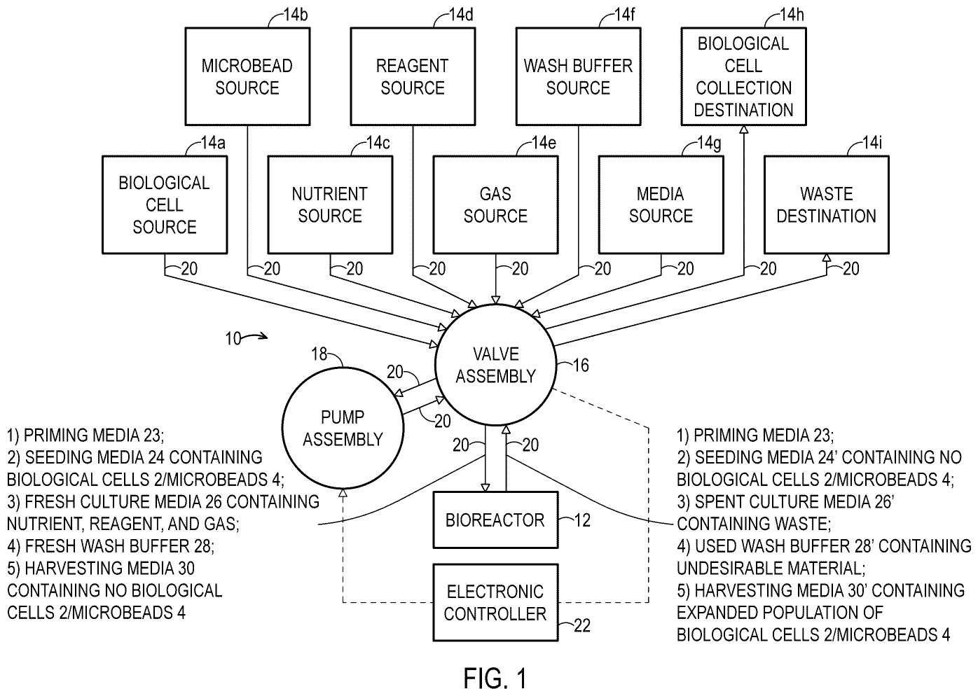

Referring first to FIG. 1, one embodiment of a bioreactor system 10 constructed in accordance with the present inventions will be described. The bioreactor system 10 generally comprises a bioreactor 12, an arrangement of sources/destinations 14, a valve assembly 16, a pump assembly 18, an arrangement of fluid conduits 20, and an electric controller 22.

The bioreactor 12 is generally configured for incubating and expanding a seed population of cells, as will be described in further detail below. The arrangement of fluid conduits 20 fluidly couple the bioreactor 12, arrangement of sources/destinations 14, valve assembly 16, and pump assembly 18 together to facilitate the flow of different media between these components. The arrangement of fluid conduits 20 may, e.g., take the form of flexible plastic tubing and/or rigid channels formed within a monolithic structure.

The arrangement of sources/destinations comprises a biological cell source 14a, a microbead source 14b, a nutrient source 14c (e.g., carbohydrates, lipids, proteins, and nucleic acids), a reagent source 14d (e.g., cytokines, such as Interleukin-2 (IL-2), or transfection reagent for converting T-cells into chimeric antigen receptor (CAR) T-cells), a gas source 14e (e.g., air), a wash buffer source 14f, a media source 14g (e.g., Phosphate Buffered Saline (PBS)), a biological cell collection destination 14h, and a waste destination 14i. Each of the sources 14a-14d and 14f-14g may, e.g., take the form of a bag supplied with the respective component (i.e., a seed population of biological cells, microbeads, nutrient, reagent, wash buffer, and media), and each of the destinations 14h-14i may take the form of a bag for subsequently collecting the respective component (i.e., an expanded population of biological cells and waste). The gas source 14e may, e.g., the take the form of an ambient environment, although in alternative embodiments, the gas source may take the form of a gas chamber. The biological cells, microbeads, nutrient, and reagent may be combined with an aqueous media to facilitate their flow within the bioreactor system 10. In alternative embodiments, any of the sources/destinations 14a-14i may take form of rigid chambers, e.g., formed within a monolithic structure. In other alternative embodiments, some of the sources can be combined or consolidated. For example, the biological cell source 14a and microbead source 14b can be combined into a single biological cell/microbead source. As another example, the biological cell source 14a, nutrient source 14c, and wash buffer source 14f may be combined into a single biological cell/nutrient/wash buffer source. As still another example, the microbead source 14b and reagent source 14d may be combined into a single microbead/reagent source. As still another example, the nutrient source 14c and gas source 14f may be combined into a single nutrient/gas source. As still another example, the biological cell source 14a, microbead source 14b, nutrient source 14c, reagent source 14d, gas source 14e, and wash buffer source 14f can be combined into a single biological cell/microbead/nutrient/reagent/gas/wash buffer source. It should also be appreciated that additional sources and/or destinations can be added to the list of sources and destinations listed above. For example, a source of cryopreservation media can be used to help preserve the biological cells after expansion.

The valve assembly 16 is configured for selectively defining the flow paths (shown by arrows) between various ones of the sources/destinations 14 and the bioreactor 12, while the pump assembly 18 is configured for providing the necessary fluid pressure for conveying various media along the flow paths defined by the valve assembly 16, such that different media can be flowed between the sources/destinations 14 and the bioreactor 12 for the purpose of priming the bioreactor 12 (i.e., removing all air within the bioreactor 12), seeding the bioreactor 12 with a population of biological cells (with or without microbeads), culturing the biological cells, such that the population of biological cells are expanded, washing the biological cells, and harvesting the expanded population of biological cells from the bioreactor 12. Although both the valve assembly 16 and pump assembly 18 are illustrated in FIG. 1 as being single components, it should be appreciated that each of the valve assembly 16 and pump assembly 18 can comprise multiple components (i.e., multiple valves or multiple pumps) that are fluidly coupled to directly to each other or indirectly to each other via other components of the bioreactor system 10 via the arrangement of fluid conduits 20.

In one embodiment, the valve assembly 16 may be configured for selectively directing (a) priming media 23 from the media source 14g to the bioreactor 12, and from the bioreactor 12 to the waste destination 14i, thereby priming the bioreactor 12; (b) seeding media 24 containing a seed population of biological cells 2 or microbeads 4, or both, from the biological cell source 14a and/or microbead source 14b to the bioreactor 12; and directing seeding media 24′ without the biological cells 2 from the bioreactor 12 to the waste destination 14i, thereby seeding the bioreactor 12 with a population of biological cells 2; (c) directing fresh culture media 26 containing nutrient, reagent, and gas from the nutrient source 14c, reagent source 14d, and gas source 14e (or alternatively directing gas separately from culture media 26 from the gas source 14e) to the bioreactor 12; and directing spent culture media 26′ containing waste from the population of biological cells 2 contained within the bioreactor 12 to the waste destination 14i, thereby culturing and facilitating expansion of the population of biological cells 2 contained within the bioreactor 12; (d) directing fresh wash buffer 28 from the wash buffer source 14f to the bioreactor 12, and directing used washer buffer 28′ with undesirable material (e.g., unbound molecules, debris, dead cells, or other unwanted molecules) from the bioreactor 12 to the waste destination 14i, thereby washing the population of biological cells 2 and microbeads 4; and (e) directing harvesting media 30 containing no biological cells 2 and microbeads 4 from the media source 14g to the bioreactor 12, and directing the harvesting media 30′ with the expanded population of biological cells 2 and microbeads 4 from the bioreactor 12 to the biological cell collection destination 14h, thereby harvesting the expanded population of biological cells from the bioreactor 12.

The electric controller 22 is configured for controlling the operation of the valve assembly 16 and pump assembly 18 in accordance with one or more programs stored within memory (not shown) to facilitate the various flow processes described herein, including priming the bioreactor 12, seeding the bioreactor 12 with the population of biological cells 2 and microbeads 4, culturing the population of biological cells 2, washing the population of biological cells 2 and microbeads 4, and once sufficiently expanded, harvesting the expanded population of biological cells 2 and microbeads 4 from the bioreactor 12. The electric controller 22 can comprise, e.g., a processor, application specific integrated circuit (ASIC), field programmable gate array (FPGA), or other type of circuitry.

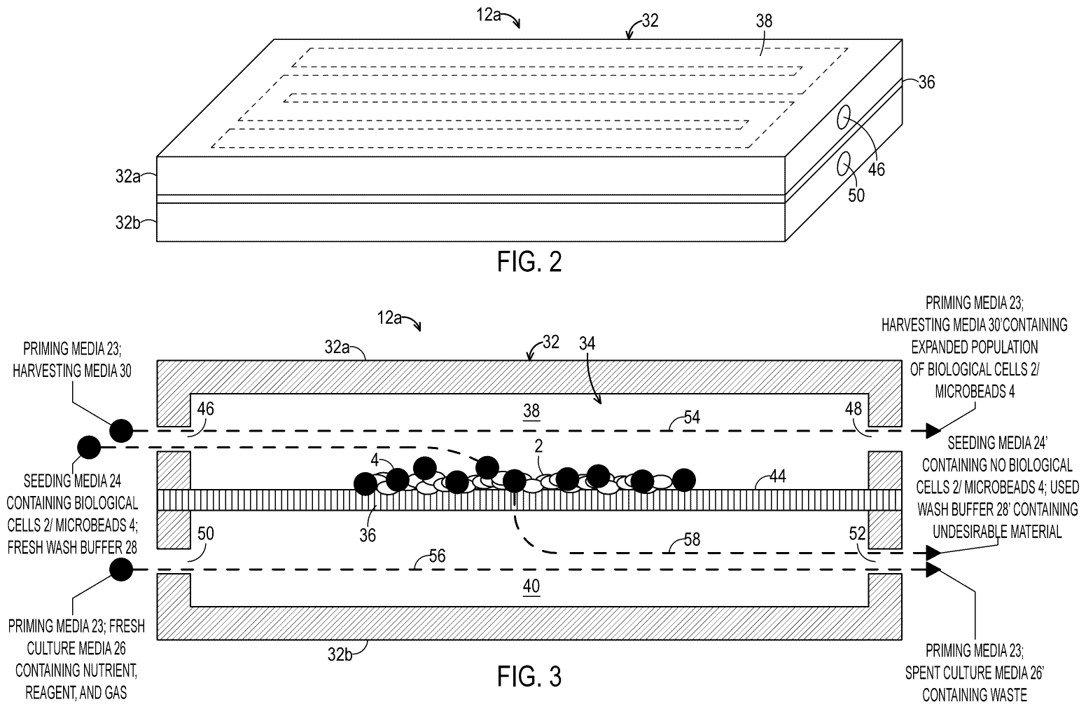

Referring now to FIGS. 2 and 3, one embodiment of a bioreactor 12a for use in the bioreactor system 10 of FIG. 1 will now described.

The bioreactor 12a generally comprises a housing 32 having an interior cavity 34. The outer periphery of the housing 32, and thus the interior cavity 34, may be planar in nature (i.e., the length (x-dimension) and width (y-dimension) are much greater than the height or thickness (z-dimension)) to maximize the gas and nutrient transfer rate within the bioreactor 12, as well as to minimize the volume of media that flows through the bioreactor 12a, as will be described in further detail below. Furthermore, the outer periphery of the housing 32, and thus the interior cavity 34, has a suitable size that promotes the expansion of a population biological cells 2 (i.e., increasing the number of biological cells 2) within the bioreactor 12, e.g., sufficiently small enough to provide the necessary density for exhibiting cross-signaling within the population of biological cells 2, but large enough to avoid overcrowding within the population of biological cells 2.

In the illustrated embodiment, the housing 32 comprises a first (upper) hollow housing portion 32a and a second (lower) hollow housing portion 32b affixed together using suitable means (e.g., ultrasonic welding, adhesive, screws, or bolts) to form the interior cavity 34 therein. The upper and lower housing portions 32a, 32b may be composed of a suitable rigid material capable of withstanding fluid pressure within the interior cavity 34. As one example, the upper and lower housing portions 32a, 32b can be composed of a biocompatible material that is easy to manufacture, transparent, non-autofluorescent, and suitable for bonding to other plastics or elastomers. For example, the upper and lower housing portions 32a, 32b may be composed of a rigid biocompatible polymer material, such as, e.g., polycarbonate, polystyrene, cyclic olefin copolymer (COC), cyclic olefin polymer (COP), etc. In the preferred embodiment, the outer periphery of the lower housing portion 32b May have the same size as the outer periphery of the upper housing portion 32a, although in alternative embodiments, the outer periphery of the lower housing portion 32b may differ from the outer periphery of the upper housing portion 32a. In one example, the dimensions of the outer periphery of each of the upper and lower housing portions 32a, 32b may be in the range of 5 cm (width)×5 cm (length)×0.1 cm (height or thickness) to 30 cm (width)×30 cm (length)×30 cm (height or thickness). In one exemplary embodiment, the dimensions of the outer periphery of each of the upper and lower housing portions 32a, 32b is 10 cm (width)×10 cm (length)×1 cm (height or thickness).

The bioreactor 12a further comprises a membrane 36 affixed within the housing 32, such that the interior cavity 34 is divided into an upper flow channel 38 and a lower flow channel 40 through which different types of media can be flowed. Significantly, as will be described in further detail below, the use of flow channels 38, 40 in the bioreactor 12a provides a convenient means for seeding the bioreactor 12a with a population of biological cells 2, culturing the population of biological cells 2 with nutrient and reagent, thereby facilitating expansion of the population of biological cells 2, suspending and washing the biological cells 2, and harvesting the expanded population of biological cells 2 from the bioreactor 12a.

In the illustrated embodiment, the membrane 36 is affixed between the upper housing portion 32a and the lower housing portion 32b, such that membrane 36 is sandwiched between the housing portions 32a, 32b when they are affixed to each other. As a result, the upper flow channel 38 is formed between the upper housing portion 32a and the top surface of the membrane 36, whereas the lower flow channel 40 is formed between the lower housing portion 32b and the bottom surface of the membrane 36. The membrane 36 may be suitably bonded between the upper and lower housing portions 32a, 32b to ensure that media does not laterally leak out of the bioreactor 12a at the interfaces between the membrane 36 and housing portions 32a, 32b. The flow channels 38, 40 may be suitably formed, as portions of the interior cavity 34, into the respective housing portions 32a, 32b in any suitable manner, e.g., injection molding or machining.

In a similar manner as the outer peripheries of the housing 32, and thus the upper and lower housing portions 32a, 32b, described above, each of the flow channels 38, 40 may be planar in nature (i.e., the length (x-dimension) and width (y-dimension) are much greater than the height or thickness (z-dimension)) to maximize the gas and nutrient transfer rate between the flow channels 38, 40, as well as to minimize the volume of media that flows within the respective flow channels 38, 40. Furthermore, in a similar manner as the outer periphery of the upper housing portion 32a described above, the upper flow channel 38 has a suitable size and volume that promotes the expansion of biological cells, e.g., sufficiently small enough to provide the necessary density for exhibiting cross-signaling within the population of biological cells 2, but large enough to avoid overcrowding within the population of biological cells 2. By extension, the lower flow channel 40 may have the same size and volume as the upper flow channel 38.

For example, the dimensions of each of the flow channels 38, 40 may be in the range of 0.5 mm to 5 mm deep and 5 mm to 10 mm wide, and may have a volume in the range of 2 mL to 100 mL. In one exemplary embodiment, the dimensions of each of the flow channels 38, 40 may be 1 mm deep and 4 mm wide, and may have a volume of 4 mL. Although, in the illustrated embodiment, the dimensions and volumes of the flow channels 38, 40 are identical, the dimensions and volumes of the flow channels 38, 40 may differ from each other. In the illustrated embodiment, each of the flow channels 38, 40 is patterned (i.e., do not extend in a single straight line), and in particular, are serpentine in nature, as best illustrated in FIG. 2 (only upper flow channel 38 shown in phantom), thereby providing a more even flow across the surface area of the flow channels 38, 40, and allowing better clearance of bubbles trapped within the flow channels 38, 40, as well as more uniform nutrient distribution and cell homogeneity. In the serpentine pattern illustrated in FIG. 2, the upper flow channel 38 has a first segment that extends in a first direction for a first distance, then a second segment that extends from the first segment in second direction (e.g., perpendicular to the first segment) for a second distance smaller than the first distance; then a third segment that extends from the second segment in a third direction opposite and generally parallel to the first segment and for a third distance; then a fourth segment that extends from the third segment in a fourth direction (e.g., perpendicular to the third segment) for a fourth distance smaller than the third distance, and so forth as depicted in FIG. 2. Although, in the illustrated embodiment, the first and third distances are the same, in alternative embodiments, the first and third distances may differ from each other. In other embodiments, the different segments of the upper flow channel 38 may each extend in different directions and at different distances than that depicted in FIG. 2 to facilitate the flow of media.

The bioreactor 12a further comprises a cell culture surface 44 on which the population of biological cells 2 may be disposed. In the illustrated embodiment, the cell culture surface 44 is formed on the top surface of the membrane 36. In this manner, gravity urges the population of biological cells 2 to rest on the cell culture surface 44. The cell culture surface 44 may be smooth, such that after expansion of the population of biological cells 2, the cell culture surface 44 does not trap, and therefore easily releases, the biological cells 2, so as to not hinder harvesting of the expanded population of biological cells 2 from the upper flow channel 38. In alternative embodiments, the cell culture surface 44 may be chemically functionalized (e.g., with T cell activating agents (e.g., for antigen-specific or non-antigen-specific activation), or with surface blocking ligands and/or biocompatible polymers), biologically functionalized (e.g., biologically derived materials, such as primary and co-activating molecules), and/or structurally functionalized (e.g., with a plurality of concave features, such as dimples or grooves), as described in PCT Application Ser. No. PCT/US2022/012194, which has been previously incorporated herein by reference.

In an optional embodiment, microbeads 4 (e.g., polystyrene beads, glass beads, amorphous solid substrates, Luminex™ beads, or the like) may be disposed in the upper flow channel 38 to place the microbeads 4 in contact with the population of biological cells 2. The microbeads 4 may be liposome-coated or may include moieties/molecules covalently or non-covalently attached, such as fluorescent labels, proteins (including receptor molecules) carbohydrates, antigens, small molecule signaling moieties, or other chemical/biological species capable of use in an assay. In some embodiments, the microbeads 4 may serve as a synthetic surface for activating the biological cells 2 and may be magnetically manipulatable for sorting the population of biological cells 2 without the need for removing the population of biological cells 2 from the upper flow channel 38 until harvest.

As will be described in further detail, seeding media 24 containing a seed population of biological cells 2 (and optionally microbeads 4) can be flowed into the upper flow channel 38 to seed the bioreactor 12a with a population of biological cells 2 (and optionally microbeads 4), and fresh culture media 26 containing nutrient, reagent, and gas can be flowed (continuously or intermittently) through the lower flow channel 40, thereby providing nutrient, reagent, and gas to, and extracting waste from, the population of biological cells 2 residing in the upper flow channel 38 via the action of diffusion through the membrane 36. As a result, expansion of the population of biological cells 2 is facilitated. Optionally, a wash buffer 28 may be flowed into the upper flow channel 38 during incubation of the population of biological cells 2 to remove undesirable material (e.g., unbound molecules, debris, dead cells, or other unwanted molecules). Further details on the use of microbeads and wash buffer are described in PCT Application Ser. No. PCT/US2022/012194, which has been expressly incorporated herein by reference.

Significantly, the membrane 36 is substantially permeable to the nutrient, reagent, and gas contained in the culture media 26 and waste from the population of biological cells 2, such that the gas, nutrient, and reagent, may diffuse from the lower flow channel 40 into the upper flow channel 38 for take-up by the population of biological cells 2, and the waste may diffuse from the upper flow channel 38 into the lower flow channel 40 and away from the population of biological cells 2, thereby facilitating the expansion of the population of biological cells 2. Preferably, the membrane 36 provides a short diffusion path for the nutrient, gas, reagents, and waste between the flow channels 38, 40. Thus, the membrane 36 preferably has a relatively small thickness, e.g., in the range of 10-100 microns. In one specific embodiment, the thickness of the membrane 36 is 10 microns. For the purposes of this specification, the terms “diffuse” or “diffusion” may be defined as a process resulting from random motion of atoms, ions, or molecules (solid, liquid, or gas) by which there is a net flow of matter from a region of higher concentration to a region of lower concentration (i.e., across a chemical gradient).

Significantly, the diffusion of nutrient, reagent, gas, and waste across the membrane 36 creates a low flow between the upper flow channel 38 and the lower flow channel 40 that does not significantly disturb the population of biological cells 2. As a result, the population of biological cells 2 may remain statically on the cell culture surface 44, and in this illustrated case, on the membrane 36. Thus, the cell culture surface 44 may not need to be structurally functionalized to facilitate retention of the population of biological cell 20, thereby reducing or minimizing the chances that biological cells 2 will be trapped on the cell culture surface 44 when the expanded population of biological cells 2 are harvested from the upper flow channel 38.

In the preferred embodiment, the membrane 36 is also substantially permeable to the seeding media 24 (and optionally, wash buffer 28 along with the undesirable material), but is substantially impermeable to the biological cells 2 (and optionally, microbeads 4), such that, when the upper flow channel 38 is under pressure, the population of biological cells 2 (and optionally, microbeads 4) are substantially retained within the upper flow channel 38 while the seeding media 24′ containing no biological cells 2 and microbeads 4 (and optionally, the used wash buffer 28′ containing undesirable material) perfuses through the membrane 36 from the upper flow channel 38 into the lower flow channel 40. In essence, the membrane 36 serves to filter the biological cells 2 (and optionally, microbeads 4) from the seeding media 24′ containing no biological cells 2 and microbeads 4 (and optionally, the used wash buffer 28′ containing undesirable material) as the seeding media 26′ containing no biological cells 2 and microbeads 4 (and optionally, the wash buffer 28 containing undesirable material) perfuses through the membrane 36 from the upper flow channel 38 into the lower flow channel 40. To facilitate perfusion of seeding media 24′ containing no biological cells 2 and microbeads 4 (and optionally, the used wash buffer 28′ containing undesirable material) through the membrane 36 between the flow channels 38, 40, the membrane 36 is preferably porous, meaning that membrane 36 has a solid matrix with defined pores that have diameters ranging from 0.002 microns to 20 microns. In one example, the porous membrane 36 may have a pore diameter in the range of 0.05-0.4 microns, and a porosity in the range of 1%-20%. In one specific embodiment, the porous membrane 36 has a pore diameter of 0.2 microns, and a porosity of 10%. For the purposes of this specification the terms “perfuse” or “perfusion” may be defined as the bulk movement of a liquid from a region of higher pressure to a region of lower pressure (i.e., across a pressure gradient). In one example, the porous membrane 36 is substantially permeable to a particular component if more than 90 percent of that component can pass through the porous membrane 36 between the flow channels 38, 40, and substantially impermeable to a particular component if less than 10 percent of that component can pass through the porous membrane 36 from the upper flow channel 38 into the lower flow channel 40. Preferably, the porous membrane 36 resists flexing in the presence of a dynamic pressure differential between the flow channels 38, 40. To this end, the porous membrane 36 may be composed of any suitable durable biocompatible material, such as, e.g., polycarbonate.

The bioreactor 12a has an arrangement of inlet and outlet ports to facilitate the flow of the priming media 23, seeding media 24, culture media 26, wash buffer 28, and harvesting media 30 through the interior cavity 34 of the bioreactor 12a. In particular, the bioreactor 12a comprises an inlet port 46 and outlet port 48 associated with the upper flow channel 38, and an inlet port 50 and outlet port 52 associated with the lower flow channel 40. Although the inlet port 46 and the inlet port 50 are illustrated as being located on one side (the left side in FIG. 3) of the bioreactor 12a, and the outlet port 48 and the outlet port 52 are illustrated as being located on the other side (the right side in FIG. 3) of the bioreactor 12a, the inlet port 46 and the inlet port 50 may be located on opposite sides of the bioreactor 12a, while the outlet port 48 and the outlet port 52 may be located on opposite sides of the bioreactor 12. Furthermore, although the inlet port 46 and the outlet port 48 are illustrated as being located on opposite sides (respectively the left and right sides in FIG. 3) of the bioreactor 12a, and the inlet port 50 and outlet port 52 are illustrated as being located on opposite sides (respectively the left and right sides in FIG. 3) of the bioreactor 12a, the inlet port 46 and the outlet port 48 may be located on same side (e.g., left side) of the bioreactor 12a, while the inlet port 50 and outlet port 52 may be located on same side (e.g., the right side) of the bioreactor 12. Notably, the ports 46-52 may be external ports to which conduits can be actively connected to and disconnected from, or may be internal to a structure, such as a cassette or cartridge, to which other flow channels and/or chambers within the structure may be permanently fluidly coupled.

The valve assembly 16 (illustrated in FIG. 1) may be operated to selectively open and close the ports 46-52, such that different flow paths may be defined within the interior cavity 34 of the bioreactor 12a.

As one example, the valve assembly 16 may be operated to open the inlet port 46 and outlet port 48 associated with the upper flow channel 38, and to open the inlet port 50 and outlet port 52 associated with the lower flow channel 40, thereby respectively defining flow paths 54, 56 entirely through the flow channels 38, 40, while fluidly coupling the media source 14g (shown in FIG. 1) to the open inlet ports 46, 50 and fluidly coupling the waste destination 14i (shown in FIG. 1) to the open outlet ports 48, 52. As a result, under the fluid pressure applied by the pump assembly 18 (shown in FIG. 1), priming media 23 flows along the flow path 54 from the media source 14g, through the open inlet port 46, through the upper flow channel 38, through the open outlet port 48, and to the waste destination 14i, and priming media 23 flows along the flow path 56 from the media source 14g, through the open inlet port 50, through the lower flow channel 40, through the open outlet port 52, and to the waste destination 14i. Thus, any air within the flow channels 38, 40 will be completely displaced (i.e., flushed out) by the priming media 23. In this manner, when the flow channels 38, 40 are pressurized, the flow paths of fluid within the bioreactor 12a will be consistent and well-defined.

As another example, the valve assembly 16 may also be operated to open the inlet port 46 associated with the upper flow channel 38 and the outlet port 52 associated with the lower flow channel 40, and to close the inlet port 50 associated with the lower flow channel 40 and the outlet port 48 associated with the upper flow channel 38, thereby defining a flow path 58 between the flow channels 38, 40, while fluidly coupling the biological cell source 14a and/or microbead source 14b (shown in FIG. 1) to the open inlet port 46 and fluidly coupling the waste destination 14i (shown in FIG. 1) to the open outlet port 52. As a result, under the fluid pressure applied by the pump assembly 18 (shown in FIG. 1), seeding media 24 containing a seed population of biological cells 2 and/or microbeads 4 flows along the flow path 58 from the biological cell source 14a and/or microbead source 14b, through the open inlet port 46, and into the upper flow channel 38; is then perfused through the porous membrane 36 from the upper flow channel 38 into the lower flow channel 40 (due to the pressure buildup in the upper flow channel 38 caused by the closed outlet port 48 that creates a pressure gradient between the flow channels 38, 40) while the porous membrane 36 captures (filters) the biological cells 2 and/or microbeads 4, thereby seeding the upper flow channel 38 with the population of biological cells 2, and in particular, urging the biological cells 2 against the cell culture surface 44 on the porous membrane 36 via the action of convection; and then flowed from the lower flow channel 40, through the outlet port 52, and to the waste destination 14i.

Significantly, because the biological cells 2 are filtered and captured by the porous membrane 36 while the seeding media 24 perfuses through the porous membrane 36, loss of biological cells 2 and/or microbeads 4 out of the bioreactor 12a is minimized during seeding of the bioreactor 12a with the population of biological cells 2 and/or microbeads 4. In this manner, seeding of the bioreactor 12a with a population of biological cells 2 and/or microbeads 4 may be accomplished robustly and expeditiously without concern that some of the biological cells 2 and/or microbeads 4 will be lost with the outflow of seeding media 24 from the bioreactor 12a. It should be appreciated that, once flow of the seeding media 24 ceases, such that there is no perfusion through the porous membrane 36, the biological cells 2 and/or microbeads 4 will continue to be urged against the cell culture surface 44 of the membrane via the force of gravity. Although, in an alternative embodiment, the seeding media 24 with the biological cells 2 and/or microbeads 4 may be flowed entirely through the upper flow channel 38, e.g., by opening both the inlet port 46 and outlet port 48 of the upper flow channel 38 to define the flow path 54 entirely through the upper flow channel 38, care must be taken to ensure that a significant amount of biological cells 2 and/or microbeads 4 do not escape out of the bioreactor 12a with the outflow of the seeding media 24.

As still another example, the valve assembly 16 may also be operated to open the inlet port 50 and outlet port 52 associated with the lower flow channel 40, and closing the inlet port 46 and outlet port 48 associated with the upper flow channel 38, thereby defining the flow path 56 entirely through the lower flow channel 40, while fluidly coupling the nutrient source 14c, reagent source 14d, and gas source 14e (shown in FIG. 1) to the open inlet port 50 and fluidly coupling the waste destination 14i (shown in FIG. 1) to the open outlet port 52. As a result, under the fluid pressure applied by the pump assembly 18 (shown in FIG. 1), fresh culture media 26 containing nutrient, reagent, and gas flows along the flow path 56 from the nutrient source 14c, reagent source 14d, and gas source 14e, through the open inlet port 50, through the lower flow channel 40, through the outlet port 52 as spent culture media 26′, and to the waste destination 14i, thereby delivering nutrient, reagent, and gas to the environment surrounding the population of biological cells 2 residing in the bioreactor 12a, as well as extracting waste from the environment surrounding the population of biological cells 2 residing in the bioreactor 12a, and in particular, by allowing the nutrient, reagent, and gas within the fresh culture media 26 to diffuse through the porous membrane 36 from the lower flow channel 40 into the upper flow channel 38, and allowing the waste to diffuse through the porous membrane 36 from the upper flow channel 38 into the lower flow channel 40, to facilitate expansion of the population of biological cells 2.

As yet another example, the valve assembly 16 may be also operated to open the inlet port 46 associated with the upper flow channel 38 and the outlet port 52 associated with the lower flow channel 40, and to close the inlet port 50 associated with the lower flow channel 40 and the outlet port 48 associated with the upper flow channel 38, thereby defining the flow path 58 between the flow channels 38, 40 described above, while fluidly coupling the wash buffer source 14f (shown in FIG. 1) to the open inlet port 46 and fluidly coupling the waste destination 14i (shown in FIG. 1) to the open outlet port 52. As a result, under the fluid pressure applied by the pump assembly 18 (shown in FIG. 1), fresh wash buffer 28 flows along the flow path 58 from the biological cell source 14a and/or microbead source 14b, through the open inlet port 46, and into the upper flow channel 38; is then perfused through the porous membrane 36 from the upper flow channel 38 into the lower flow channel 40 (due to the pressure buildup in the upper flow channel 38 caused by the closed outlet port 48 that creates a pressure gradient between the flow channels 38, 40) as used wash buffer 28′ with undesirable material (e.g., unbound molecules, debris, dead cells, or other unwanted molecules) while the porous membrane 36 captures (filters) the biological cells 2 and/or microbeads 4, thereby retaining the biological cells 2 and/or microbeads 4 within the upper flow channel 38; and then flowed from the lower flow channel 40, through the outlet port 52, and to the waste destination 14i. In the same manner that the perfusion of the seeding media 24 with the biological cells 2 and/or microbeads 4 through the porous membrane 36 minimizes loss of biological cells 2 and/or microbeads 4 from the bioreactor 12a, perfusion of the used wash buffer 28′ containing undesirable material through the porous membrane 36 minimizes loss of the biological cells 2 and/or microbeads 4 out of the bioreactor 12a.

As yet another example, the valve assembly 16 may also be operated to open the inlet port 46 and outlet port 48 associated with the upper flow channel 38, and to close the inlet port 50 and outlet port 52 associated with the lower flow channel 40, thereby defining the flow path 54 entirely through the upper flow channel 38, while fluidly coupling the media source 14g (shown in FIG. 1) to the open inlet port 46 and fluidly coupling the biological cell collection destination 14h (shown in FIG. 1) to the open outlet port 48. As a result, under the fluid pressure applied by the pump assembly 18 (shown in FIG. 1), harvesting media 30 containing no biological cells 2 and microbeads 4 flows along the flow path 54 from the media source 14g, through the open inlet port 46, and into the upper flow channel 38; suspending the expanded population of biological cells 2 and microbeads 4 within the harvesting media 30; and flowing the harvesting media 30′ with the expanded population of biological cells 2 and microbeads 4, through the outlet port 48, and to the biological cell collection destination 14h, thereby harvesting the expanded population of biological cells 2 from the upper flow channel 38.

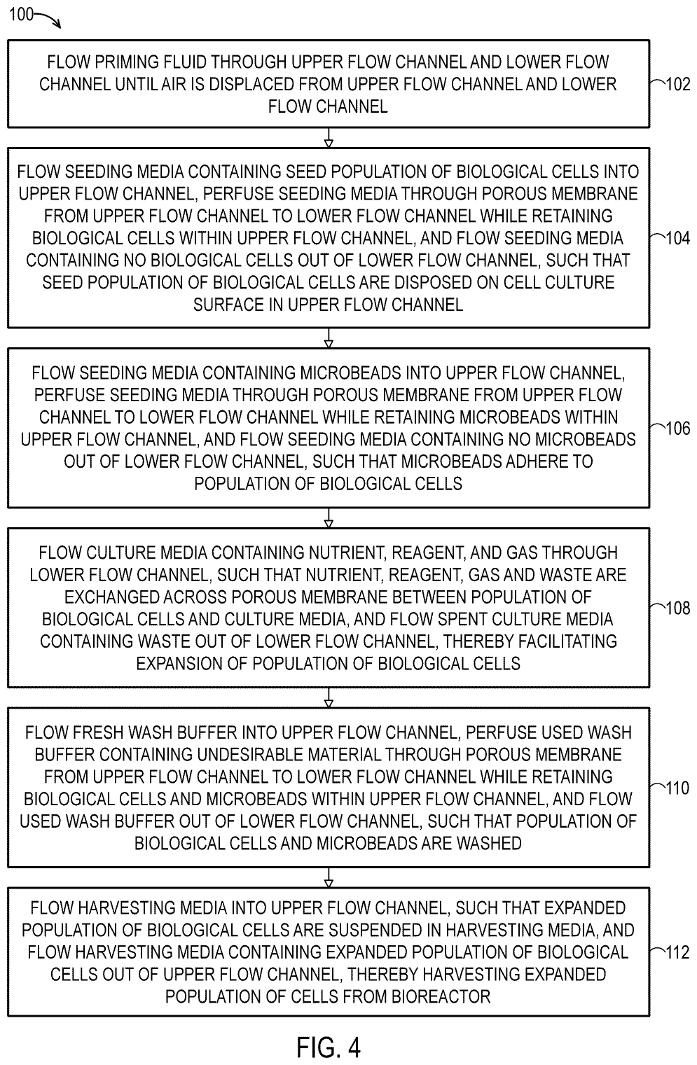

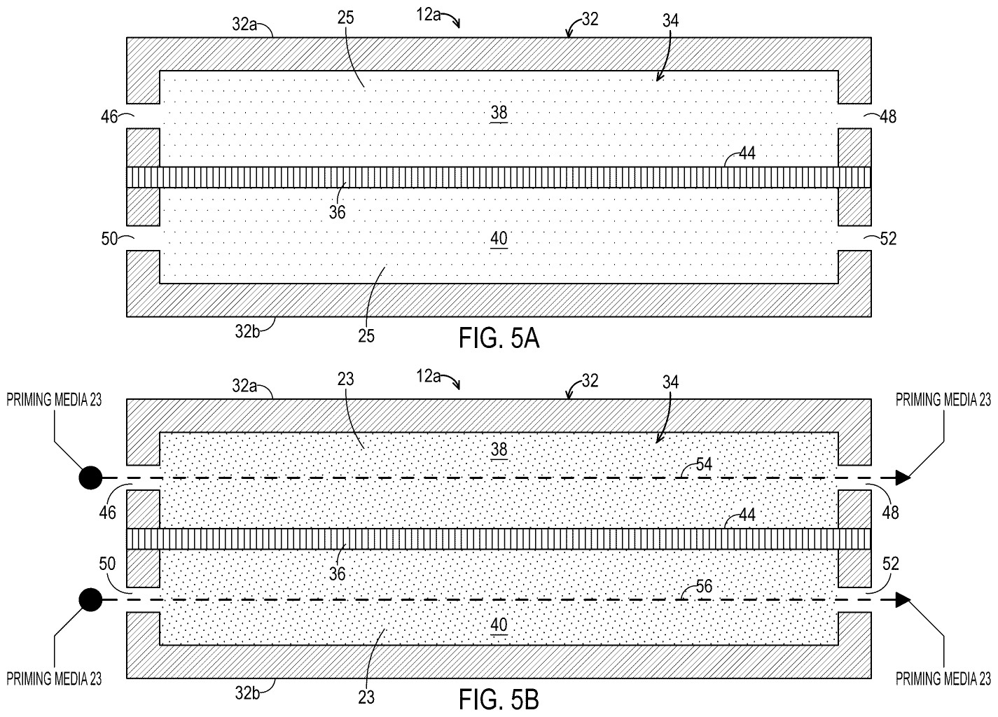

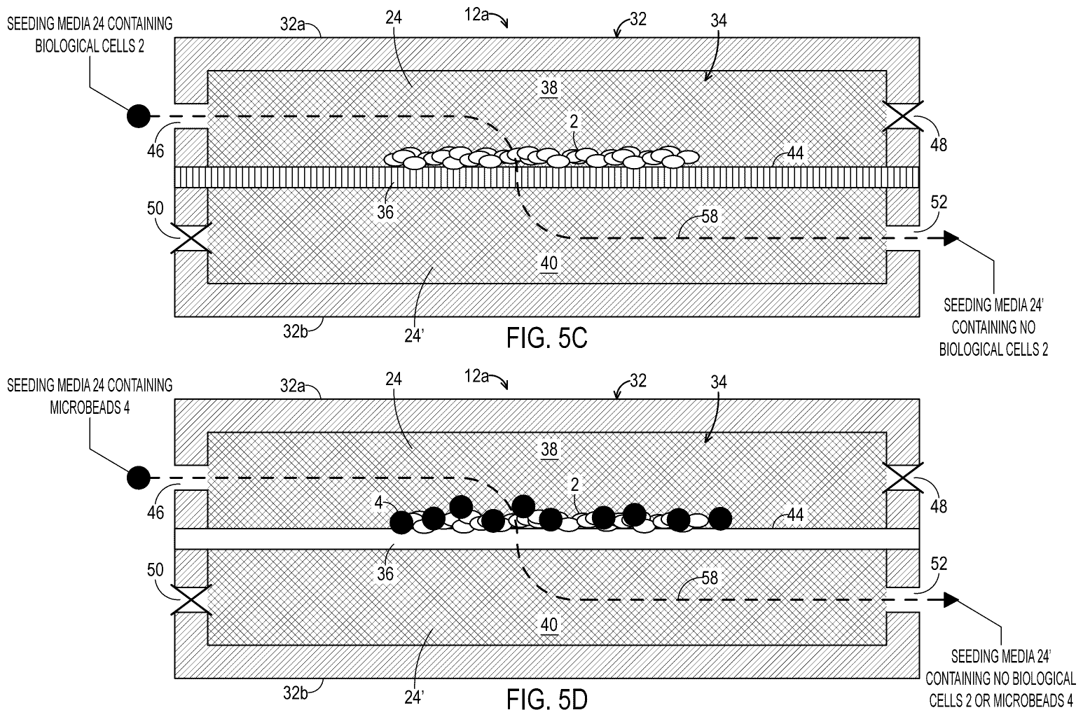

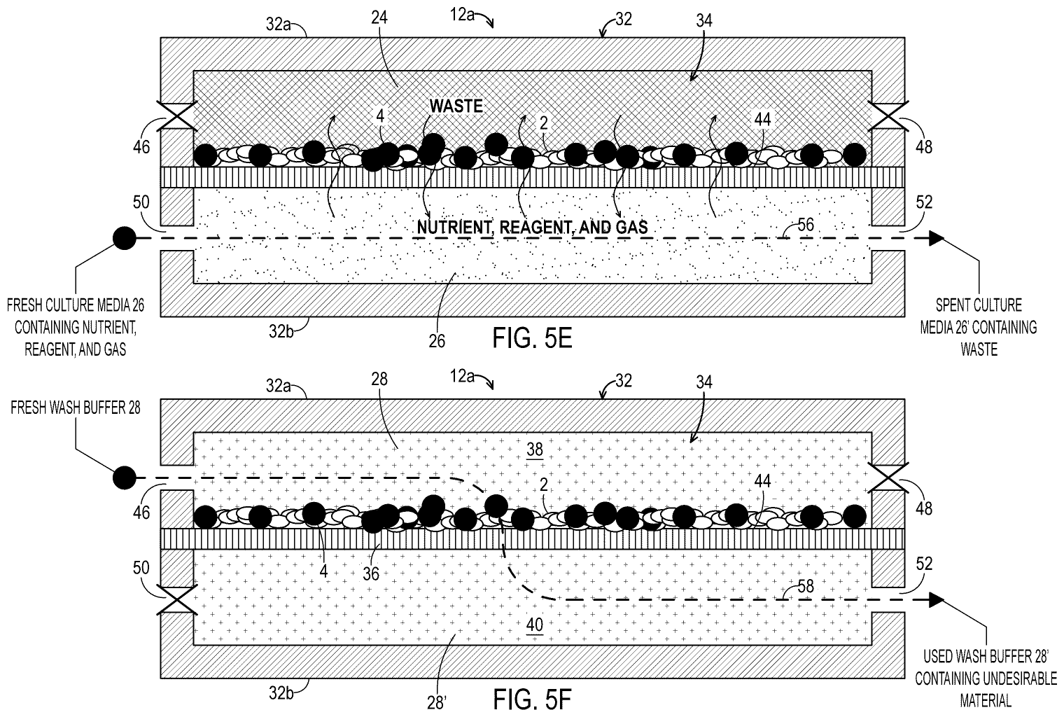

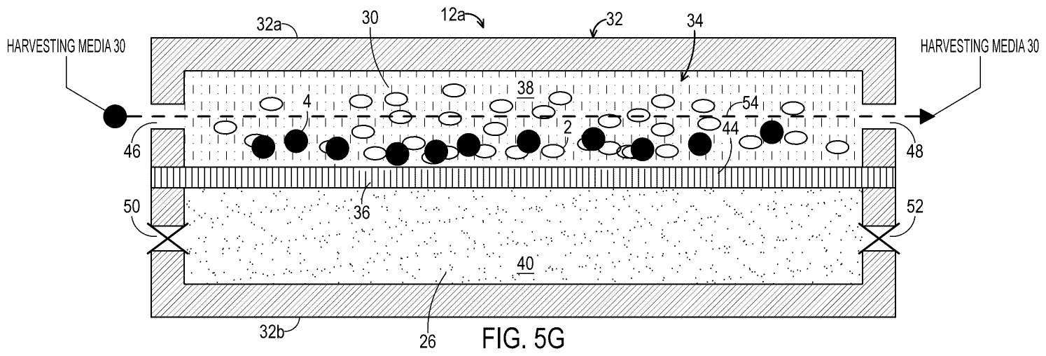

Having described the structure and function of the bioreactor 12a, one method 100 of operating the bioreactor 12a to expand and harvest a population of biological cells 2 (e.g., T lymphocytes (T-cells)) will now be described with reference to FIGS. 4 and 5A-5G.

First, air 25 is flushed out of the flow channels 38, 40. Preferably, as much air as possible, but not necessarily all of the air 25, is flushed out of the flow channels 38, 40. In particular, the flow channels 38, 40 are primed with priming media 23 by flowing the priming media 23 through both the upper flow channel 38 and lower flow channel 40 until the air 25 (see FIG. 5A) is displaced (i.e., flushed out) from the flow channels 38, 40 (step 102) (see FIG. 5B).

In one embodiment, the priming media 23 is concurrently flowed through the upper flow channel 38 and lower flow channel 40. As one example, while the inlet port 46 and outlet port 48 associated with the upper flow channel 38 are open, and the inlet port 50 and outlet port 52 associated with the lower flow channel 40 are open, thereby defining the aforementioned flow path 54 entirely through the upper flow channel 38, and the aforementioned flow path 56 entirely through the lower flow channel 40, the priming media 23 is flowed along the flow path 54 in through the inlet port 46, entirely through the upper flow channel 38, out through the outlet port 48, and the priming media 23 is flowed along the flow path 56 in through the inlet port 50, entirely through the lower flow channel 40, and out through the outlet port 52.

In another embodiment, the priming media 23 is sequentially flowed through the upper flow channel 38 and the lower flow channel 40. As one example, while the inlet port 46 and outlet port 48 associated with the upper flow channel 38 are open, and the inlet port 50 and outlet port 52 associated with the lower flow channel 40 are closed, thereby defining only the flow path 54 entirely through the upper flow channel 38, the priming media 23 is flowed along the flow path 54 in through the inlet port 46, entirely through the upper flow channel 38, and out through the outlet port 48. While the inlet port 46 and outlet port 48 associated with the upper flow channel 38 are closed, and the inlet port 50 and outlet port 52 associated with the lower flow channel 40 are open, thereby defining only the flow path 56 entirely through the lower flow channel 40, the priming media 23 is flowed along the flow path 56 in through the inlet port 50, entirely through the lower flow channel 40, and out through the outlet port 52.

Next, the upper flow channel 38 of the bioreactor 12a is seeded with the population of biological cells 2, such that the population of biological cells 2 rest on the cell culture surface 44 within the upper flow channel 38. In particular, the seeding media 24 with the biological cells 2 is flowed into the upper flow channel 38, and perfused through the porous membrane 36 from the upper flow channel 38 into the lower flow channel 40 while the biological cells 2 are retained within the upper flow channel 38, and then the seeding media 24′ without the biological cells 2 is then flowed out of the lower flow channel 40, thereby disposing a seed population of biological cells 2 on the cell culture surface 44 of the porous membrane 36 via the action of convection (step 104) (see FIG. 5C).

As one example, while the inlet port 46 associated with the upper flow channel 38 and the outlet port 52 associated with the lower flow channel 40 are open, and the inlet port 50 associated with the lower flow channel 40 and the outlet port 48 associated with the upper flow channel 38 are closed, thereby defining only the aforementioned flow path 58 between the flow channels 38, 40, the seeding media 24 with the biological cells 2 is flowed along the flow path 58 in through the inlet port 46 and into the upper flow channel 38, and perfused through the porous membrane 36 from the upper flow channel 38 to the lower flow channel 40 while the porous membrane 36 captures (filters) the biological cells 2, and then the seeding media 24′ without the biological cells 2 is further flowed along the flow path 58 through the lower flow channel 40 and out through the outlet port 52.

Optionally, microbeads 4 are disposed within the upper flow channel 38, such that the population of biological cells 2 adhere to the microbeads 4. In particular, the seeding media 24 with the microbeads 4 is flowed into the upper flow channel 38, perfused through the porous membrane 36 from the upper flow channel 38 into the lower flow channel 40 while the microbeads 4 are retained within the upper flow channel 38, and then the seeding media 24′ without the microbeads 4 is flowed out of the lower flow channel 40, such that microbeads 4 adhere to population of biological cells 2 (step 106) (see FIG. 5D).

As one example, while the inlet port 46 associated with the upper flow channel 38 and the outlet port 52 associated with the lower flow channel 40 are open, and the inlet port 50 associated with the lower flow channel 40 and the outlet port 48 associated with the upper flow channel 38 are closed, thereby defining only the flow path 58 between the flow channels 38, 40, the seeding media 24 with the microbeads 4 is flowed along the flow path 58 in through the inlet port 46 and into the upper flow channel 38, perfused through the porous membrane 36 from the upper flow channel 38 to the lower flow channel 40 while the porous membrane 36 captures (filters) the microbeads 4, and then the seeding media 24′ without the microbeads 4 is flowed further along the flow path 58 through the lower flow channel 40 and out through the outlet port 52.

While the microbeads 4 have been described as being delivered separately from the biological cells 2, into the upper flow channel 38, it should be appreciated that the microbeads 4 may be delivered with the biological cells 2, into the upper flow channel 38.

Next, nutrient, reagent, and gas are delivered to the population of biological cells 2, and waste is extracted from the population of biological cells 2, thereby facilitating the expansion of the population of biological cells 2 within the upper flow channel 38. In particular, the fresh culture media 26 is flowed (continually or intermittently) into the lower flow channel 40, such that nutrient, reagent, gas, and waste are exchanged across the porous membrane 36 between the population of biological cells 2 and culture media 26 (i.e., the nutrient, reagent, and gas are provided to, while the waste is extracted from, the population of biological cells 2, 25 with the nutrient, reagent, and gas diffusing through the porous membrane 36 from the culture media 26 in the lower flow channel 40 to the seeding media 24 in the upper flow channel 38, and the waste diffusing through the porous membrane 36 from the seeding media 24 in the upper flow channel 38 to the culture media 26 in the lower flow channel 40), and then the spent culture media 26′ with the waste is flowed out of the lower flow channel 40, thereby facilitating expansion of the population of biological cells 2 (step 108) (see FIG. 5E). Significantly, diffusion of the nutrient, reagent, gas, and waste through the porous membrane 36 occurs while allowing the biological cells 2 and microbeads 4 to continue to stably rest on the cell culture surface 44.

As one example, while the inlet port 50 and outlet port 52 associated with the lower flow channel 40 are open, and the inlet port 46 and outlet port 48 associated with the upper flow channel 38 are closed, thereby defining only the flow path 56 entirely through the lower flow channel 40, the fresh culture media 26 containing the nutrient, reagent, and gas is flowed along the flow path 56 in through the inlet port 50 and into the lower flow channel 40, and then the spent culture media 26′ with the waste is flowed further along the flow path 56 through the lower flow channel 40 and out through the outlet port 52.

It should be appreciated that, over a period of time if the culture media 26 is not replaced within the lower flow channel 40 (either continuously or intermittently), the seeding media 24 within the upper flow channel 38 and the culture media 26 contained in the lower flow channel 40 will become a homogenous solution with diffusion of nutrient, gas, reagents, and waste occurring across the porous membrane 36 between the flow channels 38, 40 ceasing upon achieving homogeneity between the seeding media 24 and the culture media 26. Thus, it is preferred that culture media 26 be replaced within the lower flow channel 40 with fresh culture media 26 before the culture media 26 becomes completely spent; i.e., prior to the seeding media 24 and the culture media 26 achieving homogeneity. Thus, the continual or intermittent flow of culture media 26 will maintain the chemical gradient between the flow channels 38, 40 necessary to effect the diffusion of nutrient, gas reagent, and waste through the porous membrane 36 (i.e., will maintain the concentration of nutrient, reagent, and gas in the culture media 26 at a higher level than the previously diffused nutrient, reagent, and gas remaining in the upper flow channel 38, and will maintain the concentration of previously diffused waste in the culture media 26 at a lower level than the remaining waste in the upper flow channel 38).

Anytime during incubation, the population of biological cells 2 may be washed. In particular, much like the seeding of the upper flow channel 38 with the population of biological cells 2 with or without the microbeads 4, the fresh wash buffer 28 is flowed into the upper flow channel 38, and used wash buffer 28′ containing undesirable material (e.g., unbound molecules, debris, dead cells, or other unwanted molecules) is perfused through the porous membrane 36 from the upper flow channel 38 into the lower flow channel 40 while the biological cells 2 and microbeads 4 are retained within the upper flow channel 38, and then flowed out of the lower flow channel 40, thereby washing the population of biological cells 2 and microbeads 4 (step 110) (see FIG. 5F).

As one example, while the inlet port 46 associated with the upper flow channel 38 and the outlet port 52 associated with the lower flow channel 40 are open, and the inlet port 50 associated with the lower flow channel 40 and the outlet port 48 associated with the upper flow channel 38 are closed, thereby defining only the flow path 58 between the flow channels 38, 40, the washer buffer 28 is flowed along the flow path 58 in through the inlet port 46 and into the upper flow channel 38, perfused through the porous membrane 36 from the upper flow channel 38 to the lower flow channel 40 while the porous membrane 36 captures (filters) the biological cells 2 and microbeads 4, and used washer buffer 30 with the undesirable material is flowed further along the flow path 58 through the lower flow channel 40 and out through the outlet port 52.

In an alternative method, the fresh wash buffer 28 may be flowed (continually or intermittently) into the lower flow channel 40, such that the active components of the fresh wash buffer 28 diffuse through the porous membrane 36 from the fresh wash buffer 28 in the lower flow channel 40 to the media in the upper flow channel 38. The media with the undesirable material may be then flowed out of the upper flow channel 38.

As one example, while the inlet port 50 and outlet port 52 associated with the lower flow channel 40 are open, and the inlet port 46 and outlet port 48 associated with the upper flow channel 38 are closed, thereby defining only the flow path 56 entirely through the lower flow channel 40, the fresh wash buffer 28 may be flowed along the flow path 56 in through the inlet port 50 and into the lower flow channel 40, and then the used wash buffer 28′ without the active components (or significantly less active components) is flowed further along the flow path 56 through the lower flow channel 40 and out through the outlet port 52. Then, while the inlet port 46 and outlet port 48 associated with the upper flow channel 38 are open, and the inlet port 50 and outlet port 52 associated with the lower flow channel 40 are closed, thereby defining only the flow path 54 entirely through the upper flow channel 38, media (e.g., PBS) may be flowed along the flow path 54 in through the inlet port 46, through the upper flow channel 38, and out through the outlet port 48, thereby removing the media with the undesirable material from the upper flow channel 38. It should be appreciated that in this alternative method, the media must be flowed through the upper flow channel 38 at a relatively low rate, such that the biological cells 2 and/or microbeads 4 are not suspended and lost out of the upper flow channel 38.

Notably, the washing step, can be performed without mechanically agitating the upper flow channel 38, since the population of biological cells 2 do not have to be adhered by structural functionalization of the cell culture surface 44 to withstand high tangential flow rates through the upper flow channel 38. In an optional method, the upper flow channel 38 may be slightly agitated to suspend the population of biological cells 2 and microbeads 4 within the seeding media 24 immediately prior to, or as a part of, the washing step, so that the biological cells 2 do not hinder the perfusion of the wash buffer 19 through the porous membrane 36. For example, washer buffer 28 or other media may be gently flowed back and forth through the upper flow channel 38 via the pump assembly 18 until all of the biological cells 2 and microbeads 4 are suspended population of biological cells 2 and microbeads 4. The washing step can be performed several times.

The culture media 26 can then be again flowed through the lower flow channel 40 for exchange of the nutrient, gas, reagent, and waste between the population of biological cells 2 and the culture media 26 (step 108).

When ready to be harvested, the expanded population of biological cells 2 are harvested from the upper flow channel 38.

In particular, harvesting media 30 is flowed into the upper flow channel 38, such that the expanded population of biological cells 2 and microbeads 4 are suspended in the harvesting media 30, and then the harvesting media 30′ with the expanded population of biological cells 2 and microbeads 4 is flowed out of the upper flow channel 38, thereby extracting the expanded population of biological cells 2 from the bioreactor 12a (step 112) (see FIG. 5G).

As one example, while the inlet port 46 and outlet port 48 associated with the upper flow channel 38 are open, and the inlet port 50 and outlet port 52 associated with the lower flow channel 40 are closed, thereby defining only the flow path 54 entirely through the upper flow channel 38, the harvesting media 30 is flowed along the flow path 54 in through the inlet port 46 and into the upper flow channel 38, and then the harvesting media 30′ with the expanded population of biological cells 2 and microbeads 4 is flowed further along the flow path 54 through the upper flow channel 38 and out through the outlet port 48.

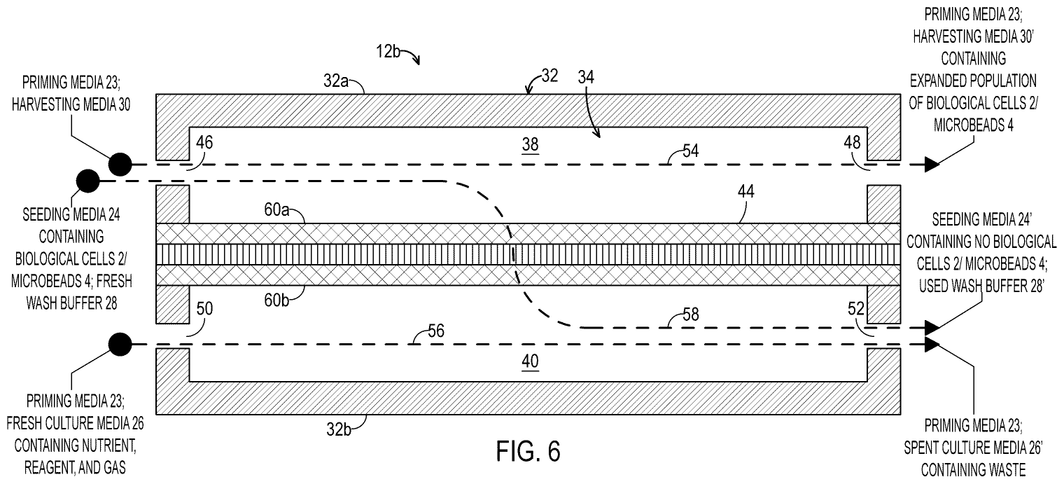

Referring now to FIG. 6, another embodiment of a bioreactor 12b for use in the bioreactor system 10 of FIG. 1 will now described. The bioreactor 12b is similar to the bioreactor 12a illustrated in FIGS. 2-3, with the exception that the bioreactor 12b additionally comprises at least one permeable support structure 54 (two permeable support structures 60a, 60b shown) configured for reducing a lateral flex of the porous membrane 36 in response to a pressure differential between the flow channels 38, 40. It should be noted that excessive lateral flexing of the porous membrane 36 creates a shear force that may disturb the population of biological cells 2 resting on the top surface of the porous membrane 36, resulting in less expansion of the population of biological cells 2. Furthermore, excessive lateral flexing of the porous membrane 36 may damage the porous membrane 36.

In the illustrated embodiment, an upper permeable support structure 60a is affixed between the upper housing portion 32a and the top surface of the porous membrane 36 (e.g., via heat staking, thermal or ultrasonic welding, laminating, or simply being pinched in an interference fit) to reduce or minimize upward lateral flexing of the porous membrane 36 into the upper flow channel 38 in response to a pressure differential resulting from the flow of culture media 26 through the lower flow channel 40 (i.e., the fluid pressure in the lower flow channel 40 becomes higher than the fluid pressure in the upper flow channel 38). A lower permeable support structure 60b is affixed between the lower housing portion 32b and the bottom surface of the porous membrane 36 (e.g., via heat staking, thermal or ultrasonic welding, laminating, or simply being pinched in an interference fit) to reduce or minimize downward lateral flexing of the porous membrane 36 into the lower flow channel 40 in response to a pressure differential resulting from the flow of seeding media 24 or wash buffer 28 within the upper flow channel 38 (i.e., the fluid pressure in the upper flow channel 38 becomes higher than the fluid pressure in the lower flow channel 40).

In the embodiment illustrated in FIG. 6, each of the permeable support structures 60a, 60b is highly porous so as to not unduly restrict the diffusion of nutrient, gas, reagent, and waste through the porous membrane 36 between the upper flow channel 38 and the lower flow channel 40. For example, each of the permeable support structures 60a, 60b may comprise a woven mesh (e.g., composed stainless steel or a polymer, such as, e.g., polycarbonate, polystryrene, COC, COP, polypropylene, etc.). Each of the support structures 60a, 60b may have a suitable thickness, e.g., in the range of 0.1 mm-2 mm, and a high porosity (high enough to allow perfusion of liquid without compromising the structure of the support structures 60a, 60b to the extent that the support structures 60a, 60b flex in response to the anticipated pressure differential between the flow channels 38, 40), e.g., in the range of 10%-90%. In one specific embodiment, each of the support structures 60a, 60b has a thickness of 1 mm and a porosity of 5%. Although the bioreactor 12b is described and illustrated as comprising two permeable support structures 60a, 60b, the bioreactor 12b may alternatively comprise only one membrane 36 and a single support structure, e.g., if it is anticipated that any significant pressure differential between the flow channels 38, 40 will occur in only one direction.

The bioreactor 12b may be operated to expand and harvest a population of biological cells 2 (e.g., T lymphocytes (T-cells) is in the same as the bioreactor 12a described above with respect to FIGS. 4 and 5A-5G, with the exception that the permeable support structures 60a, 60b minimize the lateral flex of the porous membrane 36 during significant pressure differentials between the upper flow channel 38 and the lower flow channel 40.

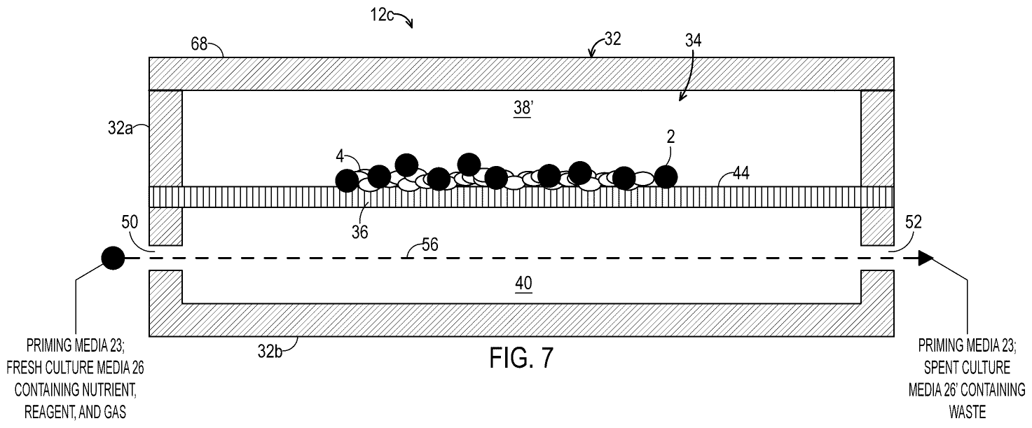

Referring now to FIG. 7, still another embodiment of a bioreactor 12c for use in the bioreactor system 10 of FIG. 1 will now described. The bioreactor 12c is similar to the bioreactor 12a illustrated in FIGS. 2-3, with the exception that, instead of having a dynamic chamber in the form of an upper flow channel, the bioreactor 12e comprises an upper static chamber 38′. Thus, the bioreactor 12c does not comprise any inlet or outlet ports for tangentially flowing the seeding media 24 or wash buffer in and out of bioreactor 12e. Rather, seeding media 24 and microbeads may be introduced into the upper static chamber 38′ by removing a lid 68 affixed to the upper static chamber 38′, e.g., via a threaded arrangement, or by puncturing one or more septums (not shown) at the top of the static 38′ with a syringe (not shown) for introducing or removing the seeding media 24 or wash buffer into and out of the upper static chamber 38′. It should be appreciated that the upper flow channel of the bioreactor 12b illustrated in FIG. 6 may be similarly replaced with a static first flow channel.

The bioreactor 12c may be operated to expand and harvest a population of biological cells 2 (e.g., T lymphocytes (T-cells) is in the same as the bioreactor 12a described above with respect to FIGS. 4 and 5A-5G, with the exception that the bioreactor 12c is not seeded with the population of biological cells 2 or microbeads 4 by dynamically flowing seeding media 24, but rather are statically introduced into the upper static chamber 38′, and the expanded population of biological cells 2 are not harvested from the bioreactor 12c by dynamically flowing harvesting media 30, but rather are statically removed from the upper static chamber 38′. In this embodiment, fresh culture media 26 containing nutrient, reagent, and gas can be flowed (continuously or intermittently) through the upper flow channel 38, thereby providing nutrient, reagent, and gas to, and extracting waste from, the population of biological cells 2 residing in the lower flow channel 40 via the action of diffusion through the porous membrane 36, thereby facilitating expansion of the population of biological cells 2.

The valve assembly 16 (illustrated in FIG. 1) may be operated to open the inlet port 50 and outlet port 52 associated with the lower flow channel 40, thereby defining the flow paths 56 entirely through the lower flow channel 40, while fluidly coupling the media source 14g (shown in FIG. 1) to the open inlet port 50 and fluidly coupling the waste destination 14i (shown in FIG. 1) to the open outlet ports 48, 52. As a result, under the fluid pressure applied by the pump assembly 18 (shown in FIG. 1), priming media 23 flows along the flow path 56 from the media source 14g, through the open inlet port 50, through the lower flow channel 40, through the open outlet port 52, and to the waste destination 14i. Thus, any air within the lower flow channels 40 will be completely displaced (i.e., flushed out) by the priming media 23. In this manner, when the lower flow channel 40 is pressurized, the flow path 56 of the culture media 26 will be consistent and well-defined.

The valve assembly 16 may also be operated to open the inlet port 50 and outlet port 52 associated with the lower flow channel 40, thereby defining the flow path 56 entirely through the lower flow channel 40, while fluidly coupling the nutrient source 14c, reagent source 14d, and gas source 14e (shown in FIG. 1) to the open inlet port 50 and fluidly coupling the waste destination 14i (shown in FIG. 1) to the open outlet port 52. As a result, under the fluid pressure applied by the pump assembly 18 (shown in FIG. 1), fresh culture media 26 containing nutrient, reagent, and gas flows along the flow path 56 from the nutrient source 14c, reagent source 14d, and gas source 14e, through the open inlet port 50, through the lower flow channel 40, through the outlet port 52 as spent culture media 26′, and to the waste destination 14i, thereby delivering nutrient, reagent, and gas to the environment surrounding the population of biological cells 2 residing in the bioreactor 12a, as well as extracting waste from the environment surrounding the population of biological cells 2 residing in the bioreactor 12a, and in particular, by allowing the nutrient, reagent, and gas within the fresh culture media 26 to diffuse through the porous membrane 36 from the lower flow channel 40 into the upper static chamber 38′, and allowing the waste to diffuse through the porous membrane 36 from the upper static chamber 38′ into the lower flow channel 40, to facilitate expansion of the population of biological cells 2.

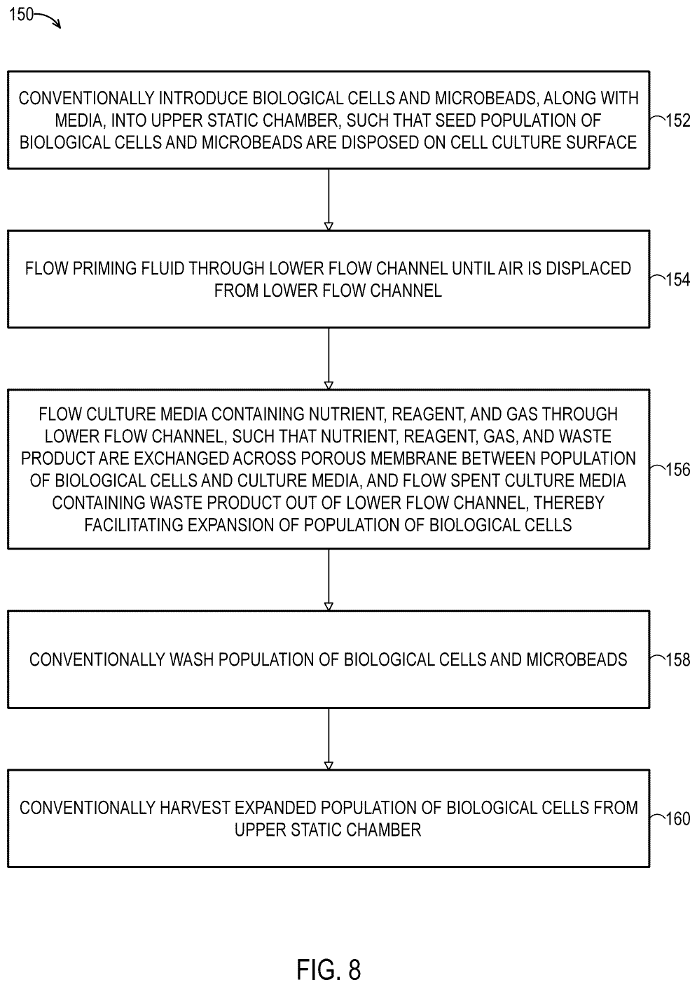

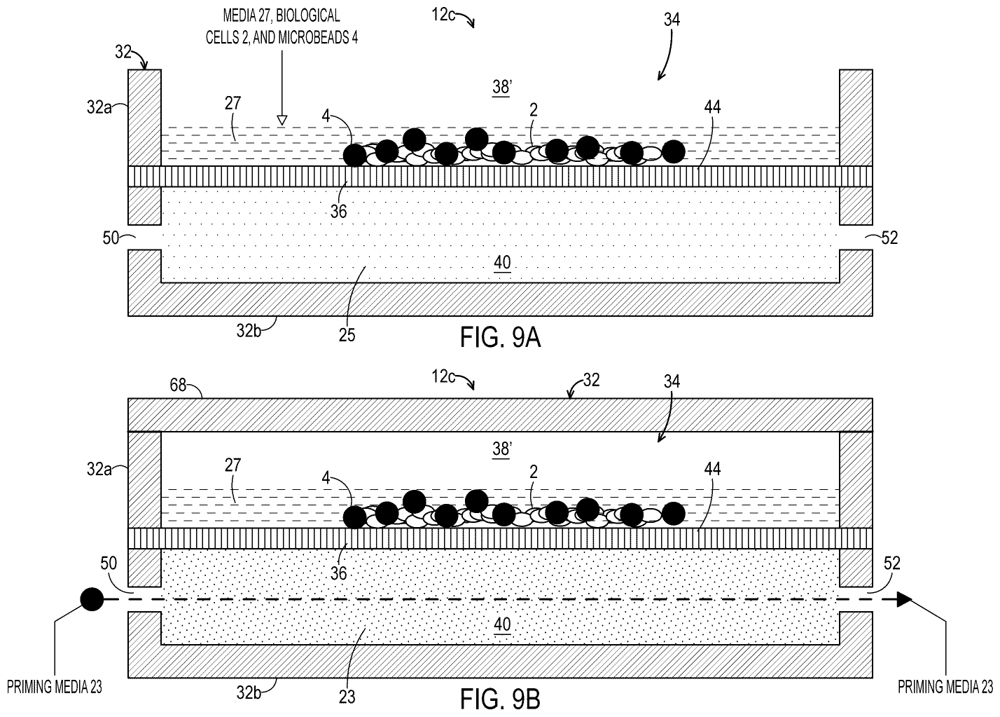

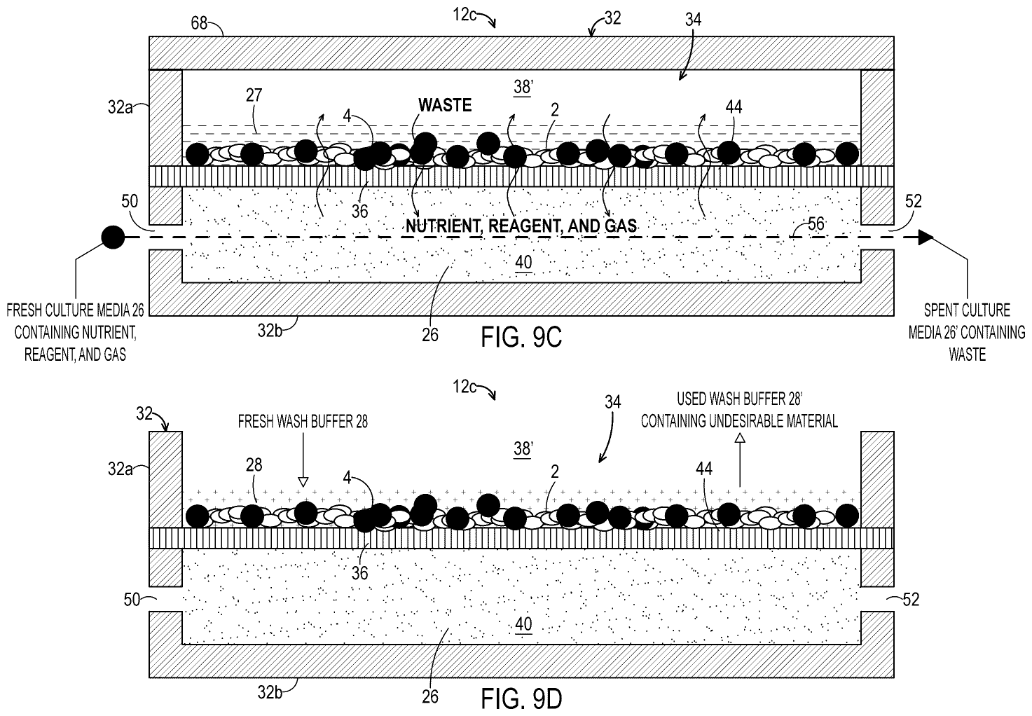

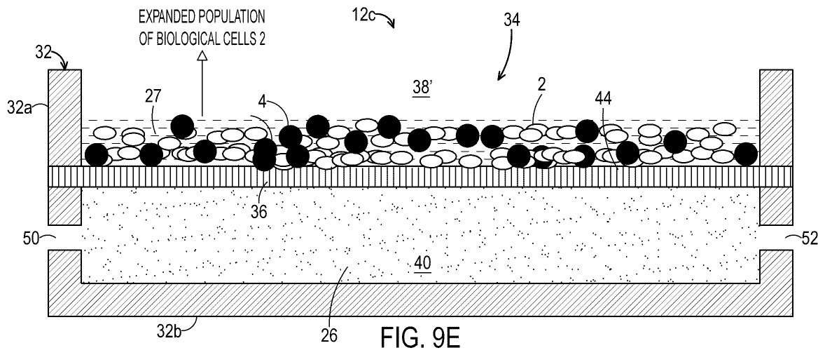

One method 150 of operating the bioreactor 12c to expand and harvest a population of biological cells 2 (e.g., T lymphocytes (T-cells)) will now be described with reference to FIGS. 8 and 9A-9E.

First, biological cells 2 and optional microbeads 4, along with media 27 (e.g., PBS or cell culture media), are conventionally introduced into the upper static chamber 38′, such that a seed population of biological cells 2 and microbeads 4 are disposed on the cell culture surface 44 of the porous membrane 36 (step 152) (see FIG. 9A). As one example, the upper static chamber 38′ may be opened (e.g., by removing the lid 68), the biological cells 2 and optional microbeads 4 may be introduced into the upper static chamber 38′ with a syringe, and the upper static chamber 38′ may be closed (e.g., by reinstalling the lid 68). In one embodiment, the upper static chamber 38 need not be completely filled with the media 27. As such, the amount of nutrient, reagent, and gas contained within the culture media 18 that flows through the lower flow channel 40 may also be minimized, since less nutrient, reagent, and gas will be required to diffuse across the porous membrane 36 to maintain the environment necessary to promote the expansion of the population of biological cells 2.

Next, air 25 is flushed out of the lower flow channel 40. Preferably, as much air 25 as possible, but not necessarily all of the air 25, is flushed out of the lower flow channel 40. In particular, the flow channel 40 is primed with priming media 23 by flowing the priming media 23 through the lower flow channel 40 until the air 25 (see FIG. 9A) is displaced (i.e., flushed out) from the lower flow channel 40 (step 154) (see FIG. 9B). As one example, while the inlet port 50 and outlet port 52 associated with the lower flow channel 40 are open, thereby defining the flow path 56 entirely the lower flow channel 40, the priming media 23 is flowed along the flow path 56 in through the inlet port 50, entirely through the lower flow channel 40, and out through the outlet port 52.

Next, nutrient, reagent, and gas are delivered to the population of biological cells 2, and waste is extracted from the population of biological cells 2, thereby facilitating the expansion of the population of biological cells 2 within the upper static chamber 38. In particular, the fresh culture media 26 is flowed (continually or intermittently) into the lower flow channel 40, such that nutrient, reagent, gas, and waste are exchanged across the porous membrane 36 between the population of biological cells 2 and culture media 26 (i.e., the nutrient, reagent, and gas are provided to, while the waste is extracted from, the population of biological cells 2, with the nutrient, reagent, and gas diffusing through the porous membrane 36 from the culture media 26 in the lower flow channel 40 to the media 27 in the upper static chamber 38, and the waste diffusing through the porous membrane 36 from the media in the upper static chamber 38 to the culture media 26 in the lower flow channel 40), and then the spent culture media 26′ with the waste is flowed out of the lower flow channel 40, thereby facilitating expansion of the population of biological cells 2 (step 156) (see FIG. 9C). Significantly, diffusion of the nutrient, reagent, gas, and waste through the porous membrane 36 occurs while allowing the biological cells 2 and microbeads 4 to continue to stably rest on the cell culture surface 44.

As one example, while the inlet port 50 and outlet port 52 associated with the lower flow channel 40 are open, thereby defining the flow path 56 entirely through the lower flow channel 40, the fresh culture media 26 containing the nutrient, reagent, and gas is flowed along the flow path 56 in through the inlet port 50 and into the lower flow channel 40, and then the spent culture media 26′ with the waste is flowed further along the flow path 56 through the lower flow channel 40 and out through the outlet port 52. For the same reasons discussed above with respect to the method 100 of operating the bioreactor 12a of FIGS. 2-3, it is preferred that culture media 26 be replaced within the lower flow channel 40 with fresh culture media 26 before the culture media 26 becomes completely spent; i.e., prior to the media 27 and the culture media 26 achieving homogeneity.

Anytime during incubation, the population of biological cells 2 and microbeads 4 may be conventionally washed (step 158) (see FIG. 9D). As one example, the upper static chamber 38′ may be opened (e.g., by removing the lid 68), the washer buffer 28 may be repeatedly introduced into the upper static chamber 38′ with a syringe to remove the undesirable material (e.g., unbound molecules, debris, dead cells, or other unwanted molecules) from the population of biological cells 2 and microbeads 4, and removed, along with the undesirable material, from the upper static chamber 38′ with a syringe.

The culture media 26 can then be again flowed through the lower flow channel 40 for exchange of the nutrient, gas, reagent, and waste between the population of biological cells 2 and the culture media 26 (step 156). When ready the expanded population of biological cells 2 may be conventionally harvested from the upper static chamber 38′ (step 160) (see FIG. 9E). As one example, the upper static chamber 38′ may be opened (e.g., by removing the lid 68), the biological cells 2 may be removed from the upper static chamber 38′ with a syringe, and the upper static chamber 38′ may be closed (e.g., by reinstalling the lid 68).

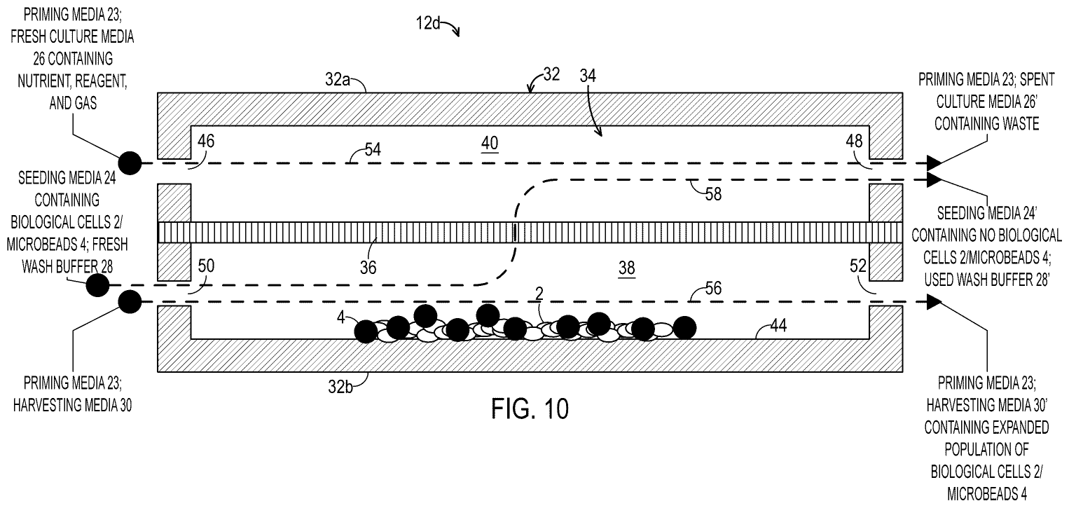

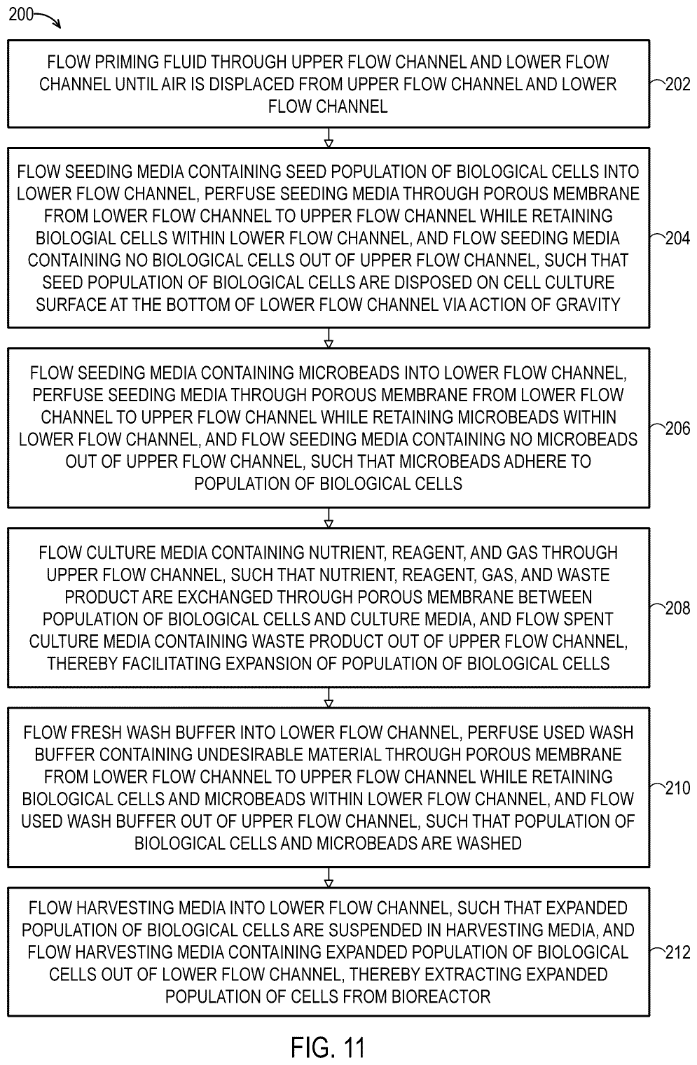

Referring now to FIG. 10, still another embodiment of a bioreactor 12d for use in the bioreactor system 10 of FIG. 1 will now described. The bioreactor 12d is similar to the bioreactor 12a illustrated in FIGS. 2-3, with the exception that the cell culture surface 44 is not formed on the porous membrane 36, but rather is separated and spaced a predetermined distance from the porous membrane 36. In the embodiment illustrated in FIG. 10, the cell culture surface 44 is located in the lower flow channel 40 and formed on a wall of the housing 32, and in particular, on the bottom wall of the housing 32. In this manner, gravity urges the population of biological cells 2 to rest on the cell culture surface 44.

In this embodiment, the seeding media 24 containing the seed population of biological cells 2 (and optionally microbeads 4) can be flowed into the lower flow channel 40 to seed the bioreactor 12d with a population of biological cells 2 (and optionally microbeads 4); fresh culture media 26 containing nutrient, reagent, and gas can be flowed (continuously or intermittently) through the upper flow channel 38, thereby providing nutrient, reagent, and gas to, and extracting waste from, the population of biological cells 2 residing in the lower flow channel 40 via the action of diffusion through the porous membrane 36, thereby facilitating expansion of the population of biological cells 2; wash buffer 28 may be flowed into the lower flow channel 40 during incubation of the population of biological cells 2 to remove undesirable material (e.g., unbound molecules, debris, dead cells, or other unwanted molecules); and harvesting media 30 can be flowed into the lower flow channel 40 to extract the expanded population of biological cells 2 from the bioreactor 12d.

Notably, by locating the cell culture surface 44 on which the population of biological cells 2 rest away from the porous membrane 36, the population of biological cells 2 will not be disturbed by any shear force that would otherwise result from the lateral flexing of the porous membrane 36 in response to a significant pressure differential between the flow channels 38, 40. Since the cell culture surface 44 is located on the rigid wall of the housing 32, a very stable platform on which the population of biological cells 2 rest is provided. It is preferred that the cell culture surface 44 be spaced away from the porous membrane 36 a low or minimal distance (e.g., at least 1 mm), such that the population of biological cells 2 resting on the cell culture surface 44 do not come into contact with the porous membrane 36. Not only does sufficient spacing between the population of biological cells 2 and the porous membrane 36 reduce or minimize any disturbance of the population of biological cells 2 when the porous membrane 36 lateral flexes in response to a significant pressure differential between the flow channels 38, 40, clogging of the pores within the porous membrane 36 with biological cells 2 will be avoided. As such, perfusion of the seeding media 24 and optional wash buffer 28, as well as diffusion of nutrient, reagent, gas, and waste, across the membrane 26 will be more efficient.

Furthermore, to the extent that the porous membrane 36 is not completely impermeable to the biological cells 2, maintaining the population of the biological cells 2 on the cell culture surface 44 away from the porous membrane 36 will prevent any loss (or reduce loss) of biological cells 2 (e.g., via creeping or suction) through the porous membrane 36.

The valve assembly 16 (illustrated in FIG. 1) may be operated to selectively open and close the ports 46-52, such that different flow paths may be defined within the interior cavity 34 of the bioreactor 12d.

As one example, the valve assembly 16 may be operated to open the inlet port 46 and outlet port 48 associated with the upper flow channel 38, and to open the inlet port 50 and outlet port 52 associated with the lower flow channel 40, thereby respectively defining flow paths 54, 56 entirely through the flow channels 38, 40, while fluidly coupling the media source 14g (shown in FIG. 1) to the open inlet ports 46, 50 and fluidly coupling the waste destination 14i (shown in FIG. 1) to the open outlet ports 48, 52. As a result, under the fluid pressure applied by the pump assembly 18 (shown in FIG. 1), priming media 23 flows along the flow path 54 from the media source 14g, through the open inlet port 46, through the upper flow channel 38, through the open outlet port 48, and to the waste destination 14i, and priming media 23 flows along the flow path 56 from the media source 14g, through the open inlet port 50, through the lower flow channel 40, through the open outlet port 52, and to the waste destination 14i. As a result, any air within the flow channels 38, 40 will be completely displaced (i.e., flushed out) by the priming media 23. In this manner, when the flow channels 38, 40 are pressurized, the flow paths of fluid within the bioreactor 12a will be consistent and well-defined.

As another example, the valve assembly 16 may be operated to close the inlet port 46 associated with the upper flow channel 38 and the outlet port 52 associated with the lower flow channel 40, and to open the inlet port 50 associated with the lower flow channel 40 and the outlet port 48 associated with the upper flow channel 38, thereby defining a flow path 58 between the flow channels 38, 40, while fluidly coupling the biological cell source 14a and/or microbead source 14b (shown in FIG. 1) to the open inlet port 50 and fluidly coupling the waste destination 14i (shown in FIG. 1) to the open outlet port 48. As a result, under the fluid pressure applied by the pump assembly 18 (shown in FIG. 1), seeding media 24 with a population of biological cells 2 and/or microbeads 4 flows along the flow path 58 from the biological cell source 14a and/or microbead source 14b, through the open inlet port 50, and into the lower flow channel 40; is then perfused through the porous membrane 36 from the lower flow channel 40 into the upper flow channel 38 (due to the pressure buildup in the lower flow channel 40 caused by the closed outlet port 52 that creates a pressure gradient between the flow channels 38, 40) while the porous membrane 36 captures (filters) the biological cells 2 and/or microbeads 4, thereby seeding the lower flow channel 40 with the population of biological cells 2; and then flowed from the upper flow channel 38, through the outlet port 48, and to the waste destination 14i.

Significantly, because the biological cells 2 are filtered and captured by the porous membrane 36 while the seeding media 24 perfuses through the porous membrane 36, loss of biological cells 2 and/or microbeads 4 out of the bioreactor 12d is minimized during seeding of the bioreactor 12d with the population of biological cells 2 and/or microbeads 4. In this manner, seeding of the bioreactor 12d with a population of biological cells 2 and/or microbeads 4 may be accomplished robustly and expeditiously without concern that some of the biological cells 2 and/or microbeads 4 will be lost with the outflow of seeding media 24 from the bioreactor 12d. It should be appreciated that, once flow of the seeding media 24 ceases, such that there is no perfusion through the porous membrane 36, the biological cells 2 and/or microbeads 4 will come to rest against the cell culture surface 44 on the bottom wall of the housing 32 via the force of gravity. Although, in an alternative embodiment, the seeding media 24 with the biological cells 2 and/or microbeads 4 may be flowed entirely through the lower flow channel 40, e.g., by opening both the inlet port 50 and outlet port 52 of the lower flow channel 38 to define the flow path 56 entirely through the lower flow channel 40, care must be taken to ensure that a significant amount of biological cells 2 and/or microbeads 4 do not escape out of the bioreactor 12d with the outflow of the seeding media 24.

As still another example, the valve assembly 16 may also be operated to open the inlet port 46 and outlet port 48 associated with the upper flow channel 38, and closing the inlet port 50 and outlet port 52 associated with the lower flow channel 40, thereby defining the flow path 54 entirely through the upper flow channel 38, while fluidly coupling the nutrient source 14c, reagent source 14d, and gas source 14e (shown in FIG. 1) to the open inlet port 46 and fluidly coupling the waste destination 14i (shown in FIG. 1) to the open outlet port 48. As a result, under the fluid pressure applied by the pump assembly 18 (shown in FIG. 1), fresh culture media 26 containing nutrient, reagent, and gas flows along the flow path 54 from the nutrient source 14c, reagent source 14d, and gas source 14e, through the open inlet port 46, through the upper flow channel 38, through the outlet port 48 as spent culture media 26′, and to the waste destination 14i, thereby delivering nutrient, reagent, and gas to the environment surrounding the population of biological cells 2 residing in the bioreactor 12d, as well as extracting waste from the environment surrounding the population of biological cells 2 residing in the bioreactor 12d, and in particular, by allowing the nutrient, reagent, and gas within the fresh culture media 26 to diffuse through the porous membrane 36 from the upper flow channel 38 into the lower flow channel 40, and allowing the waste to diffuse through the porous membrane 36 from the lower flow channel 40 into the upper flow channel 38, to facilitate expansion of the population of biological cells 2.

As yet another example, the valve assembly 16 may be also operated to open the inlet port 50 associated with the lower flow channel 40 and the outlet port 48 associated with the upper flow channel 38, and to close the inlet port 46 associated with the upper flow channel 38 and the outlet port 52 associated with the lower flow channel 40, thereby defining the flow path 58 between the flow channels 38, 40 described above, while fluidly coupling the wash buffer source 14f (shown in FIG. 1) to the open inlet port 50 and fluidly coupling the waste destination 14i (shown in FIG. 1) to the open outlet port 48. As a result, under the fluid pressure applied by the pump assembly 18 (shown in FIG. 1), fresh wash buffer 28 flows along the flow path 58 from the biological cell source 14a and/or microbead source 14b, through the open inlet port 50, and into the lower flow channel 40; is then perfused through the porous membrane 36 from the lower flow channel 40 into the upper flow channel 38 (due to the pressure buildup in the lower flow channel 40 caused by the closed outlet port 52 that creates a pressure gradient between the flow channels 38, 40) as used wash buffer 28′ with undesirable material (e.g., unbound molecules, debris, dead cells, or other unwanted molecules), while the porous membrane 36 captures (filters) the biological cells 2 and/or microbeads 4, thereby retaining the biological cells 2 and/or microbeads 4 within the lower flow channel 40; and then flowed from the upper flow channel 38, through the outlet port 48, and to the waste destination 14i. In the same manner that the perfusion of the seeding media 24 with the biological cells 2 and/or microbeads 4 through the porous membrane 36 minimizes loss of biological cells 2 and/or microbeads 4 from the bioreactor 12d, perfusion of the used wash buffer 28′ containing undesirable material through the porous membrane 36 minimizes loss of the biological cells 2 and/or microbeads 4 out of the bioreactor 12d.

As yet another example, the valve assembly 16 may also be operated to open the inlet port 50 and outlet port 52 associated with the lower flow channel 40, and to close the inlet port 46 and outlet port 48 associated with the upper flow channel 38, thereby defining the flow path 56 entirely through the lower flow channel 40, while fluidly coupling the media source 14g (shown in FIG. 1) to the open inlet port 50 and fluidly coupling the biological cell collection destination 14h (shown in FIG. 1) to the open outlet port 52. As a result, under the fluid pressure applied by the pump assembly 18 (shown in FIG. 1), harvesting media 30 containing no biological cells 2 and microbeads 4 flows along the flow path 56 from the media source 14g, through the open inlet port 50, and into the lower flow channel 40; suspending the expanded population of biological cells 2 and microbeads 4 within the harvesting media 30; and flowing the harvesting media 30′ with the expanded population of biological cells 2 and microbeads 4, through the outlet port 52, and to the biological cell collection destination 14h, thereby harvesting the expanded population of biological cells 2 from the lower flow channel 40.

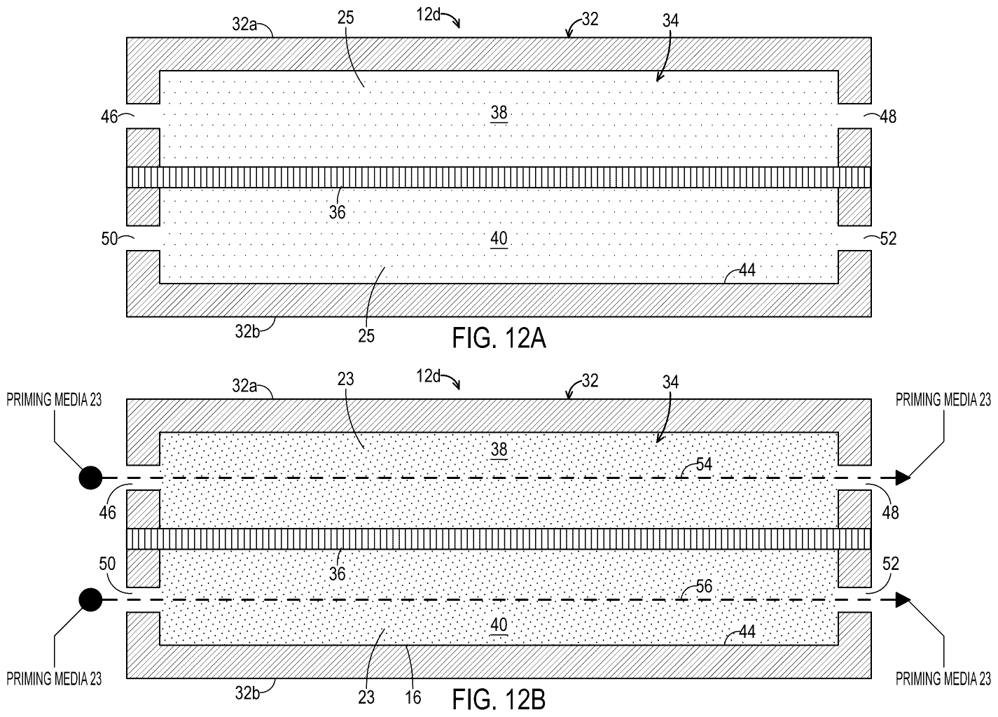

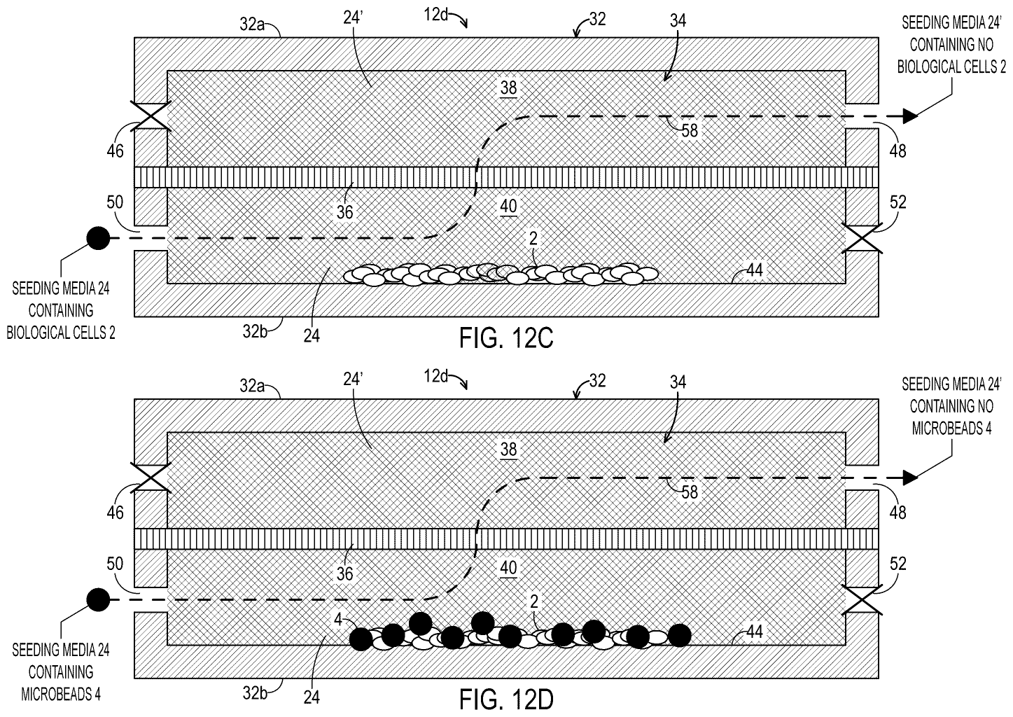

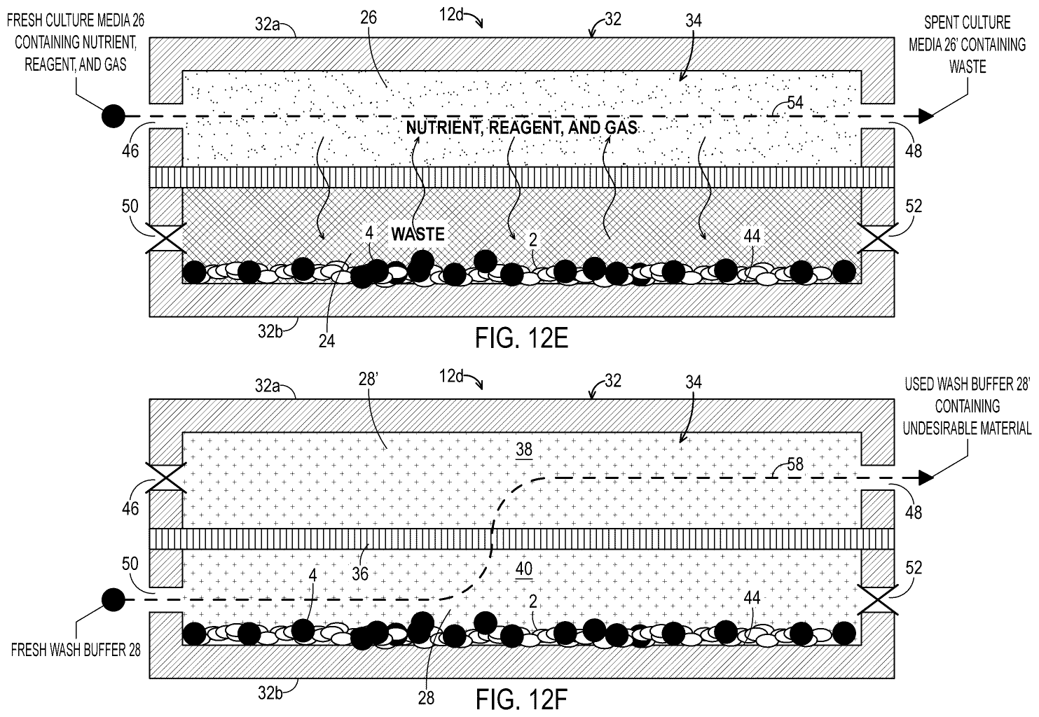

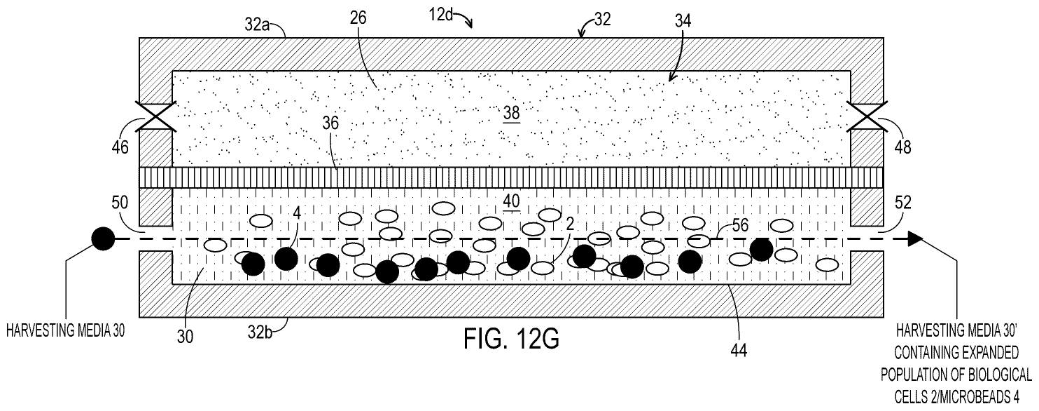

Having described the structure and function of the bioreactor 12d, one method 200 of operating the bioreactor 12d to expand and harvest a population of biological cells 2 (e.g., T lymphocytes (T-cells)) will now be described with reference to FIGS. 11 and 12A-12G. The method 150 is similar to the method 100 described above with respect to FIGS. 4 and 5A-5G, with the exception that the seeding media 24, culture media 26, washer buffer 28, and harvesting media 30 are flowed through opposite flow channels.

First, air 25 is flushed out of the flow channels 38, 40. Preferably, as much air as possible, but not necessarily all of the air 25, is flushed out of the flow channel 38, 40. In particular, the flow channels 38, 40 are primed with priming media 23 by flowing the priming media 23 through both the upper flow channel 38 and lower flow channel 40 until the air 25 (see FIG. 12A) is displaced (i.e., flushed out) from the flow channels 38, 40 (step 202) (see FIG. 12B). In the same manner described above with respect to step 102 of the method 100, the priming media 23 may be either concurrently or sequentially flowed through the upper flow channel 38 and lower flow channel 40.

Next, the lower flow channel 40 of the bioreactor 12d is seeded with the population of biological cells 2, such that the population of biological cells 2 rest on the cell culture surface 44 within the lower flow channel 40. In particular, the seeding media 24 with the biological cells 2 is flowed into the lower flow channel 40, and perfused through the porous membrane 36 from the lower flow channel 40 into the upper flow channel 38 while the biological cells 2 are retained within the lower flow channel 40, and then the seeding media 24′ without the biological cells 2 is then flowed out of the upper flow channel 38, thereby disposing a seed population of biological cells 2 on the cell culture surface 44 at the bottom of the lower flow channel 40 via the action of gravity (step 204) (see FIG. 12C).

As one example, while the inlet port 50 associated with the lower flow channel 40 and the outlet port 48 associated with the upper flow channel 38 are open, and the inlet port 46 associated with the upper flow channel 38 and the outlet port 52 associated with the lower flow channel 40 are closed, thereby defining only the flow path 58 between the flow channels 38, 40, the seeding media 24 with the biological cells 2 is flowed along the flow path 58 in through the inlet port 50 and into the lower flow channel 40, and perfused through the porous membrane 36 from the lower flow channel 40 to the upper flow channel 38 while the porous membrane 36 captures (filters) the biological cells 2, and then the seeding media 24′ without the biological cells 2 is further flowed along the flow path 58 through the upper flow channel 38 and out through the outlet port 48.

Optionally, microbeads 4 are disposed within the lower flow channel 40, such that the population of biological cells 2 adhere to the microbeads 4. In particular, the seeding media 24 with the microbeads 4 is flowed into the lower flow channel 40, perfused through the porous membrane 36 from the lower flow channel 40 into the upper flow channel 38 while the microbeads 4 are retained within the lower flow channel 40, and then the seeding media 24′ without the microbeads 4 is flowed out of the upper flow channel 38, such that microbeads 4 adhere to population of biological cells 2 (step 206) (see FIG. 12D).

As one example, while the inlet port 50 associated with the lower flow channel 40 and the outlet port 48 associated with the upper flow channel 38 are open, and the inlet port 46 associated with the upper flow channel 38 and the outlet port 52 associated with the lower flow channel 40 are closed, thereby defining only the flow path 58 between the flow channels 38, 40, the seeding media 24 with the microbeads 4 is flowed along the flow path 58 in through the inlet port 50 and into the lower flow channel 40, perfused through the porous membrane 36 from the lower flow channel 40 to the upper flow channel 38 while the porous membrane 36 captures (filters) the microbeads 4, and then the seeding media 24′ without the microbeads 4 is flowed further along the flow path 58 through the upper flow channel 38 and out through the outlet port 48.

While the microbeads 4 have been described as being delivered separately from the biological cells 2, into the lower flow channel 40, it should be appreciated that the microbeads 4 may be delivered with the biological cells 2, into the lower flow channel 40.

Next, nutrient, reagent, and gas are delivered to the population of biological cells 2, and waste is extracted from the population of biological cells 2, thereby facilitating the expansion of the population of biological cells 2 within the lower flow channel 40. In particular, the fresh culture media 26 is flowed (continually or intermittently) into the upper flow channel 38, such that nutrient, reagent, gas, and waste are exchanged across the porous membrane 36 between the population of biological cells 2 and culture media 26 (i.e., the nutrient, reagent, and gas are provided to, while the waste is extracted from, the population of biological cells 2, with the nutrient, reagent, and gas diffusing through the porous membrane 36 from the culture media 26 in the upper flow channel 38 to the seeding media 24 in the lower flow channel 40, and the waste diffusing through the porous membrane 36 from the seeding media 24 in the lower flow channel 40 to the culture media 26 in the upper flow channel 38), and then the spent culture media 26′ with the waste is flowed out of the upper flow channel 38, thereby facilitating expansion of the population of biological cells 2 (step 208) (see FIG. 12E). Significantly, diffusion of the nutrient, reagent, gas, and waste through the porous membrane 36 occurs while allowing the biological cells 2 and microbeads 4 to continue to stably rest on the cell culture surface 44.

As one example, while the inlet port 46 and outlet port 48 associated with the upper flow channel 38 are open, and the inlet port 50 and outlet port 52 associated with the lower flow channel 40 are closed, thereby defining only the flow path 54 entirely through the upper flow channel 38, the fresh culture media 26 containing the nutrient, reagent, and gas is flowed along the flow path 54 in through the inlet port 46 and into the upper flow channel 38, and then the spent culture media 26′ with the waste is flowed further along the flow path 54 through the upper flow channel 38 and out through the outlet port 48. For the same reasons discussed above with respect to the method 100 of operating the bioreactor 12a of FIGS. 2-3, it is preferred that culture media 26 be replaced within the upper flow channel 38 with fresh culture media 26 before the culture media 26 becomes completely spent; i.e., prior to the seeding media 24 and the culture media 26 achieving homogeneity.

Anytime during incubation, the population of biological cells 2 may be washed. In particular, much like the seeding of the lower flow channel 40 with the population of biological cells 2 with or without the microbeads 4, the fresh wash buffer 28 is flowed into the lower flow channel 40, and the used wash buffer 28′ containing undesirable material (e.g., unbound molecules, debris, dead cells, or other unwanted molecules) is perfused through the porous membrane 36 from the lower flow channel 40 into the upper flow channel 38 while the biological cells 2 and microbeads 4 are retained within the lower flow channel 40, and then flowed out of the upper flow channel 38, thereby washing the population of biological cells 2 and microbeads 4 (step 210) (see FIG. 12F).

As one example, while the inlet port 50 associated with the lower flow channel 40 and the outlet port 48 associated with the upper flow channel 38 are open, and the inlet port 46 associated with the upper flow channel 38 and the outlet port 52 associated with the lower flow channel 40 are closed, thereby defining only the flow path 58 between the flow channels 38, 40, the washer buffer 28 is flowed along the flow path 58 in through the inlet port 50 and into the lower flow channel 40, perfused through the porous membrane 36 from the lower flow channel 40 to the upper flow channel 38 while the porous membrane 36 captures (filters) the biological cells 2 and microbeads 4, and used washer buffer 30 with the undesirable material is flowed further along the flow path 58 through the upper flow channel 38 and out through the outlet port 48.

In an alternative method, the fresh wash buffer 28 may be flowed (continually or intermittently) into the upper flow channel 38, such that the active components of the fresh wash buffer 28 diffuse through the porous membrane 36 from the fresh wash buffer 28 in the upper flow channel 38 to the media in the lower flow channel 40. The media with the undesirable material may be then flowed out of the upper flow channel 38.

As one example, while the inlet port 46 and outlet port 48 associated with the upper flow channel 38 are open, and the inlet port 50 and outlet port 52 associated with the lower flow channel 40 are closed, thereby defining only the flow path 54 entirely through the upper flow channel 38, the fresh wash buffer 28 may be flowed along the flow path 54 in through the inlet port 46 and into the lower flow channel 40, and then the used wash buffer 28′ without the active components (or significantly less active components) is flowed further along the flow path 54 through the upper flow channel 38 and out through the outlet port 48. Then, while the inlet port 50 and outlet port 52 associated with the lower flow channel 40 are open, and the inlet port 46 and outlet port 48 associated with the upper flow channel 38 are closed, thereby defining only the flow path 56 entirely through the lower flow channel 40, media (e.g., PBS) may be flowed along the flow path 56 in through the inlet port 50, through the lower flow channel 40, and out through the outlet port 52, thereby removing the media with the undesirable material from the lower flow channel 40. It should be appreciated that in this alternative method, the media must be flowed through the lower flow channel 40 at a relatively low rate, such that the biological cells 2 and/or microbeads 4 are not suspended and lost out of the lower flow channel 40.

Notably, the washing step, can be performed without mechanically agitating the lower flow channel 40, since the population of biological cells 2 do not have to be adhered by structural functionalization of the cell culture surface 44 to withstand high tangential flow rates through the lower flow channel 40. In an optional method, the lower flow channel 40 may be slightly agitated (e.g., in the manner discussed above with respect to step 110 of method 100) to suspend the population of biological cells 2 and microbeads 4 within the seeding media 24 immediately prior to the washing step, so that the biological cells 2 do not hinder the perfusion of the wash buffer 19 through the porous membrane 36. This washing step can be performed several times.

The culture media 26 can then be again flowed through the upper flow channel 38 for exchange of the nutrient, gas, reagent, and waste between the population of biological cells 2 and the culture media 26 (step 208).

When ready to be harvested, the expanded population of biological cells 2 are harvested from the lower flow channel 40.

In particular, harvesting media 30 is flowed into the lower flow channel 40, such that the expanded population of biological cells 2 and microbeads 4 are suspended in the harvesting media 30, and then the harvesting media 30′ with the expanded population of biological cells 2 and microbeads 4 is flowed out of the lower flow channel 40, thereby extracting the expanded population of biological cells 2 from the bioreactor 12d (step 212) (see FIG. 12G).

As one example, while the inlet port 50 and outlet port 52 associated with the lower flow channel 40 are open, and the inlet port 46 and outlet port 48 associated with the upper flow channel 38 are closed, thereby defining only the flow path 56 entirely through the lower flow channel 40, the harvesting media 30 is flowed along the flow path 56 in through the inlet port 50 and into the lower flow channel 40, and the harvesting media 30′ with the expanded population of biological cells 2 and microbeads 4 is flowed further along the flow path 56 through the lower flow channel 40 and out through the outlet port 52.

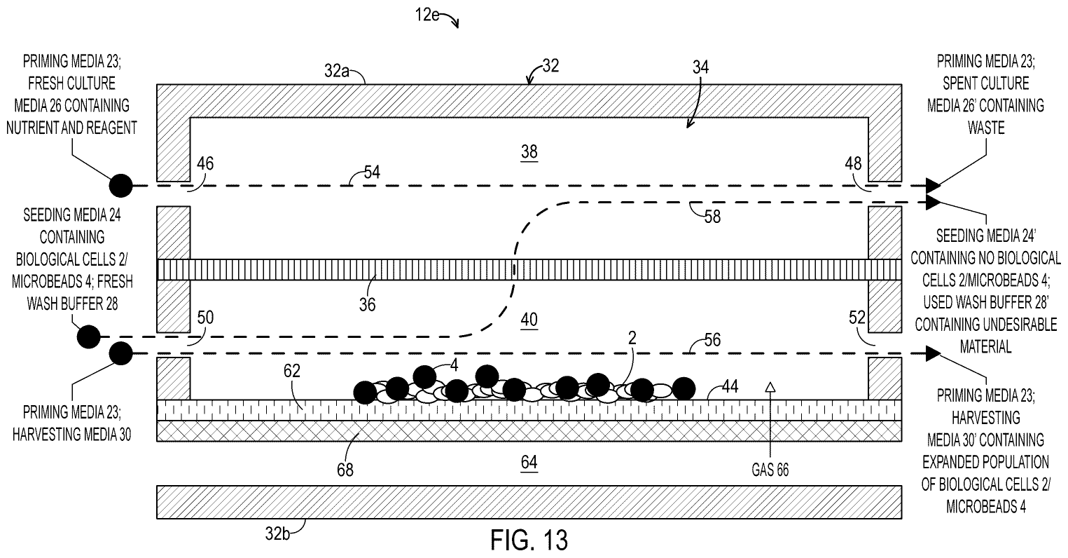

Referring now to FIG. 13, yet another embodiment of a bioreactor 12e for use in the bioreactor system 10 of FIG. 1 will now described. The bioreactor 12e is similar to the bioreactor 12d of FIG. 10, with the exception that the bioreactor 12e additionally comprises another membrane 62 affixed within the housing 32 at the bottom of the lower flow channel 40 spaced apart from and opposite the porous membrane 36, and on which the cell culture surface 44 is located. An ambient environment 64 is located below the other membrane 62, such that gas 66 (in this case, air) may be delivered through the other membrane 62 to the population of biological cells 2 via diffusion, while the nutrient and reagent from the culture media 26 and waste from the population of biological cells 2 diffuses across the porous membrane 36 between the flow channels 38, 40, as discussed above. In an alternative embodiment, a gas chamber (not shown) with an inlet port and an outlet port may be located below the other membrane 62, such that the gas 66 may be flowed within the gas chamber in a controlled manner.

To this end, the other membrane 62 is substantially permeable to gas, such that the air 66 may diffuse from the ambient environment 64 into the lower flow channel 40 for take-up by the population of biological cells 2, but substantially impermeable to liquid (e.g., the priming media 23, seeding media 24, culture media 26, washer buffer 28, or harvesting media 30) in the lower flow channel 40. The other membrane 62 is preferably a dense membrane (i.e., a membrane presenting no detectable pores at the limits of electron microscopy) that is composed of a low friction biocompatible material, e.g., a polymer, such as silicone, polytetrafluoroethylene (PTFE), polyether ether ketone (PEEK), etc., to prevent the population of biological cells 2 from adhering to the cell culture surface 44. Preferably, the dense membrane 62 provides a short diffusion path for the gas between the ambient environment 64 and the lower flow channel 40. For example, the dense membrane 62 may have a relatively small thickness, e.g., in the range of 50-250 microns. In one specific embodiment, the thickness of the dense membrane 56 is 100 microns. Notably, the use of a dedicated gas flow channel 58 separate from the culture media 26 improves gas delivery to the population of biological cells 2. In an alternative embodiment, the other membrane 62 may be a porous, but hydrophobic, membrane. The bubble point of the membrane 62 should be relatively high (preferably, several times higher than the maximum pressure differential between the flow channels 38, 40.

In a similar manner as the bioreactor 12d of FIG. 10, by locating the cell culture surface 44 on which the population of biological cells 2 rest away from the porous membrane 36, the population of biological cells 2 will not be disturbed by any shear force that would otherwise result from the lateral flexing of the porous membrane 36 in response to a significant pressure differential between the flow channels 38, 40. It is preferred that the dense membrane 62, and thus the cell culture surface 44, be spaced away from the porous membrane 36 a minimal distance (e.g., at least 1 mm), such that the population of biological cells 2 resting on the cell culture surface 44 do not come into contact with the porous membrane 36. Not only does sufficient spacing between the population of biological cells 2 and the porous membrane 36 reduce or minimize any disturbance of the population of biological cells 2 when the porous membrane 36 lateral flexes in response to a significant pressure differential between the flow channels 38, 40, clogging of the pores within the porous membrane 36 with biological cells 2 will be avoided. As such, diffusion of nutrient, reagent, and waste across the porous membrane 36 between the flow channels 38, 40 will be more efficient. Furthermore, to the extent that the porous membrane 36 is not completely impermeable to the biological cells 2, maintaining the population of the biological cells 2 on the cell culture surface 44 away from the porous membrane 36 will prevent any loss of biological cells 2 (e.g., via creeping or suction) through the porous membrane 36.

In a similar manner as the bioreactor 12b of FIG. 6, the bioreactor 12e additionally comprises a permeable support structure 68 configured for reducing a lateral flex of the dense membrane 62 in response to a pressure differential between the lower flow channel 40 and the ambient environment 64. Because, the lower flow channel 40 will generally have a higher pressure than the pressure of the ambient environment 64, the permeable support structure 68 is affixed to the lower housing portion 32b between the bottom surface of the dense membrane 62 and the ambient environment 64 (e.g., via heat staking, thermal or ultrasonic welding, laminating, or simply being pinched in an interference fit) to reduce or minimize downward lateral flexing of the dense membrane 62 into the ambient environment 64 in response to a pressure differential resulting from the flow of seeding media 24, culture media 26, washer buffer 28, and harvesting media 30 through the lower flow channel 40 (i.e., the fluid pressure in the lower flow channel 40 becomes higher than the fluid pressure of the ambient environment 64. Thus, the population of biological cells 2 will not be disturbed by any shear force that would otherwise result from the lateral flexing of the dense membrane 62 in response to a significant pressure differential between the lower flow channel 40 and the ambient environment 64. As a result, the expansion of the population of biological cells 2 is improved.

In the embodiment illustrated in FIG. 13, the permeable support structure 68 is highly porous so as to not unduly restrict the diffusion of gas through the dense membrane 62 between the lower flow channel 38 and the ambient environment 64. For example, the permeable support structure 68 may comprise a honeycomb or grid structure (e.g., composed stainless steel or a polymer, such as, e.g., polycarbonate, polystryrene, COC, COP, polypropylene, etc.). The permeable support structure 68 may have a suitable thickness, e.g., in the range of 0.1-2 mm, and a high porosity, e.g., in the range of 10-90%. Although the bioreactor 12e is described and illustrated as comprising a single porous structure 68, the bioreactor 12e may alternatively comprise two permeable support structures between which the dense membrane 62 is sandwiched.

The valve assembly 16 (illustrated in FIG. 1) may be operated in the same manner that it is operated with respect to the bioreactor 12d described above, with the exception that valve assembly 16 does not fluidly couple the gas source 14e to the open outlet port 46 associated with the upper flow channel 38. Rather, air 66 naturally diffuses through the dense membrane 62 from the ambient environment 64 into the lower flow channel 40.

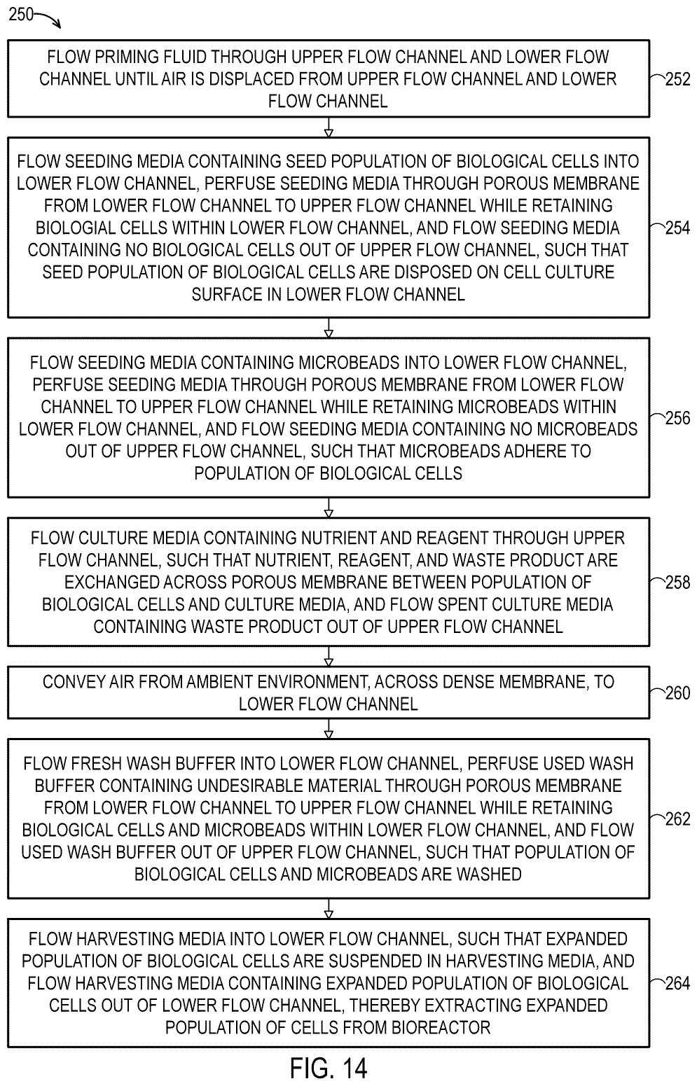

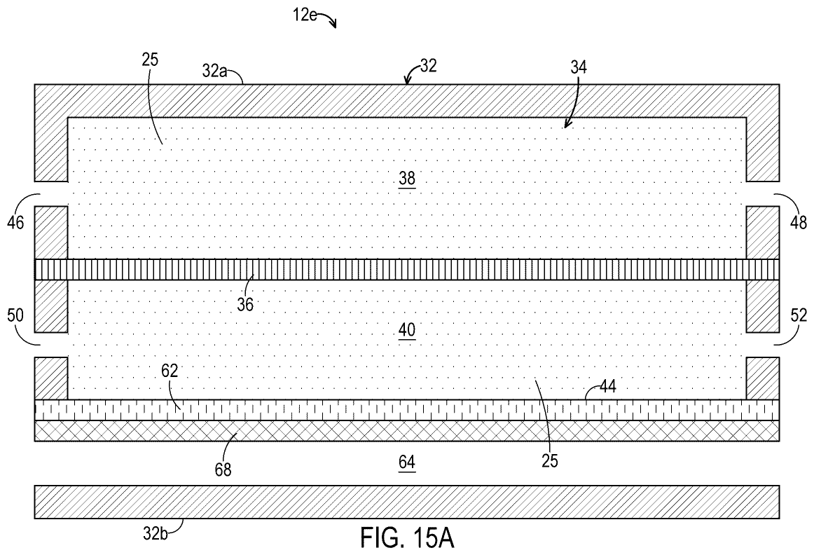

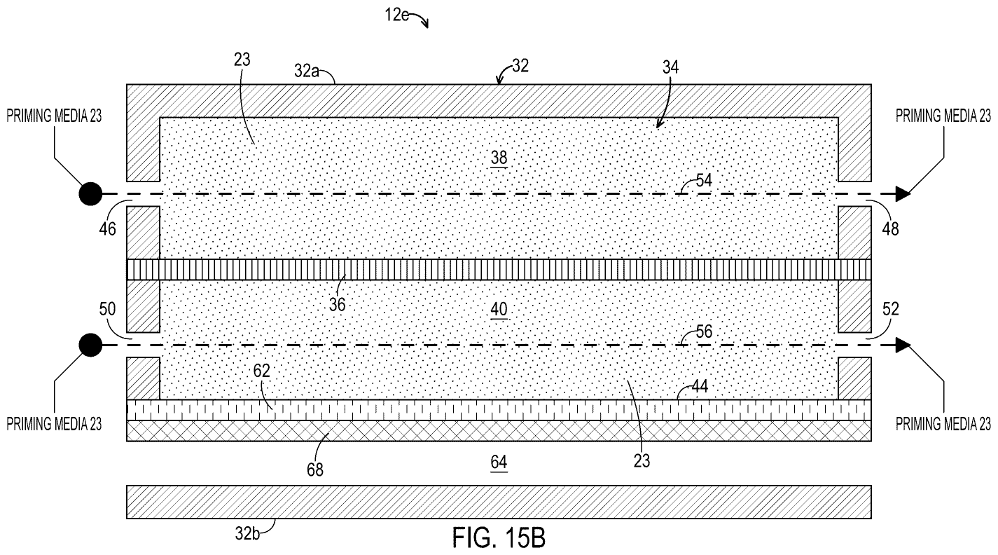

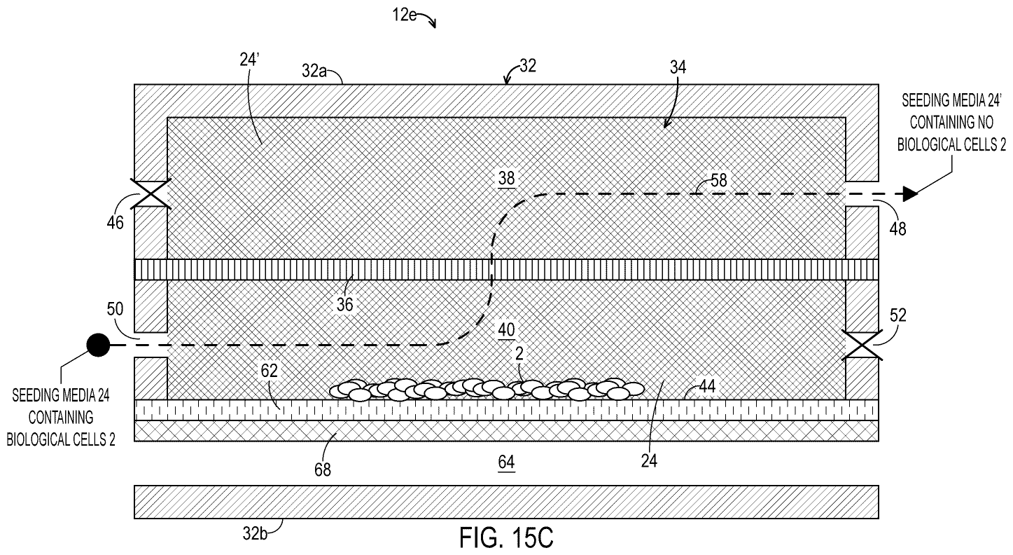

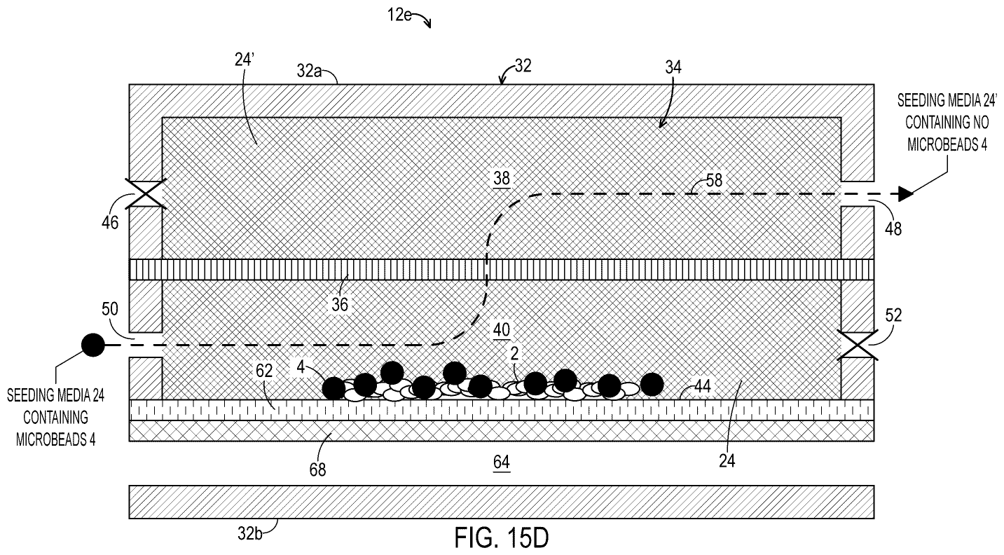

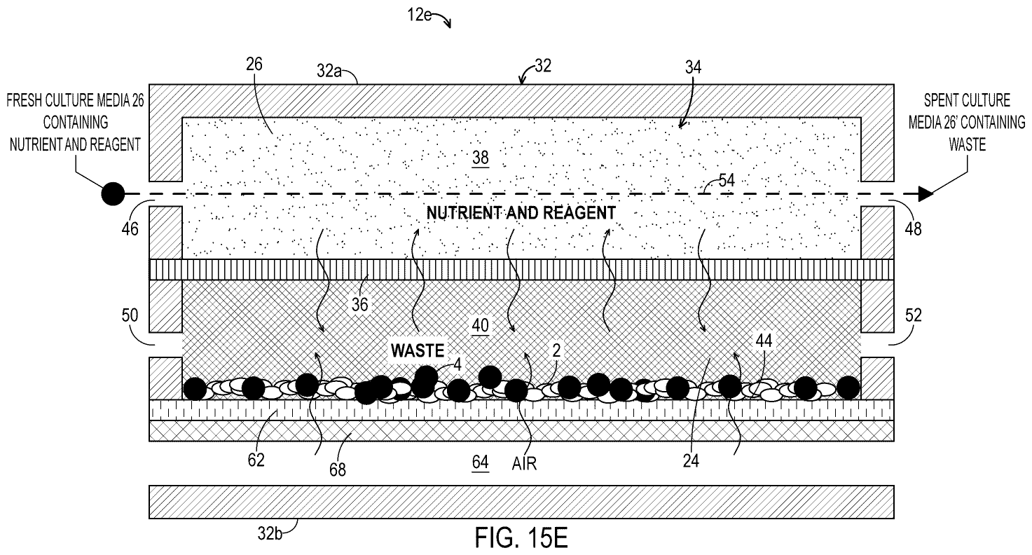

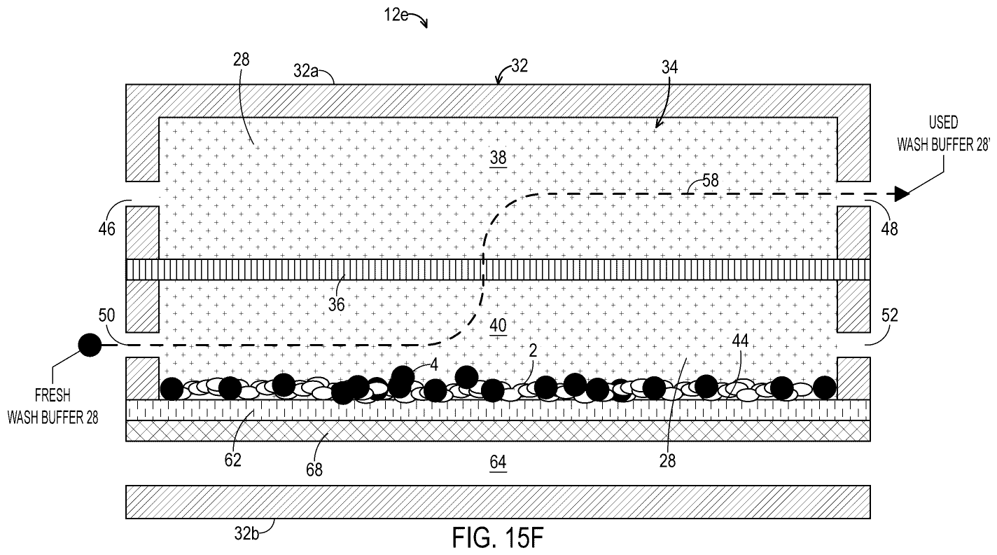

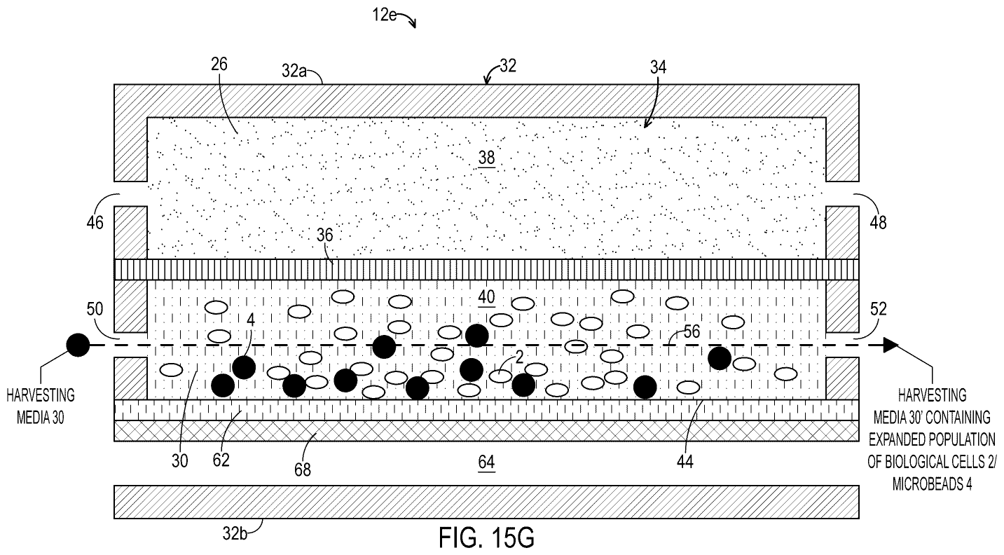

Having described the structure and function of the bioreactor 12e, one method 250 of operating the bioreactor 12e to expand and harvest a population of biological cells 2 (e.g., T lymphocytes (T-cells)) will now be described with reference to FIGS. 14 and 15A-15G. The method 250 is similar to the method 200 described above with respect to FIGS. 11 and 12A-12G, with the exception that gas is diffused into the lower flow channel 40 separately from the culture media 26.

First, air 25 is flushed out of the flow channels 38, 40. Preferably, as much air 25 as possible, but not necessarily all of the air 25, is flushed out of the flow channels 38, 40. In particular, the flow channels 38, 40 are primed with priming media 23 by flowing the priming media 23 through both the upper flow channel 38 and lower flow channel 40 until the air 25 (see FIG. 15A) is displaced (i.e., flushed out) from the flow channels 38, 40 (step 252) (see FIG. 15B). In the same manner described above with respect to step 102 of the method 100, the priming media 23 may be either concurrently or sequentially flowed through the upper flow channel 38 and lower flow channel 40.

Next, the lower flow channel 40 of the bioreactor 12e is seeded with the population of biological cells 2, such that the population of biological cells 2 rest on the cell culture surface 44 within the lower flow channel 40. In particular, the seeding media 24 with the biological cells 2 is flowed into the lower flow channel 40, and perfused through the porous membrane 36 from the lower flow channel 40 into the upper flow channel 38 while the biological cells 2 are retained within the lower flow channel 40, and then the seeding media 24′ without the biological cells 2 is then flowed out of the upper flow channel 38, thereby disposing a seed population of biological cells 2 on the cell culture surface 44 of the porous membrane 36 via the action of convection (step 204) (see FIG. 15C).

As one example, while the inlet port 50 associated with the lower flow channel 40 and the outlet port 48 associated with the upper flow channel 38 are open, and the inlet port 46 associated with the upper flow channel 38 and the outlet port 52 associated with the lower flow channel 40 are closed, thereby defining only the flow path 58 between the flow channels 38, 40, the seeding media 24 with the biological cells 2 is flowed along the flow path 58 in through the inlet port 50 and into the lower flow channel 40, and perfused through the porous membrane 36 from the lower flow channel 40 to the upper flow channel 38 while the porous membrane 36 captures (filters) the biological cells 2, and then the seeding media 24′ without the biological cells 2 is further flowed along the flow path 58 through the upper flow channel 38 and out through the outlet port 48.

Optionally, microbeads 4 are disposed within the lower flow channel 40, such that the population of biological cells 2 adhere to the microbeads 4. In particular, the seeding media 24 with the microbeads 4 is flowed into the lower flow channel 40, perfused through the porous membrane 36 from the lower flow channel 40 into the upper flow channel 38 while the microbeads 4 are retained within the lower flow channel 40, and then the seeding media 24′ without the microbeads 4 is flowed out of the upper flow channel 38, such that microbeads 4 adhere to population of biological cells 2 (step 206) (see FIG. 15D).

As one example, while the inlet port 50 associated with the lower flow channel 40 and the outlet port 48 associated with the upper flow channel 38 are open, and the inlet port 46 associated with the upper flow channel 38 and the outlet port 52 associated with the lower flow channel 40 are closed, thereby defining only the flow path 58 between the flow channels 38, 40, the seeding media 24 with the microbeads 4 is flowed along the flow path 58 in through the inlet port 50 and into the lower flow channel 40, perfused through the porous membrane 36 from the lower flow channel 40 to the upper flow channel 38 while the porous membrane 36 captures (filters) the microbeads 4, and then the seeding media 24′ without the microbeads 4 is flowed further along the flow path 58 through the upper flow channel 38 and out through the outlet port 48.

While the microbeads 4 have been described as being delivered separately from the biological cells 2, into the lower flow channel 40, it should be appreciated that the microbeads 4 may be delivered with the biological cells 2, into the lower flow channel 40.

Next, nutrient, reagent, and gas are delivered to the population of biological cells 2, and waste is extracted from the population of biological cells 2, thereby facilitating the expansion of the population of biological cells 2 within the lower flow channel 40.

In particular, the fresh culture media 26 is flowed (continually or intermittently) into the upper flow channel 38, such that nutrient, reagent, and waste are exchanged across the porous membrane 36 between the population of biological cells 2 and culture media 26 (i.e., the nutrient and reagent are provided to, while the waste is extracted from, the population of biological cells 2, with the nutrient and reagent diffusing through the porous membrane 36 from the culture media 26 in the upper flow channel 38 to the seeding media 24 in the lower flow channel 40, and the waste diffusing through the porous membrane 36 from the seeding media 24 in the lower flow channel 40 to the culture media 26 in the upper flow channel 38), and then the spent culture media 26′ with the waste is flowed out of the upper flow channel 38, thereby facilitating expansion of the population of biological cells 2 (step 258) (see FIG. 15E).

As one example, while the inlet port 46 and outlet port 48 associated with the upper flow channel 38 are open, and the inlet port 50 and outlet port 52 associated with the lower flow channel 40 are closed, thereby defining only the flow path 54 entirely through the upper flow channel 38, the fresh culture media 26 containing the nutrient and reagent is flowed along the flow path 54 in through the inlet port 46 and into the upper flow channel 38, and then the spent culture media 26′ with the waste is flowed further along the flow path 54 through the upper flow channel 38 and out through the outlet port 48. For the same reasons discussed above with respect to the method 100 of operating the bioreactor 12a of FIGS. 2-3, it is preferred that culture media 26 be replaced within the upper flow channel 38 with fresh culture media 26 before the culture media 26 becomes completely spent; i.e., prior to the seeding media 24 and the culture media 26 achieving homogeneity.

Simultaneously with the diffusion of the nutrient, reagent, and waste across the porous membrane 36 between the flow channels 38, 40, air 66 is diffused from the ambient environment 64, through the dense membrane 62, and into the lower flow channel 40, while allowing liquid media to be retained within the lower flow channel 40 (step 260) (see FIG. 15E). Significantly, diffusion of the nutrient, reagent, gas, and waste through the porous membrane 36 occurs while allowing the biological cells 2 and microbeads 4 to continue to stably rest on the cell culture surface 44.

Anytime during incubation, the population of biological cells 2 may be washed. In particular, much like the seeding of the lower flow channel 40 with the population of biological cells 2 with or without the microbeads 4, the fresh wash buffer 28 is flowed into the lower flow channel 40, and used wash buffer 28′ containing undesirable material (e.g., unbound molecules, debris, dead cells, or other unwanted molecules) is perfused through the porous membrane 36 from the lower flow channel 40 into the upper flow channel 38 while the biological cells 2 and microbeads 4 are retained within the lower flow channel 40, and then flowed out of the upper flow channel 38, thereby washing the population of biological cells 2 and microbeads 4 (step 262) (see FIG. 15F).

For example, while the inlet port 50 associated with the lower flow channel 40 and the outlet port 48 associated with the upper flow channel 38 are open, and the inlet port 46 associated with the upper flow channel 38 and the outlet port 52 associated with the lower flow channel 40 are closed, thereby defining only the flow path 58 between the flow channels 38, 40, the washer buffer 28 is flowed along the flow path 58 in through the inlet port 50 and into the lower flow channel 40, perfused through the porous membrane 36 from the lower flow channel 40 to the upper flow channel 38 while the porous membrane 36 captures (filters) the biological cells 2 and microbeads 4, and used washer buffer 30 with the undesirable material is flowed further along the flow path 58 through the upper flow channel 38 and out through the outlet port 48.

In an alternative method, fresh wash buffer 28 may be flowed (continually or intermittently) into the upper flow channel 38, such that the active components of the fresh wash buffer 28 diffuse through the porous membrane 36 from the fresh wash buffer 28 in the upper flow channel 38 to the media in the lower flow channel 40. The media with the undesirable material may be then flowed out of the upper flow channel 38.

As one example, while the inlet port 46 and outlet port 48 associated with the upper flow channel 38 are open, and the inlet port 50 and outlet port 52 associated with the lower flow channel 40 are closed, thereby defining only the flow path 54 entirely through the upper flow channel 38, the fresh wash buffer 28 may be flowed along the flow path 54 in through the inlet port 46 and into the lower flow channel 40, and then the used wash buffer 28′ containing undesirable material (e.g., unbound molecules, debris, dead cells, or other unwanted molecules) is flowed further along the flow path 54 through the upper flow channel 38 and out through the outlet port 48. Then, while the inlet port 50 and outlet port 52 associated with the lower flow channel 40 are open, and the inlet port 46 and outlet port 48 associated with the upper flow channel 38 are closed, thereby defining only the flow path 56 entirely through the lower flow channel 40, media (e.g., PBS) may be flowed along the flow path 56 in through the inlet port 50, through the lower flow channel 40, and out through the outlet port 52, thereby removing the media with the undesirable material from the lower flow channel 40. It should be appreciated that in this alternative method, the media must be flowed through the lower flow channel 40 at a relatively low rate, such that the biological cells 2 and/or microbeads 4 are not suspended and lost out of the lower flow channel 40.

Notably, the washing step, can be performed without mechanically agitating the lower flow channel 40, since the population of biological cells 2 do not have to be adhered by structural functionalization of the cell culture surface 44 to withstand high tangential flow rates through the lower flow channel 40. In an optional method, the lower flow channel 40 may be slightly agitated (e.g., in the manner discussed above with respect to step 110 of method 100) to suspend the population of biological cells 2 and microbeads 4 within the seeding media 24 immediately prior to the washing step, so that the biological cells 2 do not hinder the perfusion of the wash buffer 19 through the porous membrane 36. This washing step can be performed several times.

The culture media 26 can then be again flowed through the upper flow channel 38 for exchange of the nutrient, reagent, and waste between the population of biological cells 2 and the culture media 26 (step 258).

When ready to be harvested, the expanded population of biological cells 2 are harvested from the lower flow channel 40.

In particular, harvesting media 30 is flowed into the lower flow channel 40, such that the expanded population of biological cells 2 and microbeads 4 are suspended in the harvesting media 30, and then the harvesting media 30′ with the expanded population of biological cells 2 and microbeads 4 is flowed out of the lower flow channel 40, thereby extracting the expanded population of biological cells 2 from the bioreactor 12d (step 264) (see FIG. 15G).

For example, while the inlet port 50 and outlet port 52 associated with the lower flow channel 40 are open, and the inlet port 46 and outlet port 48 associated with the upper flow channel 38 are closed, thereby defining only the flow path 56 entirely through the lower flow channel 40, the harvesting media 30 is flowed along the flow path 56 in through the inlet port 50 and into the lower flow channel 40, and the harvesting media 30′ with the expanded population of biological cells 2 and microbeads 4 is flowed further along the flow path 56 through the lower flow channel and out through the outlet port 52.

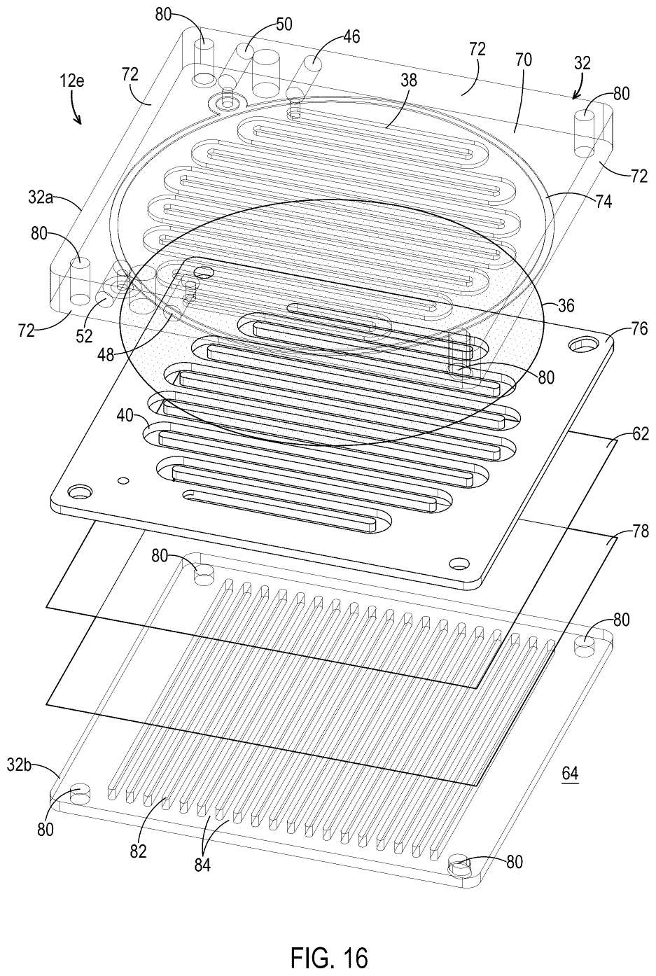

Referring to FIG. 16, one physical implementation of a bioreactor 12 (corresponding to the bioreactor 12e illustrated in FIG. 13) will now be described. In this implementation, the upper housing portion 32a of the housing 32 of the bioreactor 12′ takes the form of an open box-shaped container having an upper wall 70 and four sidewalls 72, while the lower housing portion 32b takes the form of a rigid plate that is affixed to the open end of the upper housing portion 32a to affix the components of the bioreactor 12′ within the housing 32. It should be appreciated that, although the housing portions 32a, 32b are square-shaped, the housing portions 32a, 32b may take the form of any shape, including rectangular, circular, oval, octagonal, etc.

The bioreactor 12′ comprises a recess 74 formed within the upper wall 70 of the upper housing portion 32a using a suitable process, e.g., injection molding or machining. In the illustrated embodiment, the recess 74 is circular, although in alternative embodiments, the recess 74 may take the form of any suitable shape, including a square, rectangular, oval, octagonal, etc. The upper flow channel 38 is formed within the recess 74 using a suitable process, e.g., injection molding or machining. In the illustrated embodiment, the upper flow channel 38 is serpentine in nature, although in alternative embodiments, the upper flow channel 38 may have any suitable shape, including a spiral shape. In this embodiment, the upper flow channel has a volume of 4 mL.

The bioreactor 12′ further comprises a rigid plate 76 disposed within the upper housing portion 32a below the recess 74. In the illustrated embodiment, the rigid plate 76 has a shape and size that matches the top wall 70 of the upper housing portion 32a, such that the periphery of the rigid plate 76 extends outside of the periphery of the recess 74. The lower flow channel 40 is formed entirely through the rigid plate 76 using a suitable process, e.g., injection molding or machining, such that the lower flow channel 40 is disposed below the upper flow channel 38 when the rigid plate 76 is affixed within the upper housing portion 32a. In the illustrated embodiment, the lower flow channel 40 is serpentine in nature, although in alternative embodiments, the lower flow channel 40 may have any suitable shape, including a spiral shape. It should be appreciated that although the flow channels 38, 40 have the same shape and size, they may have different shapes and sizes from each other. In this embodiment, the lower flow channel has a volume of 4 mL.

The porous membrane 36 is affixed within the recess 74, such that the porous membrane 36 is disposed between the upper flow channel 38 and the lower flow channel 40. The porous membrane 36 has the same size and shape as the recess 74, such that the entirety of the upper flow channel 38 is covered by the porous membrane 36. Thus, the flow channels 38, 40 are completely separated from each other by the porous membrane 36. The dense membrane 62 is affixed within the upper housing portion 32a below the rigid plate 76, such that the lower flow channel 40 is sandwiched between the porous membrane 36 and the dense membrane 62. In the illustrated embodiment, the dense membrane 62 has a shape and size that matches the top wall 70 of the upper housing portion 32a (and the rigid plate 76), such that the entireties of the opposite sides of the upper flow channel 38 are covered by the porous membrane 36 and the dense membrane 62. The permeable support structure 78 is sandwiched between the dense membrane 62 and the lower housing portion 32b. In the illustrated embodiment, the dense membrane 62 has a shape and size that matches the dense membrane 62, such that the entire surface of the dense membrane 62 is supported by the permeable support structure 78.

The bottom housing portion 32b takes the form of a base plate configured for being affixed to the top housing portion 32a, thereby stably integrating the porous membrane 36, rigid plate 76, dense membrane 62, and permeable support structure 78 together. For example, the housing portions 32a, 32b may have holes 80 through which fasteners (not shown) may be installed to affix the housing portions 32a, 32b together. The bottom housing portion 32b comprises an array of parallel rectilinear spacers 82 between which an array of parallel rectilinear channels 82 are formed. The rectilinear spacers 82 are configured for contacting the bottom surface of the permeable support structure 78 when the bottom housing portion 32b is affixed to the top housing portion 32a, while the rectilinear channels 84 are exposed to the ambient environment 64.

The inlet port 46 is formed in the upper wall 70 of the upper housing portion 32a, and is fluidly coupled to the beginning of the upper flow channel 38 inside the periphery of the recess 74 in which the upper flow channel 38 is formed, whereas the outlet port 48 is formed in the upper wall 70 of the housing portion 32a opposite to the inlet port 46, and is fluidly coupled to the end of the upper flow channel 38 inside the periphery of the recess 74 in which the upper flow channel 38 is formed. Similarly, the inlet port 50 is formed through the upper wall 70 of the upper housing portion 32a, and is fluidly coupled to the beginning of the lower flow channel 40, whereas the outlet port 52 is formed through the upper wall 70 of the housing portion 32a opposite to the inlet port 50, and is fluidly coupled to the end of the lower flow channel 40. The ports 46-52 may be formed in or through the upper wall 70 of the upper housing portion 32a using any suitable process, e.g., injection molding or machining.

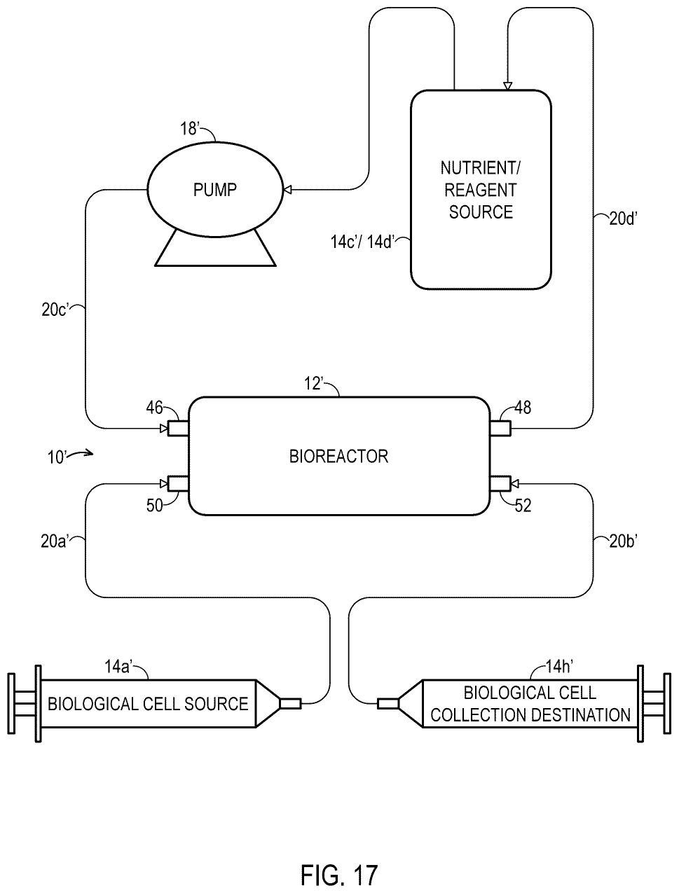

Referring now to FIG. 17, an experimental bioreactor system 10′ was built and tested to determine its capability of expanding a population of biological cells in comparison to a static bioreactor (in this case, a flask). The bioreactor system 10′ comprises the bioreactor 12′ (similar to the bioreactor 12d illustrated in FIG. 10), a biological cell source 14a′ (in the form of a syringe), a biological cell collection destination 14h′ (in the form of a syringe), a nutrient/reagent source 14c′/14d′, a first conduit 20a′ fluidly coupling the biological cell source 14a′ to the inlet port 50 of the bioreactor 12′, a second conduit 20b′ fluidly coupling the biological cell collection destination 14h′ to the outlet port 52 of the bioreactor 12′, a third conduit 20c′ fluidly coupling the nutrient/reagent source 14c′/14d′ to the inlet port 46 of the bioreactor 12′, a fourth conduit 20d′ fluidly coupling the nutrient/reagent source 14c′/14d′ to the outlet port 48 of the bioreactor 12′, and a peristaltic pump 18′ fluidly coupled in the third conduit 20c′ between the nutrient/reagent source 14c′/14d′ to the inlet port 46 of the bioreactor 12′.

Thus, the syringe 14a′ may be operated to flow seeding media 24 containing a seed population of biological cells 2 into the lower flow channel 40 (shown in FIG. 13) of the bioreactor 12′, such that the population of biological cells 2 are disposed on the cell culture surface 44 within the lower flow channel 40, while the syringe 14b′ may be operated to flow harvesting media 30 containing an expanded population of biological cells 2 out of the lower flow channel 40 (shown in FIG. 13) of the bioreactor 12′ in the same manner described above with respect to steps 254 and 264 of the method 250 illustrated in FIG. 14. The peristaltic pump 18′ may be operated to flow cell culture media 26 through the upper flow channel 38, thereby exchanging nutrient, reagent, and waste between the biological cells 2 and the cell culture media 26 (shown in FIG. 13).

In one experimental case (Case A), the upper flow channel 38 was seeded with a population of 5 million biological cells 12 via operation of the syringe 14a′, after which culture media 26 was continuously flowed through the lower flow channel 40 (approximately 3.5-4.0 mL/min). In another experimental case (Case B), the upper flow channel 38 was seeded with a population of 5 million biological cells 12 via operation of the syringe 14a′, after which culture media 26 was intermittently flowed through the lower flow channel 40 (3-4 mL/min for 120 s, off for 180 s, repeating once every 300 s). In a Control Case, a static bioreactor (a flask) was seeded with a population of 5 million biological cells 12.

As shown below in Table A, the bioreactor 12′ of Case A and B is capable of expanding a population of biological cells on par with the expansion of biological cells by the Control Case, and even significantly outpaces the expansion of biological cells by the Control Case prior to the 10^{th} day. Unlike the static bioreactor in the Control Case, however, a bioreactor system 10′ that comprises the bioreactor 12′ as a base unit, may be scaled up by incrementally adding bioreactors 12′ to further expand a population of biological cells 2.

TABLE A

| | | Case A | | Case B | | Control Case | |
|---|---|---|---|---|---|---|---|
| | Days Since | Cells | Fold | Cells | Fold | Cells | Fold |
| Date | Start (Day) | (M) | Expansion | (M) | Expansion | (M) | Expansion |
| Jun. 13, 2022 | 0.0 | 5 | 1.0 | 5 | 1.0 | 5 | 1.0 |
| 5:00pm | | | | | | | |
| Jun. 16, 2022 | 2.9 | 29.5 | 5.9 | 20.8 | 4.2 | 15.3 | 3.1 |
| 2:00pm | | | | | | | |
| Jun. 21, 2022 | 7.8 | 234 | 46.8 | 246.0 | 49.2 | 165.3 | 33.1 |
| 12:00pm | | | | | | | |
| Jun. 23, 2022 | 9.9 | 230 | 46.0 | 248.0 | 49.6 | 242.2 | 48.4 |
| 2:00pm | | | | | | | |

Although particular embodiments have been shown and described herein, it will be understood by those skilled in the art that they are not intended to limit the disclosed inventions, and it will be obvious to those skilled in the art that various changes, permutations, and modifications may be made (e.g., the dimensions of various parts, combinations of parts) without departing from the scope of the disclosed inventions, which is to be defined only by the following claims and their equivalents. The specification and drawings are, accordingly, to be regarded in an illustrative rather than restrictive sense. The various embodiments shown and described herein are intended to cover alternatives, modifications, and equivalents of the disclosed inventions, which may be included within the scope of the appended claims.

### NUMBERED EMBODIMENTS OF THE INVENTION

1. A bioreactor for expanding a population of biological cells, comprising a housing having an interior cavity; a first membrane affixed within the housing, the first membrane dividing the interior cavity into a chamber and a first flow channel; a cell culture surface contained within the chamber on which the population of biological cells may be disposed; a first inlet port fluidly coupled to the first flow channel, such that first media can be flowed into the first flow channel; and a first outlet port fluidly coupled to the first flow channel, such that the first media can be flowed out of the first flow channel; wherein the first membrane is substantially permeable to one or more of nutrient, reagent, and gas contained within the first media and waste produced by the population of biological cells, while being substantially impermeable to the population of biological cells.
2. The bioreactor of embodiment 1, wherein the first media contains the one or more of the nutrient, reagent, and gas, and the first membrane is substantially permeable to the one or more of the nutrient, reagent, and gas.
3. The bioreactor of embodiment 1 or 2, wherein the first membrane is substantially permeable to the waste produced by the population of biological cells.
4. The bioreactor of any one of embodiments 1-3, wherein the first membrane is configured for allowing diffusion of the one or more of nutrient, reagent, and gas from the first flow channel into the chamber, and diffusion of the waste from the chamber into the first flow channel, thereby facilitating expansion of the population of biological cells within the chamber.
5. The bioreactor of any one of embodiments 1-4, wherein the first membrane has a thickness in the range of 10-100 microns.
6. The bioreactor of any one of embodiments 1-5, wherein the chamber is a static chamber.
7. The bioreactor of any one of embodiments 1-6, wherein the cell culture surface is formed on the first membrane.
8. The bioreactor of any one of embodiments 1-6, wherein the cell culture surface is spaced from the first membrane.
9. The bioreactor of embodiment 8, wherein the cell culture surface is spaced at least 1 mm from the first membrane.
10. The bioreactor of embodiment 8 or 9, wherein the cell culture surface is formed on a wall of the housing opposite the first membrane.
11. The bioreactor of any one of embodiments 1-5 and 7-10, wherein the chamber is a second flow channel, the bioreactor further comprising a second inlet port fluidly coupled to the second flow channel, such that second media containing biological cells can be flowed into the second flow channel; and a second outlet port fluidly coupled to the second flow channel, such that third media containing an expanded population of biological cells may be flowed out of the second flow channel.
12. The bioreactor of embodiment 11, wherein each of the first flow channel and the second flow channel is planar.
13. The bioreactor of embodiment 12, wherein each of the first flow channel and the second flow channel is patterned.
14. The bioreactor of embodiment 13, wherein each of the first flow channel and the second flow channel has a serpentine pattern.
15. The bioreactor of any one of embodiments 11-14, wherein each of the first flow channel and the second flow channel has a volume in the range of 2-100 ml.
16. The bioreactor of any one of embodiments 11-14, wherein the first membrane is a porous membrane configured for allowing perfusion of the second media from the second flow channel into the first flow channel, while filtering the population of biological cells from the second media, such that the population of biological cells are retained within the second flow channel.
17. The bioreactor of embodiment 16, wherein the porous membrane is composed of a polycarbonate.
18. The bioreactor of embodiment 16 or 17, wherein the porous membrane comprises pores having diameters in the range of 0.05-0.4 microns.
19. The bioreactor of any one of embodiments 16-18, wherein the porous membrane has a porosity in the range of 1%-20%.
20. The bioreactor of any one of embodiments 11-19, further comprising at least one permeable support structure configured for reducing a lateral flex of the porous membrane in response to a pressure differential between the first flow channel and the second flow channel.
21. The bioreactor of embodiment 20, wherein each of the at least one permeable support structure comprises a woven mesh.
22. The bioreactor of embodiment 20 or 21, wherein each of the at least one permeable support structure has a porosity in the range of 10-90%.
23. The bioreactor of any of embodiments 1-6, further comprising a second membrane affixed within the housing, wherein the chamber is disposed between the first membrane and the second membrane, and wherein the second membrane is substantially permeable to gas, while being substantially impermeable to the population of biological cells and the second media.
24. The bioreactor of embodiment 23, wherein the second membrane is configured for allowing diffusion of the gas from a space exterior to the second flow channel into the chamber, thereby facilitating expansion of the population of biological cells within the second flow channel.
25. The bioreactor of embodiment 24, wherein the exterior space is an ambient environment.
26. The bioreactor of embodiment 24 or 25, further comprising a permeable support structure configured for reducing a lateral flex of the second membrane in response to a pressure differential between the chamber and the space exterior to the second flow channel.
27. The bioreactor of embodiment 26, wherein the permeable support structure comprises a honeycomb structure.
28. The bioreactor of embodiment 26 or 27, wherein the permeable support structure is composed of one of polycarbonate, polystyrene, cyclic olefin copolymer (COC), cyclic olefin polymer (COP), polypropylene, and stainless steel.
29. The bioreactor of any one of embodiments 26-28, wherein the permeable support structure has a thickness in the range of 0.1-2 mm.
30. The bioreactor of any one of embodiments 26-29, wherein the permeable support structure has a porosity in the range of 10-90%.
31. The bioreactor of any one of embodiments 23-30, wherein the cell culture surface is formed on the second membrane.
32. The bioreactor of any one of embodiments 23-31, wherein the second membrane is a dense membrane.
33. The bioreactor of any one of embodiments 23-32, wherein the second membrane is composed of silicone.
34. The bioreactor of any one of embodiments 23-33, wherein the second membrane has a thickness in the range of 50-250 microns.
35. A bioreactor system, comprising the bioreactor of any one of embodiments 11-34; and a pump assembly fluidly coupled to the first inlet port and the second inlet port.
36. The bioreactor system of embodiment 35, wherein the pump assembly comprises a peristaltic pump.
37. The bioreactor system of embodiment 35 or 36, further comprising a valve assembly configured for alternately allowing and preventing the flow of fluid through each of the first inlet port, the first outlet port, the second inlet port, and the second outlet port.
38. A method of operating the bioreactor of any one of embodiments 1-34, comprising seeding the chamber with the population of biological cells, such that the population of biological cells are disposed on the cell culture surface; flowing the first media through the first inlet port and into the first flow channel; conveying the one or more of nutrient, reagent, and gas from the first flow channel, through the first membrane, and into the chamber, thereby facilitating the expansion of the population of biological cells within the chamber; flowing the first media out of the first flow channel through the first outlet port; and harvesting the expanded population of biological cells from the chamber.
39. The method of embodiment 38, further comprising conveying the waste from the chamber, through the first membrane, and into the first flow channel.
40. The method of embodiment 39, wherein conveying the one or more of nutrient, reagent, and gas from the first flow channel, through the first membrane, and into the chamber comprises diffusing the one or more of nutrient, reagent, and gas from the first flow channel, through the first membrane, and into the chamber, and conveying the waste from the chamber, through the first membrane, and into the first flow channel comprises diffusing the waste from the chamber, through the first membrane, and into the first flow channel.
41. The method of any one of embodiments 38-40, wherein flowing the first media through the first inlet port and into the first flow channel, and flowing the first media out of the first flow channel through the first outlet port comprises pumping the first media.
42. The method of any one of embodiments 38-41, wherein the biological cells are T lymphocytes (T-cells).
43. A method of operating the bioreactor of any one of embodiments 11-22, comprising flowing the second media containing the population of biological cells through the second inlet port and into the second flow channel, such that the population of biological cells are disposed on the cell culture surface; flowing the first media through the first inlet port and into the first flow channel; conveying the one or more of nutrient, reagent, and gas from the first flow channel, through the first membrane, and into the second flow channel, thereby facilitating the expansion of the population of biological cells within the second flow channel; flowing the first media out of the first flow channel through the first outlet port; flowing third media through the second inlet port and into the second flow channel, such that the expanded population of biological cells is suspended in the third media; and flowing the third media with the suspended expanded population of biological cells out of the second flow channel and through the second outlet port.
44. The method of embodiment 43, further comprising perfusing the second media from the second flow channel, through the first membrane, and into the first flow channel, while substantially retaining the population of biological cells within the second flow channel; and flowing the perfused second media out of the first flow channel through the first outlet port.
45. The method of embodiment 43 or 44, further comprising conveying the waste from the second flow channel, through the first membrane, and into the first flow channel.
46. The method of embodiment 45, wherein conveying the one or more of nutrient, reagent, and gas from the first flow channel, through the first membrane, and into the second flow channel comprises diffusing the one or more of nutrient, reagent, and gas from the first flow channel, through the first membrane, and into the second flow channel, and conveying the waste from the second flow channel, through the first membrane, and into the first flow channel comprises diffusing the waste from the second flow channel, through the first membrane, and into the first flow channel.
47. The method of any one of embodiments 43-46, wherein the expanded population of biological cells is suspended within the third media without mechanically agitating the second flow channel.
48. The method of any one of embodiments 43-47, further comprising flowing a wash buffer through the second inlet port and into the second flow channel, thereby washing the population of biological cells; perfusing the wash buffer from the second flow channel, through the first membrane, and into the first flow channel, while substantially retaining the population of biological cells within the second flow channel; and flowing the perfused wash buffer out of the first flow channel through the first outlet port.
49. The method of any one of embodiments 43-48, wherein flowing the first media through the first inlet port and into the first flow channel, and flowing the first media out of the first flow channel through the first outlet port comprises pumping the first media; wherein flowing the second media through the second inlet port and into the second flow channel comprises pumping the second media; and wherein flowing the third media through the second inlet port and into the second flow channel, and flowing the third media out of the second flow channel and through the second outlet port comprises pumping the third media.
50. The method of any one of embodiments 43-49, wherein the biological cells are T lymphocytes (T-cells).
51. A method of operating the bioreactor of any one of embodiments 23-34, comprising seeding the chamber with the population of biological cells, such that the population of biological cells are disposed on the cell culture surface; flowing the first media through the first inlet port and into the first flow channel; conveying one or more of nutrient and reagent contained within the first media from the first flow channel, through the first membrane, and into the chamber, and conveying the gas from a space exterior of the chamber, through the second membrane, and into the chamber, thereby facilitating the expansion of the population of biological cells within the chamber; flowing the first media out of the first flow channel through the first outlet port; and harvesting the expanded population of biological cells from the chamber.
52. The method of embodiment 51, further comprising conveying the waste from the chamber, through the first membrane, and into the first flow channel.
53. The method of embodiment 52, wherein conveying the one or more of nutrient, reagent, and gas from the first flow channel, through the first membrane, and into the chamber comprises diffusing the one or more of nutrient, reagent, and gas from the first flow channel, through the first membrane, and into the chamber, and conveying the waste from the chamber, through the first membrane, and into the first flow channel comprises diffusing the waste from the chamber, through the first membrane, and into the first flow channel.
54. The method of any one of embodiments 51-53, wherein flowing the first media through the first inlet port and into the first flow channel, and flowing the first media out of the first flow channel through the first outlet port comprises pumping the first media.
55. The method of any one of embodiments 51-54, wherein the biological cells are T lymphocytes (T-cells).
56. A method of expanding a population of biological cells, comprising seeding a chamber with the population of biological cells, such that the population of biological cells rest on a cell culture surface within the chamber; flowing first media containing one or more of nutrient, reagent, and gas through a first flow channel; diffusing the one or more of nutrient, reagent, and gas from the first flow channel into the chamber, thereby expanding the population of biological cells within the chamber; and harvesting the expanded population of biological cells from the chamber.
57. The method of embodiment 56, further comprising diffusing waste produced by the population of biological cells from the chamber into the first flow channel.
58. The method of embodiment 56 or 57, wherein the chamber is a second flow channel, wherein the second flow channel is seeded by flowing second media containing the biological cells into the second flow channel, such that the population of biological cells are disposed on the cell culture surface, and wherein the expanded population of biological cells is harvested from the incubation flow channel by flowing third media into the second flow channel, such that the expanded population of biological cells is suspended in the third media, and flowing the third media with the suspended expanded population of biological cells out of the second flow channel.
59. The method of embodiment 58, further comprising perfusing the second media from the second flow channel into the first flow channel, while substantially retaining the population of biological cells within the second flow channel; and flowing the perfused second media out of the first flow channel.
60. The method of any one of embodiments 56-59 wherein the expanded population of biological cells is suspended within the third media without mechanically agitating the second flow channel.
61. The method of any one of embodiments 56-60, further comprising flowing a wash buffer within the second flow channel, thereby washing the population of biological cells; perfusing the wash buffer from the second flow channel into the first flow channel, while substantially retaining the population of biological cells within the second flow channel; and flowing the perfused wash buffer out of the first flow channel.
62. The method of any one of embodiments 56-61, wherein the first media is continuously flowed through the first flow channel.
63. The method of any one of embodiments 56-61, wherein the first media is intermittently flowed through the first flow channel.
64. The method of any one of embodiments 56-63, wherein flowing the first media through the first flow channel comprises pumping the first media.
65. The method of any one of embodiments 56-64, wherein the biological cells are T lymphocytes (T-cells).
66. A bioreactor for expanding a population of biological cells, comprising a housing having an interior cavity; a porous membrane affixed within the housing, the porous membrane dividing the interior cavity into a first flow channel and a second flow channel; a cell culture surface contained within the second flow channel on which the population of biological cells may be disposed; a first inlet port fluidly coupled to the second flow channel, such that first media containing biological cells can be flowed into the second flow channel, wherein the porous membrane is configured for allowing perfusion of the first media from the second flow channel into the first flow channel, while filtering the population of biological cells from the first media, such that the population of biological cells are retained within the second flow channel; and a first outlet port fluidly coupled to the first flow channel, such that the first media can be flowed out of the first flow channel.
67. The bioreactor of embodiment 66, wherein the porous membrane is substantially permeable to one or more of nutrient, reagent, and gas contained within second media disposed within the first flow channel.
68. The bioreactor of embodiment 66 or 67, wherein the porous membrane is substantially permeable to the waste produced by the waste produced by the population of biological cells.
69. The bioreactor of embodiment 68, wherein the porous membrane is configured for allowing diffusion of the one or more of nutrient, reagent, and gas from the first flow channel into the second flow channel, and diffusion of the waste from the second flow channel into the first flow channel, thereby facilitating expansion of the population of biological cells within the chamber.
70. The bioreactor of any one of embodiments 66-69, further comprising a second inlet port fluidly coupled to the first flow channel, such that the second media can be flowed into the first flow channel, and flowed out of the first outlet port.
71. The bioreactor of any one of embodiments 66-70, wherein the porous membrane has a thickness in the range of 10-100 microns.
72. The bioreactor of any one of embodiments 66-71, wherein the cell culture surface is formed on the porous membrane.
73. The bioreactor of any one of embodiments 66-71, wherein the cell culture surface is spaced from the first membrane.
74. The bioreactor of embodiment 73, wherein the cell culture surface is spaced at least 1 mm from the first membrane.
75. The bioreactor of embodiment 73 or 74, wherein the cell culture surface is formed on a wall of the housing opposite the first membrane.
76. The bioreactor of any one of embodiments 66-75, further comprising a second outlet port fluidly coupled to the second flow channel, such that second media containing an expanded population of biological cells may be flowed out of the second flow channel.
77. The bioreactor of any one of embodiments 66-75, wherein each of the first flow channel and the second flow channel is planar.
78. The bioreactor of embodiment 77, wherein each of the first flow channel and the second flow channel is patterned.
79. The bioreactor of embodiment 78, wherein each of the first flow channel and the second flow channel has a serpentine pattern.
80. The bioreactor of any one of embodiments 66-79, wherein each of the first flow channel and the second flow channel has a volume in the range of 2-100 ml.
81. The bioreactor of any one of embodiments 66-80, wherein the porous membrane is composed of a polycarbonate.
82. The bioreactor of any one of embodiments 66-81, wherein the porous membrane comprises pores having diameters in the range of 0.05-0.4 microns.
83. The bioreactor of any one of embodiments 66-82, wherein the porous membrane has a porosity in the range of 1%-20%.
84. The bioreactor of any one of embodiments 66-83, further comprising at least one permeable support structure configured for reducing a lateral flex of the porous membrane in response to a pressure differential between the first flow channel and the second flow channel.
85. The bioreactor of embodiment 84, wherein each of the at least one permeable support structure comprises a woven mesh.
86. The bioreactor of embodiment 84 or 85, wherein each of the at least one permeable support structure has a porosity in the range of 10-90%.
87. The bioreactor of any one of embodiments 66-86, further comprising a dense membrane affixed within the housing, wherein the second flow channel is disposed between the porous membrane and the dense membrane, and wherein the dense membrane is substantially permeable to gas, while being substantially impermeable to the population of biological cells and liquid media.
88. The bioreactor of embodiment 87, wherein the dense membrane is configured for allowing diffusion of the gas from a space exterior to the second flow channel into the second flow channel, thereby facilitating expansion of the population of biological cells within the second flow channel.
89. The bioreactor of embodiment 88, wherein the exterior space is an ambient environment.
90. The bioreactor of any one of embodiments 87-89, further comprising a permeable support structure configured for reducing a lateral flex of the dense membrane in response to a pressure differential between the second flow channel and the space exterior to the second flow channel.
91. The bioreactor of embodiment 90, wherein the permeable support structure comprises a honeycomb structure.
92. The bioreactor of embodiment 90 or 91, wherein the permeable support structure is composed of one of polycarbonate, polystryrene, cyclic olefin copolymer (COC), cyclic olefin polymer (COP), polypropylene, and stainless steel.
93. The bioreactor of any one of embodiments 90-92, wherein the permeable support structure has a thickness in the range of 0.1-2 mm.
94. The bioreactor of any one of embodiments 90-93, wherein the permeable support structure has a porosity in the range of 10-90%.
95. The bioreactor of any one of embodiments 87-94, wherein the cell culture surface is formed on the dense membrane.
96. The bioreactor of any one of embodiments 87-95, wherein the dense membrane is composed of silicone.
97. The bioreactor of any one of embodiments 87-96, wherein the dense membrane has a thickness in the range of 50-250 microns.
98. A bioreactor system, comprising the bioreactor of any one of embodiments 66-97; and a pump assembly fluidly coupled to the first inlet port.
98. The bioreactor system of embodiment 97, wherein pump assembly comprises a peristaltic pump.

99. A method of operating the bioreactor of any one of embodiments 66-98, comprising flowing the first media containing the population of biological cells through the first inlet port and into the second flow channel; perfusing the first media from the second flow channel, through the porous membrane, and into the first flow channel, while substantially retaining the population of biological cells within the second flow channel, such that the population of biological cells are disposed on the cell culture surface; and flowing the perfused first media out of the first flow channel through the first outlet port.
100. The method of embodiment 99, further comprising flowing a wash buffer through the first inlet port and into the second flow channel, thereby washing the population of biological cells; perfusing the wash buffer from the second flow channel, through the porous membrane, and into the first flow channel, while substantially retaining the population of biological cells within the second flow channel; and flowing the perfused wash buffer out of the first flow channel through the first outlet port.
101. The method of embodiment 99 or 100, wherein the flowing the first media through the first inlet port and into the second flow channel, perfusing the first media from the second flow channel, through the porous membrane, and flowing the perfused first media out of the first flow channel through the first outlet port comprises pumping the first media.
102. The method of any one of embodiments 99-101, wherein the biological cells are T lymphocytes (T-cells).
103. A method of operating the bioreactor of embodiment 70, flowing the first media containing the population of biological cells through the first inlet port and into the second flow channel; perfusing the first media from the second flow channel, through the porous membrane, while substantially retaining the population of biological cells within the second flow channel, such that the population of biological cells are disposed on the cell culture surface; flowing the perfused first media out of the first flow channel through the first outlet port; flowing the second media through the second inlet port and into the first flow channel; conveying the one or more of nutrient, reagent, and gas from the first flow channel, through the first membrane, and into the second flow channel, thereby facilitating the expansion of the population of biological cells within the second flow channel; and flowing the second media out of the first flow channel through the first outlet port.
104. The method of embodiment 103, further comprising conveying waste produced by the population of biological cells from the second flow channel, through the porous membrane, and into the first flow channel.
105. The method of embodiment 104, wherein conveying the one or more of nutrient, reagent, and gas from the first flow channel, through the porous membrane, and into the second flow channel comprises diffusing the one or more of nutrient, reagent, and gas from the first flow channel, through the porous membrane, and into the second flow channel, and conveying the waste from the second flow channel, through the porous membrane, and into the first flow channel comprises diffusing the waste from the second flow channel, through the porous membrane, and into the first flow channel.
106. The method of any one of embodiments 103-105, further comprising flowing a wash buffer through the first inlet port and into the second flow channel, thereby washing the population of biological cells; perfusing the wash buffer from the second flow channel, through the porous membrane, and into the first flow channel, while substantially retaining the population of biological cells within the second flow channel; and flowing the perfused wash buffer out of the first flow channel through the first outlet port.
107. The method of any one of embodiments 103-106, wherein the flowing the first media through the first inlet port and into the second flow channel, perfusing the first media from the second flow channel, through the porous membrane, and flowing the perfused first media out of the first flow channel through the first outlet port comprises pumping the first media; and wherein flowing the second media through the second inlet port and into the first flow channel, and flowing the second media out of the first flow channel through the first outlet port comprises pumping the second media.
108. The method of any one of embodiments 103-107, further comprising harvesting the expanded population of biological cells from the second flow channel.
109. The method of any one of embodiments 103-108, wherein the biological cells are T lymphocytes (T-cells).
110. A method of operating the bioreactor of embodiment 76, flowing the first media containing the population of biological cells through the first inlet port and into the second flow channel; perfusing the first media from the second flow channel, through the porous membrane, and into the first flow channel, while substantially retaining the population of biological cells within the second flow channel, such that the population of biological cells are disposed on the cell culture surface; flowing the perfused first media out of the first flow channel through the first outlet port; flowing the second media through the first inlet port and into the second flow channel, such that the expanded population of biological cells is suspended in the second media; and flowing the second media with the suspended expanded population of biological cells out of the second flow channel and through the second outlet port.
109. The method of embodiment 108, wherein the expanded population of biological cells is suspended within the second media without mechanically agitating the second flow channel.
110. The method of embodiment 108 or 109, further comprising flowing a wash buffer through the first inlet port and into the second flow channel, thereby washing the population of biological cells; perfusing the wash buffer from the second flow channel, through the porous membrane, and into the first flow channel, while substantially retaining the population of biological cells within the second flow channel; and flowing the perfused wash buffer out of the first flow channel through the first outlet port.
111. The method of any one of embodiments 108-110, wherein flowing the first media through the first inlet port and into the second flow channel, perfusing the first media from the second flow channel, through the porous membrane, and into the first flow channel, and flowing the perfused first media out of the first flow channel through the first outlet port comprises pumping the first media; and wherein flowing the second media through the first inlet port and into the second flow channel, and flowing the second media with the suspended expanded population of biological cells out of the second flow channel comprises pumping the second media.
112. The method of any one of embodiments 108-111, wherein the biological cells are T lymphocytes (T-cells).
113. A method of seeding a bioreactor with biological cells, comprising flowing first media containing biological cells into a first flow channel; perfusing the first media from first flow channel into the second flow channel, while substantially retaining the population of biological cells within the second flow channel, such that the population of biological cells are disposed on a cell culture surface within the first flow channel; flowing the perfused first media out of the second flow channel.
114. The method of embodiment 113, further comprising flowing a wash buffer within the first flow channel, thereby washing the biological cells; perfusing the wash buffer from the first flow channel into the second flow channel, while substantially retaining the biological cells within the first flow channel; and flowing the perfused wash buffer out of the second flow channel.
115. The method of embodiment 113 or 114, further comprising flowing second media containing one or more of nutrient, reagent, and gas through the second flow channel; and diffusing the one or more of nutrient, reagent, and gas from the second flow channel into the first flow channel, thereby expanding the population of biological cells within the first flow channel.
116. The method of embodiment 115, further comprising flowing third media into the first flow channel, such that the expanded population of biological cells is suspended in the third media, and flowing the third media with the suspended expanded population of biological cells out of the first flow channel.
117. The method of embodiment 116, wherein the expanded population of biological cells is suspended within the third media without mechanically agitating the second flow channel.
118. The method of any one of embodiments 115-117, wherein the second media is continuously flowed through the second flow channel.
119. The method of any one of embodiments 115-117, wherein the second media is intermittently flowed through the second flow channel.
120. The method of any one of embodiments 113-119, wherein flowing the first media into a first flow channel, perfusing the first media from first flow channel into the second flow channel, and flowing the perfused first media out of the second flow channel comprises pumping the first media.
121. The method of any one of embodiments 113-120, wherein the biological cells are T lymphocytes (T-cells).
122. A bioreactor, comprising a housing; a first membrane within the housing and at least partially defining (i) a first flow channel on a first side of the first porous membrane for flow of first media containing one or more of nutrient and reagent and (ii) a chamber on a second side of the first membrane for a population of biological cells to be disposed within; and a second membrane within the housing and at least partially defining an exterior space on a first side of the second membrane for introduction of a gas to the population of biological cells, the second membrane having a second side within the chamber upon which the population of biological cells is to be disposed.
123. The bioreactor of embodiment 122, wherein the first membrane is configured for allowing diffusion of the one or more of nutrient and reagent from the first flow channel into the chamber, and diffusion of waste from the chamber into the first flow channel, and the second membrane is configured for allowing diffusion of the gas from a space exterior to the second flow channel into the second flow channel, thereby facilitating expansion of the population of biological cells within the second flow channel.
124. The bioreactor of embodiment 122 or 123, wherein the first membrane has a thickness in the range of 10-100 microns, and the second membrane has a thickness in the range of 50-250 microns.
125. The bioreactor of any one of embodiments 122-124, wherein the chamber is a static chamber.
126. The bioreactor of any one of embodiments 122-124, where the chamber is a second flow channel for flow of second media containing the population of biological cells.
127. The bioreactor of embodiment 126, wherein each of the first flow channel and the second flow channel is planar.
128. The bioreactor of embodiment 127, wherein each of the first flow channel and the second flow channel is patterned.
129. The bioreactor of embodiment 128, wherein each of the first flow channel and the second flow channel has a serpentine pattern.
130. The bioreactor of any one of embodiments 126-129, wherein each of the first flow channel and the second flow channel has a volume in the range of 2-100 ml.
131. The bioreactor of any one of embodiments 126-130, wherein the first membrane is a porous membrane configured for allowing perfusion of the second media from the second flow channel into the first flow channel, while filtering the population of biological cells from the second media, such that the population of biological cells are retained within the second flow channel.
132. The bioreactor of embodiment 131, wherein the porous membrane is composed of a polycarbonate, and the second membrane is composed of silicone.
133. The bioreactor of embodiment 131 or 132, wherein the porous membrane comprises pores having diameters in the range of 0.05-0.4 microns.
134. The bioreactor of any one of embodiments 131-133, wherein the porous membrane has a porosity in the range of 1%-20%.
135. The bioreactor of any one of embodiments 122-134, wherein the second membrane is a dense membrane.
136. The bioreactor of any one of embodiments 122-135, wherein the second membrane at least partially defines an ambient environment on first side of the second membrane for introduction of the gas to the population of biological cells.
137. The bioreactor of any one of embodiments 122-136, further comprising a permeable support structure configured for reducing a lateral flex of the second membrane in response to a pressure differential between the second flow channel and the space exterior to the second flow channel.
138. The bioreactor of embodiment 137, wherein the permeable support structure comprises a honeycomb structure.
139. The bioreactor of embodiment 137 or 148, wherein the permeable support structure is composed of one of polycarbonate, polystryrene, cyclic olefin copolymer (COC), cyclic olefin polymer (COP), polypropylene, and stainless steel.
140. The bioreactor of any one of embodiments 137-139, wherein the permeable support structure has a thickness in the range of 0.1-2 mm.
141. The bioreactor of any one of embodiments 137-140, wherein the permeable support structure has a porosity in the range of 10-90%.
142. A bioreactor system, comprising the bioreactor of any one of embodiments 122-141; and a pumping assembly configured to provide the first media to the first flow channel.
143. The bioreactor system of embodiment 142, further comprising a controller configured to operate the pumping assembly.

1. A bioreactor for expanding a population of biological cells, comprising:
a housing having an interior cavity;
a first membrane affixed within the housing, the first membrane dividing the interior cavity into a chamber and a first flow channel;
a cell culture surface contained within the chamber on which the population of biological cells may be disposed;
a first inlet port fluidly coupled to the first flow channel, such that first media can be flowed into the first flow channel; and
a first outlet port fluidly coupled to the first flow channel, such that the first media can be flowed out of the first flow channel;
wherein the first membrane is substantially permeable to one or more of nutrient, reagent, and gas contained within the first media and waste produced by the population of biological cells, while being substantially impermeable to the population of biological cells.

2-3. (canceled)

4. The bioreactor of claim 1, wherein the first membrane is configured for allowing diffusion of the one or more of nutrient, reagent, and gas from the first flow channel into the chamber, and diffusion of the waste from the chamber into the first flow channel, thereby facilitating expansion of the population of biological cells within the chamber.

5. (canceled)

6. The bioreactor of claim 1, wherein the chamber is a static chamber.

7. The bioreactor of claim 1, wherein the cell culture surface is formed on the first membrane.

8. The bioreactor of claim 1, wherein the cell culture surface is spaced from the first membrane.

9. (canceled)

10. The bioreactor of claim 8, wherein the cell culture surface is formed on a wall of the housing opposite the first membrane.

11. The bioreactor of claim 1, wherein the chamber is a second flow channel, the bioreactor further comprising:
a second inlet port fluidly coupled to the second flow channel, such that second media containing biological cells can be flowed into the second flow channel; and
a second outlet port fluidly coupled to the second flow channel, such that third media containing an expanded population of biological cells may be flowed out of the second flow channel.

12. The bioreactor of claim 11, wherein each of the first flow channel and the second flow channel is planar.

13-14. (canceled)

15. The bioreactor of claim 11, wherein each of the first flow channel and the second flow channel has a volume in the range of 2-100 ml.

16. The bioreactor of claim 11, wherein the first membrane is a porous membrane configured for allowing perfusion of the second media from the second flow channel into the first flow channel, while filtering the population of biological cells from the second media, such that the population of biological cells are retained within the second flow channel.

17-18. (canceled)

19. The bioreactor of claim 16, wherein the porous membrane has a porosity in the range of 1%-20%.

20. The bioreactor of claim 11, further comprising at least one permeable support structure configured for reducing a lateral flex of the porous membrane in response to a pressure differential between the first flow channel and the second flow channel.

21-22. (canceled)

23. The bioreactor of claim 1, further comprising a second membrane affixed within the housing, wherein the chamber is disposed between the first membrane and the second membrane, and wherein the second membrane is substantially permeable to gas, while being substantially impermeable to the population of biological cells and the second media.

24. The bioreactor of claim 23, wherein the second membrane is configured for allowing diffusion of the gas from a space exterior to the second flow channel into the chamber, thereby facilitating expansion of the population of biological cells within the second flow channel.

25. The bioreactor of claim 24, wherein the exterior space is an ambient environment.

26. The bioreactor of claim 24, further comprising a permeable support structure configured for reducing a lateral flex of the second membrane in response to a pressure differential between the chamber and the space exterior to the second flow channel.

27-30. (canceled)

31. The bioreactor of claim 23, wherein the cell culture surface is formed on the second membrane.

32. The bioreactor of claim 21, wherein the second membrane is a dense membrane.

33-34. (canceled)

35. A bioreactor system, comprising:
the bioreactor of claim 11; and
a pump assembly fluidly coupled to the first inlet port and the second inlet port.

36. (canceled)

37. The bioreactor system of claim 35, further comprising a valve assembly configured for alternately allowing and preventing the flow of fluid through each of the first inlet port, the first outlet port, the second inlet port, and the second outlet port.
